(19) United States
(12) Patent Application Publication
(10) Pub. No.: US 2024/0293387 A1
(43) Pub. Date: Sep. 5, 2024

(54) Methods of Treating Fabry Disease in Pediatric Patients

(71) Applicant: Amicus Therapeutics, Inc., Philadelphia, PA (US)

(72) Inventor: Franklin Johnson, Bridgewater, NJ (US)

(21) Appl. No.: 18/578,483

(22) PCT Filed: Jul. 12, 2022
(86) PCT No.: PCT/US2022/073626

§ 371 Date: Jan. 11, 2024

Related U.S. Application Data
(60) Provisional application No. 63/220,816, filed on Jul. 12, 2021.

(51) Int. Cl. A61K 31/445; A61P 3/00

(52) U.S. Cl. CPC A61K 31/445; A61P 3/00

(57) ABSTRACT

Provided are methods for the treatment of Fabry disease in adolescent patient.

### TECHNICAL FIELD

Principles and embodiments of the present invention relate generally to the treatment of Fabry disease.

### BACKGROUND

Many human diseases result from mutations that cause changes in the amino acid sequence of a protein which reduce its stability and may prevent it from folding properly. Proteins generally fold in a specific region of the cell known as the endoplasmic reticulum, or ER. The cell has quality control mechanisms that ensure that proteins are folded into their correct three-dimensional shape before they can move from the ER to the appropriate destination in the cell, a process generally referred to as protein trafficking. Misfolded proteins are often eliminated by the quality control mechanisms after initially being retained in the ER. In certain instances, misfolded proteins can accumulate in the ER before being eliminated. The retention of misfolded proteins in the ER interrupts their proper trafficking, and the resulting reduced biological activity can lead to impaired cellular function and ultimately to disease. In addition, the accumulation of misfolded proteins in the ER may lead to various types of stress on cells, which may also contribute to cellular dysfunction and disease.

Such mutations can lead to lysosomal storage disorders (LSDs), which are characterized by deficiencies of lysosomal enzymes due to mutations in the genes encoding the lysosomal enzymes. The resultant disease causes the pathologic accumulation of substrates of those enzymes, which include lipids, carbohydrates, and polysaccharides. Although there are many different mutant genotypes associated with each LSD, many of the mutations are missense mutations which can lead to the production of a less stable enzyme. These less stable enzymes are sometimes prematurely degraded by the ER-associated degradation pathway. This results in the enzyme deficiency in the lysosome, and the pathologic accumulation of substrate. Such mutant enzymes are sometimes referred to in the pertinent art as “folding mutants” or “conformational mutants.”

Fabry disease, an LSD, is a progressive, X-linked inborn error of glycosphingolipid metabolism caused by a deficiency in the lysosomal enzyme α-galactosidase A (α-Gal A) as a result of mutations in the α-Gal A gene (GLA). Despite being an X-linked disorder, females can express varying degrees of clinical manifestations.

Fabry disease is classified by clinical manifestations into three groups: a classic form with generalized vasculopathy, an atypical variant form with clinical manifestations limited to cardiac tissue, and later-onset disease, which includes female carriers with mild to severe forms of the disease. The clinical manifestations include angiokeratoma (small, raised reddish-purple blemishes on the skin), acroparesthesias (burning in hands and feet), hypohidrosis (decreased ability to sweat), and characteristic corneal and lenticular opacities (The Metabolic and Molecular Bases of Inherited Disease, 8th Edition 2001, Scriver et al., ed., pp. 3733-3774, McGraw-Hill, New York).

Fabry is a rare disease with incidence estimated between 1 in 40,000 males to 1 in 117,000 in the general population. Moreover, there are variants of later onset phenotype of Fabry disease that can be under-diagnosed, as they do not present with classical signs and symptoms. This, and newborn screening for Fabry disease, suggests that the actual incidence of Fabry disease can be higher than currently estimated.

Untreated, life expectancy in Fabry patients is reduced and death usually occurs in the fourth or fifth decade because of vascular disease affecting the kidneys, heart and/or central nervous system. The enzyme deficiency leads to intracellular accumulation of the substrate, globotriaosylceramide (GL-3) in the vascular endothelium and visceral tissues throughout the body. The heart may also become enlarged and the kidneys may become progressively involved. Gradual deterioration of renal function and the development of azotemia, due to glycosphingolipid deposition, usually occur in the third to fifth decades of life, but can occur as early as in the second decade. Renal lesions are found in both hemizygous (male) and heterozygous (female) patients. The affected male's life expectancy is reduced, and death usually occurs in the fourth or fifth decade as a result of vascular disease of the heart, brain, and/or kidneys. Other symptoms include fever and gastrointestinal difficulties, particularly after eating.

Cardiac disease as a result of Fabry disease occurs in most males and many females. Early cardiac findings include left ventricular enlargement, valvular involvement and conduction abnormalities. Mitral insufficiency is the most frequent valvular lesion typically present in childhood or adolescence. Cerebrovascular manifestations result primarily from multifocal small-vessel involvement and can include thromboses, transient ischemic attacks, basilar artery ischemia and aneurysm, seizures, hemiplegia, hemianesthesia, aphasia, labyrinthine disorders, or cerebral hemorrhages. Average age of onset of cerebrovascular manifestations is 33.8 years. Personality change and psychotic behavior can manifest with increasing age.

Individuals with later-onset Fabry disease can be male or female. Late-onset Fabry disease presents as the atypical variant form, and growing evidence indicates there may be a significant number of “atypical variants” which are unaccounted for in the world. Females, who inherit an X chromosome containing an a-GAL mutation, may exhibit symptoms later in life, significantly increasing the prevalence of this disease. These patients typically first experience disease symptoms in adulthood, and often have disease symptoms focused on a single organ. For example, many males and females with later-onset Fabry disease have enlargement of the left ventricle of the heart. Later-onset Fabry disease may also present in the form of strokes of unknown cause. As the patients advance in age, the cardiac complications of the disease progress, and can lead to death.

Patients with the milder “cardiac variant” of Fabry diseasenormally have 5-15% of normal a-GAL activity, and present with left ventricular hypertrophy or a cardiomyopathy. These cardiac variant patients remain essentially asymptomatic when their classically affected counterparts are severely compromised. Cardiac variants were found in 1 1% of adult male patients with unexplained left ventricular hypertrophic cardiomyopathy, suggesting that Fabry disease may be more frequent than previously estimated (Nakao et al., N. Engl. J. Med. 1995; 333: 288-293).

There have been several approaches to treatment of Fabry disease. One approved therapy for treating Fabry disease is enzyme replacement therapy (ERT), which typically involves intravenous, infusion of a purified form of the corresponding wild-type protein (Fabrazyme®, Genzyme Corp.). ERT has several drawbacks, however. One of the main complications with enzyme replacement therapy is rapid degradation of the infused protein, which leads to the need for numerous, costly high dose infusions. ERT has several additional caveats, such as difficulties with large-scale generation, purification, and storage of properly folded protein; obtaining glycosylated native protein; generation of an anti-protein immune response; and inability of protein to cross the blood-brain barrier to mitigate central nervous system pathologies (i.e., low bioavailability). In addition, replacement enzyme cannot penetrate the heart or kidney in sufficient amounts to reduce substrate accumulation in the renal podocytes or cardiac myocytes, which figure prominently in Fabry pathology.

Additionally, ERT typically involves intravenous, infusion of a purified form of the corresponding wild-type protein. Two α-Gal A products are currently available for the treatment of Fabry disease: agalsidase alfa (Replagal®, Shire Human Genetic Therapies) and agalsidase beta (Fabrazyme®; Sanofi Genzyme Corporation). While ERT is effective in many settings, the treatment also has limitations. ERT has not been demonstrated to decrease the risk of stroke, cardiac muscle responds slowly, and GL-3 elimination from some of the cell types of the kidneys is limited. Some patients also develop immune reactions to ERT.

Another approach to treating some enzyme deficiencies involves the use of small molecule inhibitors to reduce production of the natural substrate of deficient enzyme proteins, thereby ameliorating the pathology. This “substrate reduction” approach has been specifically described for a class of about 40 related enzyme disorders called lysosomal storage disorders that include glycosphingolipid storage disorders. The small molecule inhibitors proposed for use as therapy are specific for inhibiting the enzymes involved in synthesis of glycolipids, reducing the amount of cellular glycolipid that needs to be broken down by the deficient enzyme.

A third approach to treating Fabry disease has been treatment with what are called pharmacological chaperones (PCs). Such PCs include small molecule inhibitors of α-Gal A, which can bind to the α-Gal A to increase the stability of both mutant enzyme and the corresponding wild type.

Accordingly, there remains a need for therapies for the treatment of Fabry disease.

### SUMMARY

Various aspects of the present invention relate to the treatment of Fabry disease.

One aspect of the present invention pertains to a method of treatment of Fabry disease in a human patient in need thereof. In one or more embodiments, the method comprises administering to the patient a formulation. In some embodiments, the formulation comprises a therapeutically effective dose of migalastat or a salt thereof. In some embodiments, the patient is a pediatric patient. In some embodiments, the patient has an age in a range of from about 2 year to about <18 year. In some embodiments, the patient has a weight in a range of from about <15 kg to about ≥50 kg. In some embodiments, the therapeutically effective dose of migalastat or a salt thereof is in a range of from about 15 mg to about 150 mg every other day. In some embodiments, the therapeutically effective dose of migalastat hydrochloride is in a range of from about 25 mg to about 150 mg every other day. In some embodiments, the therapeutically effective dose of migalastat FBE is in a range of from about 15 mg to about 123 mg every other day.

In one or more embodiments, the patient has an age in a range of from 12 to <18. In some embodiments, the patient has a weight of about ≥25 kg. In some embodiments, 10 the therapeutically effective dose of migalastat hydrochloride is in a range of from about 80 mg to about 150 mg every other day. In one or more embodiments, the patient has a weight of about ≥45 kg. In some embodiments, the therapeutically effective dose of migalastat hydrochloride is about 150 mg every other day. In some embodiments, the therapeutically effective dose of migalastat FBE is about 123 mg every other day.

In one or more embodiments, the patient has an age in a range of from about 6 year to about <12 year. In some embodiments, the patient has a weight of about ≥25 kg. In some embodiments, the therapeutically effective dose of migalastat hydrochloride is in a range of from about 80 mg to about 150 mg every other day.

In one or more embodiments, the patient has an age in a range of from about 2 year to about <6 year. In some embodiments, the patient has a weight of about <35 kg. In some embodiments, the therapeutically effective dose of migalastat hydrochloride is in a range of from about 40 mg to about 80 mg every other day.

In one or more embodiments, the patient has an eGFR of about ≥60 mL/min/1.73 m².

In one or more embodiments, the migalastat or salt thereof enhances or prolongs α-galactosidase A activity.

In one or more embodiments, the formulation comprises an oral dosage form. In some embodiments, the oral dosage form comprises a tablet, a capsule or a solution.

In one or more embodiments, the patient is male.

In one or more embodiments, the patient is female.

In one or more embodiments, the patient is an ERT-naïve patient.

In one or more embodiments, the patient is an ERT-experienced patient, who has stopped ERT for at least 14 days.

In one or more embodiments, the patient has a HEK assay amenable mutation in α-galactosidase A. In one or more embodiments, the mutation is disclosed in a pharmacological reference table. In one or more embodiments, the pharmacological reference table is provided in a product label for a migalastat product approved for the treatment of Fabry disease. In one or more embodiments, the pharmacological reference table is provided in a product label for GALAFOLD®. In one or more embodiments, the pharmacological reference table is provided at a website. In one or more embodiments, the website is one or more of www.galafoldamenabilitytable.com or www.fabrygenevariantsearch.com.

### BRIEF DESCRIPTION OF THE DRAWINGS

Further features of the present invention will become apparent from the following written description and the accompanying figures, in which:

FIGS. 1A-E show the full DNA sequence of the human wild-type GLA gene (SEQ ID NO: 1);

FIG. 2 shows the wild-type α-Gal A protein (SEQ ID NO: 2); and

FIG. 3 shows the nucleic acid sequence encoding the wild-type α-Gal A protein (SEQ ID NO: 3).

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

Various aspects of the present invention pertain to the administration of pharmacological chaperones such as migalastat for the treatment of Fabry disease in pediatric and adolescent patients.

### Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

The term “Fabry disease” refers to an X-linked inborn error of glycosphingolipid catabolismdue to deficient lysosomal α-Gal A activity. This defect causes accumulation of the substrate globotriaosylceramide (“GL-3”, also known as Gb3 or ceramide trihexoside) and related glycosphingolipids in vascular endothelial lysosomes of the heart, kidneys, skin, and other tissues. Another substrate of the enzyme is plasma globotriaosylsphingosine (“plasma lyso-Gb₃”).

The term “atypical Fabry disease” refers to patients with primarily cardiac manifestations of the α-Gal A deficiency, namely progressive GL-3 accumulation in myocardial cells that leads to significant enlargement of the heart, particularly the left ventricle.

A “carrier” is a female who has one X chromosome with a defective α-Gal A gene and one X chromosome with the normal gene and in whom X chromosome inactivation of the normal allele is present in one or more cell types. A carrier is often diagnosed with Fabry disease.

A “patient” refers to a subject who has been diagnosed with or is suspected of having a particular disease. The patient may be human or animal.

A “Fabry patient” refers to an individual who has been diagnosed with or suspected of having Fabry disease and has a mutated α-Gal A as defined further below. Characteristic markers of Fabry disease can occur in male hemizygotes and female carriers with the same prevalence, although females typically are less severely affected.

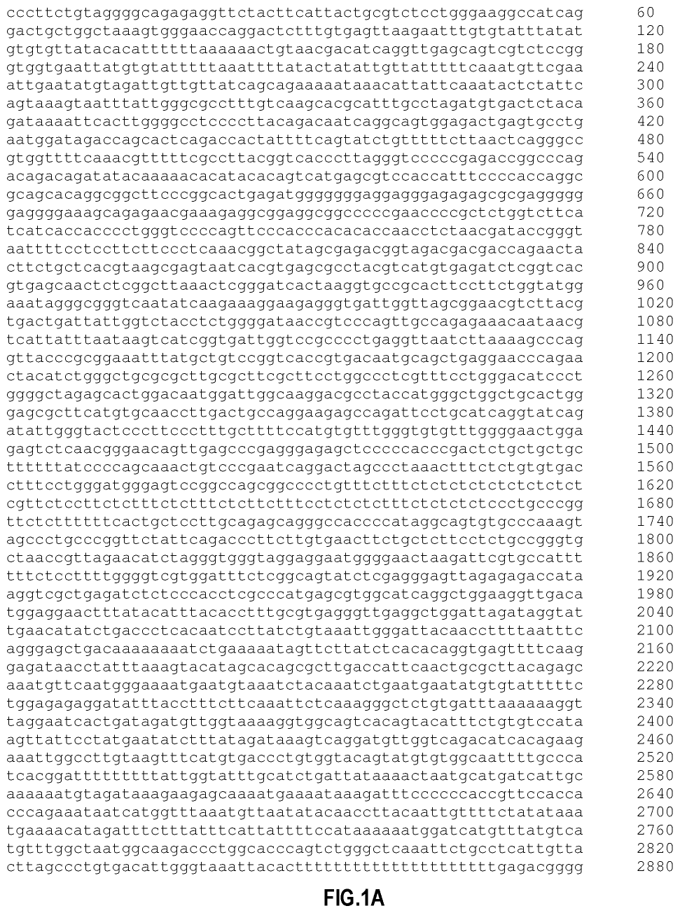

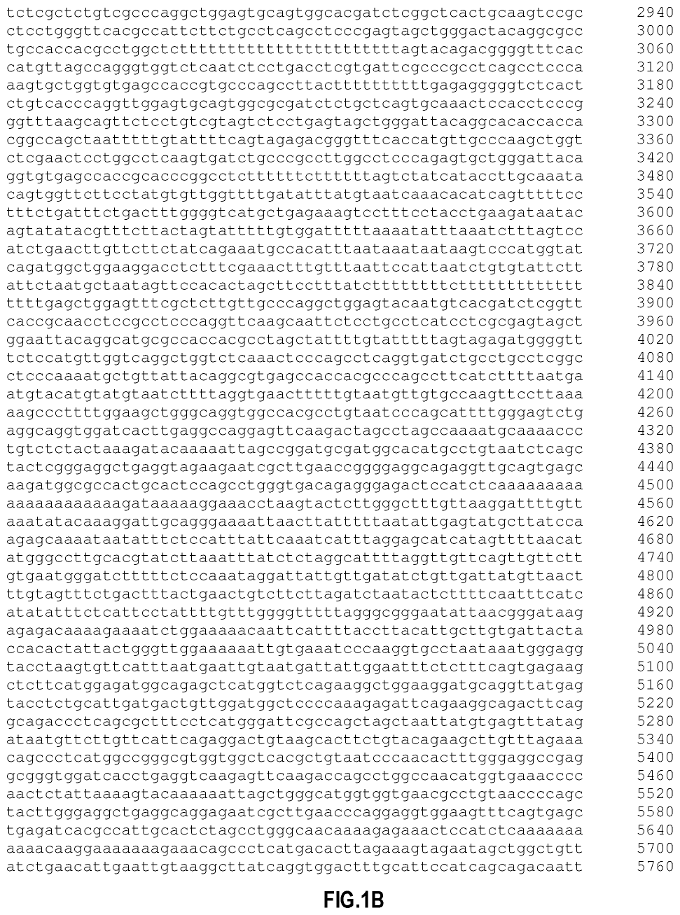

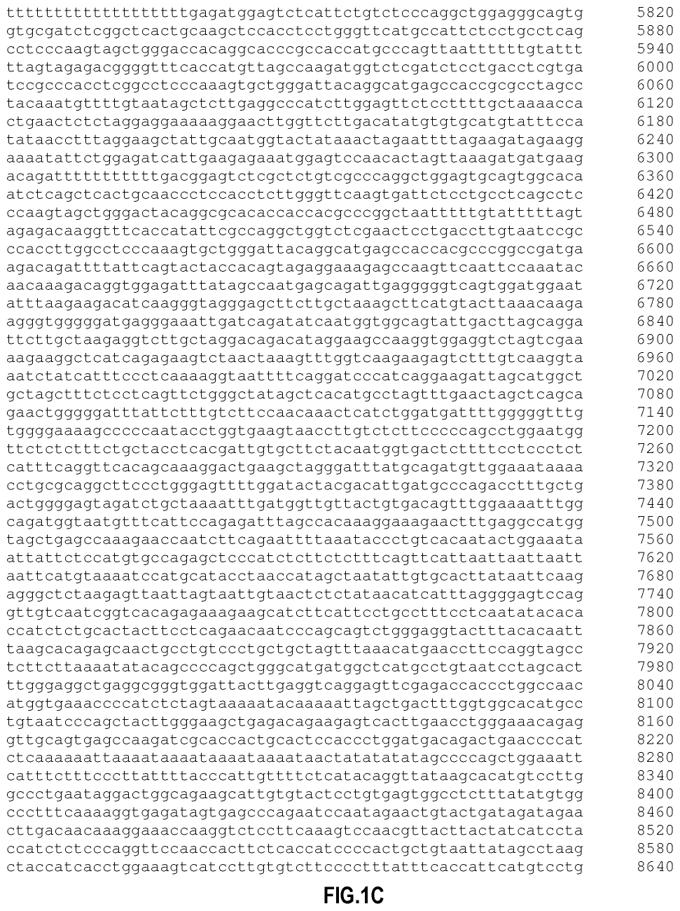

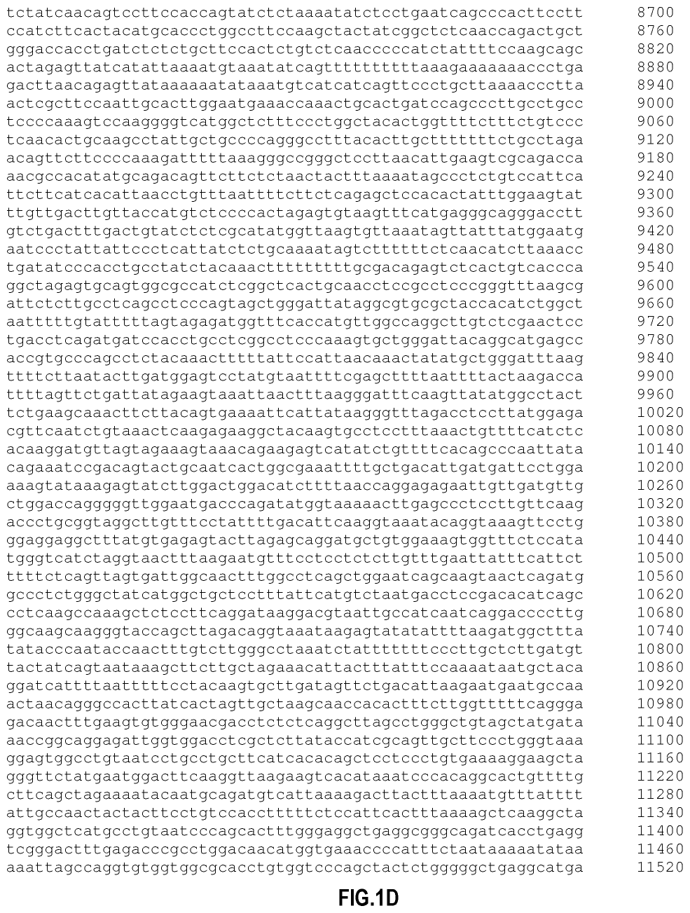

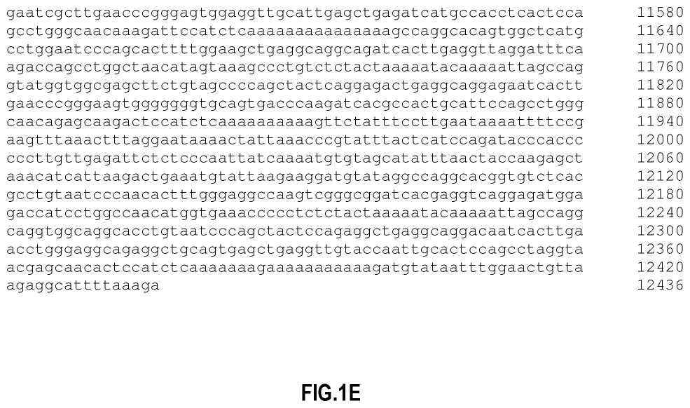

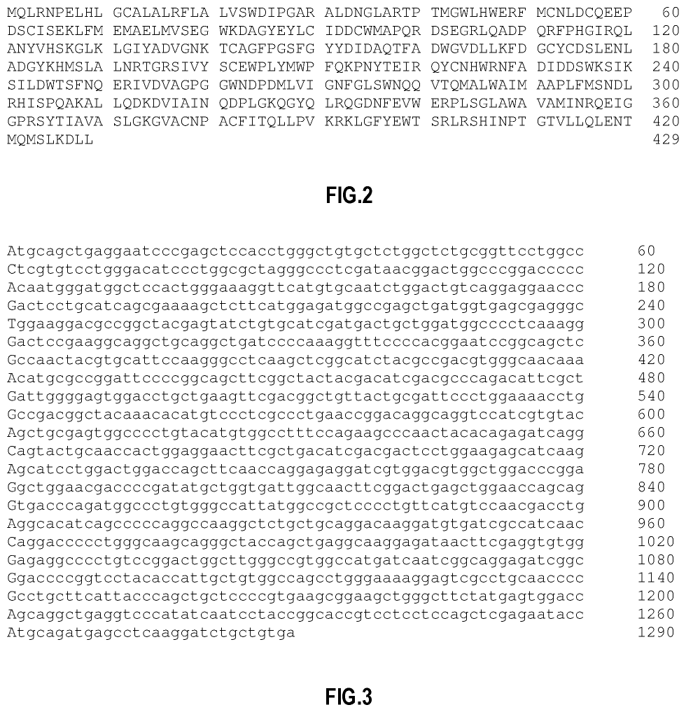

Human α-galactosidase A (α-Gal A) refers to an enzyme encoded by the human GLA gene. The full DNA sequence of α-Gal A, including introns and exons, is available in GenBank Accession No. X14448.1 and shown in FIG. 1A-E (SEQ ID NO: 1). The human α-Gal A enzyme consists of 429 amino acids and is available in GenBank Accession Nos. X14448.1 and U78027.1 and shown in FIG. 2 (SEQ ID NO: 2). The nucleic acid sequence that only includes the coding regions (i.e. exons) of SEQ ID NO: 1 is shown in FIG. 3 (SEQ ID NO: 3).

The term “mutant protein” includes a protein which has a mutation in the gene encoding the protein which results in the inability of the protein to achieve a stable conformation under the conditions normally present in the endoplasmic reticulum (ER). The failure to achieve a stable conformation results in a substantial amount of the enzyme being degraded, rather than being transported to the lysosome. Such a mutation is sometimes called a “conformational mutant.” Such mutations include, but are not limited to, missense mutations, and in-frame small deletions and insertions.

As used herein in one embodiment, the term “mutant α-Gal A” includes an α-Gal A which has a mutation in the gene encoding α-Gal A which results in the inability of the enzyme to achieve a stable conformation under the conditions normally present in the ER. The failure to achieve a stable conformation results in a substantial amount of the enzyme being degraded, rather than being transported to the lysosome.

As used herein, the term “pharmacological chaperone” (“PC”) or “specific pharmacological chaperone” (“SPC”) refers to any molecule including a small molecule, protein, peptide, nucleic acid, carbohydrate, etc. that specifically binds to a protein and has one or more of the following effects: (i) enhances the formation of a stable molecular conformation of the protein; (ii) induces trafficking of the protein from the ER to another cellular location, preferably a native cellular location, i.e., prevents ER-associated degradation of the protein; (iii) prevents aggregation of misfolded proteins; and/or (iv) restores or enhances at least partial wild-type function and/or activity to the protein. A compound that specifically binds to e.g., α-Gal A, means that it binds to and exerts a chaperone effect on the enzyme and not a generic group of related or unrelated enzymes. More specifically, this term does not refer to endogenous chaperones, such as BiP, or to non-specific agents which have demonstrated non-specific chaperone activity against various proteins, such as glycerol, DMSO or deuterated water, i.e., chemical chaperones. In one or more embodiments of the present invention, the PC may be a reversible competitive inhibitor. In one embodiment, the PC is migalastat or a salt thereof. In another embodiment, the PC is migalastat free base (e.g., 123 mg of migalastat free base). In yet another embodiment, the PC is a salt of migalastat (e.g., 150 mg of migalastat HCl).

A “competitive inhibitor” of an enzyme can refer to a compound which structurally resembles the chemical structure and molecular geometry of the enzyme substrate to bind the enzyme in approximately the same location as the substrate. Thus, the inhibitor competes for the same active site as the substrate molecule, thus increasing the Km. Competitive inhibition is usually reversible if sufficient substrate molecules are available to displace the inhibitor, i.e., competitive inhibitors can bind reversibly. Therefore, the amount of enzyme inhibition depends upon the inhibitor concentration, substrate concentration, and the relative affinities of the inhibitor and substrate for the active site.

As used herein, the term “specifically binds” refers to the interaction of a pharmacological chaperone with a protein such as α-Gal A, specifically, an interaction with amino acid residues of the protein that directly participate in contacting the pharmacological chaperone. A pharmacological chaperone specifically binds a target protein, e.g., α-Gal A, to exert a chaperone effect on the protein and not a generic group of related or unrelated proteins. The amino acid residues of a protein that interact with any given pharmacological chaperone may or may not be within the protein's “active site.” Specific binding can be evaluated through routine binding assays or through structural studies, e.g., co-crystallization, NMR, and the like. The active site for α-Gal A is the substrate binding site.

“Deficient α-Gal A activity” refers to α-Gal A activity in cells from a patient which is below the normal range as compared (using the same methods) to the activity in normal individuals not having or suspected of having Fabry or any other disease (especially a blood disease).

As used herein, the terms “enhance α-Gal A activity” or “increase α-Gal A activity” refer to increasing the amount of α-Gal A that adopts a stable conformation in a cell contacted with a pharmacological chaperone specific for the α-Gal A, relative to the amount in a cell (preferably of the same cell-type or the same cell, e.g., at an earlier time) not contacted with the pharmacological chaperone specific for the α-Gal A. This term also refers to increasing the trafficking of α-Gal A to the lysosome in a cell contacted with a pharmacological chaperone specific for the α-Gal A, relative to the trafficking of α-Gal A not contacted with the pharmacological chaperone specific for the protein. These terms refer to both wild-type and mutant α-Gal A. In one embodiment, the increase in the amount of α-Gal A in the cell is measured by measuring the hydrolysis of an artificial substrate in lysates from cells that have been treated with the PC. An increase in hydrolysis is indicative of increased α-Gal A activity.

The term “α-Gal A activity” refers to the normal physiological function of a wild-type α-Gal A in a cell. For example, α-Gal A activity includes hydrolysis of GL-3.

A “responder” is an individual diagnosed with or suspected of having a lysosomal storage disorder (LSD), such, for example Fabry disease, whose cells exhibit sufficiently increased α-Gal A activity, respectively, and/or amelioration of symptoms or enhancement in surrogate markers, in response to contact with a PC. Non-limiting examples of enhancements in surrogate markers for Fabry are lyso-GB3 and those disclosed in US Patent Application Publication No. U.S. 2010/0113517, which is hereby incorporated by reference in its entirety.

Non-limiting examples of improvements in surrogate markers for Fabry disease disclosed in U.S. 2010/0113517 include increases in α-Gal A levels or activity in cells (e.g., fibroblasts) and tissue; reductions in of GL-3 accumulation; decreased plasma concentrations of homocysteine and vascular cell adhesion molecule-1 (VCAM-1); decreased GL-3 accumulation within myocardial cells and valvular fibrocytes; reduction in plasma lyso-Gb3; reduction in cardiac hypertrophy (especially of the left ventricle), amelioration of valvular insufficiency, and arrhythmias; amelioration of proteinuria; decreased urinary concentrations of lipids such as CTH, lactosylceramide, ceramide, and increased urinary concentrations of glucosylceramide and sphingomyelin; the absence of laminated inclusion bodies (Zebra bodies) in glomerular epithelial cells; improvements in renal function; mitigation of hypohidrosis; the absence of angiokeratomas; and improvements in hearing abnormalities such as high frequency sensorineural hearing loss progressive hearing loss, sudden deafness, or tinnitus. Improvements in neurological symptoms include prevention of transient ischemic attack (TIA) or stroke; and amelioration of neuropathic pain manifesting itself as acroparaesthesia (burning or tingling in extremities). Another type of clinical marker that can be assessed for Fabry disease is the prevalence of deleterious cardiovascular manifestations. Common cardiac-related signs and symptoms of Fabry disease include left ventricular hypertrophy, valvular disease (especially mitral valve prolapse and/or regurgitation), premature coronary artery disease, angina, myocardial infarction, conduction abnormalities, arrhythmias, congestive heart failure.

The dose that achieves one or more of the aforementioned responses is a “therapeutically effective dose.”

The phrase “pharmaceutically acceptable” refers to molecular entities and compositions that are physiologically tolerable and do not typically produce untoward reactions when administered to a human. In some embodiments, as used herein, the term “pharmaceutically acceptable” means approved by a regulatory agency of the Federal or a state government or listed in the U.S. Pharmacopoeia or other generally recognized pharmacopoeia for use in animals, and more particularly in humans. The term “carrier” in reference to a pharmaceutical carrier refers to a diluent, adjuvant, excipient, or vehicle with which the compound is administered. Such pharmaceutical carriers can be sterile liquids, such as water and oils. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition, or other editions.

As used herein, the term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes a PCR product, an mRNA band on a gel, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acids include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “enzyme replacement therapy” or “ERT” refers to the introduction of a non-native, purified enzyme into an individual having a deficiency in such enzyme. The administered protein can be obtained from natural sources or by recombinant expression (as described in greater detail below). The term also refers to the introduction of a purified enzyme in an individual otherwise requiring or benefiting from administration of a purified enzyme, e.g., suffering from enzyme insufficiency. The introduced enzyme may be a purified, recombinant enzyme produced in vitro, or protein purified from isolated tissue or fluid, such as, e.g., placenta or animal milk, or from plants.

The term “ERT-naïve patient” refers to a Fabry patient that has never received ERT or has not received ERT for at least 6 months prior to initiating migalastat therapy.

The term “ERT-experienced patient” refers to a Fabry patient that was receiving ERT immediately prior to initiating migalastat therapy. In some embodiments, the ERT-experienced patient has received at least 12 months of ERT immediately prior to initiating migalastat therapy.

As used herein, the term “free base equivalent” or “FBE” refers to the amount of migalastat present in the migalastat or salt thereof. In other words, the term “FBE” means either an amount of migalastat free base, or the equivalent amount of migalastat free base that is provided by a salt of migalastat. For example, due to the weight of the hydrochloride salt, 150 mg of migalastat hydrochloride only provides as much migalastat as 123 mg of the free base form of migalastat. Other salts are expected to have different conversion factors, depending on the molecular weight of the salt.

The term “migalastat” encompasses migalastat free base or a pharmaceutically acceptable salt thereof (e.g., migalastat HCl), unless specifically indicated to the contrary.

The terms “mutation” and “variant” (e.g., as in “amenable mutation or variant”) refer to a change in the nucleotide sequence of a gene or a chromosome. The two terms referred herein are typically used together—e.g., as in “mutation or variant”—referring to the change in nucleotide sequence stated in the previous sentence. If only one of the two terms is recited for some reason, the missing term was intended to be included and one should understand as such. Furthermore, the terms “amenable mutation” and “amenable variant” refer to a mutation or variant that is amenable to PC therapy, e.g., a mutation that is amenable to migalastat therapy. A particular type of amenable mutation or variant is a “HEK assay amenable mutation or variant”, which is a mutation or variant that is determined to be amenable to migalastat therapy according to the criteria in the in vitro HEK assay described herein and in U.S. Pat. No. 8,592,362, which is hereby incorporated by reference in its entirety.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 10-or 5-fold, and more preferably within 2-fold of a given value. Numerical quantities given herein are approximate unless stated otherwise, meaning that the term “about” or “approximately” can be inferred when not expressly stated.

### Fabry Disease

Fabry disease is a rare, progressive and devastating X-linked lysosomal storage disorder (LSD). Mutations in the GLA gene result in a deficiency of the lysosomal enzyme, α-Gal A, which is required for glycosphingolipid metabolism. Beginning early in life, the reduction in α-Gal A activity results in an accumulation of glycosphingolipids, including GL-3 and plasma lyso-Gb3, and leads to the symptoms and life-limiting sequelae of Fabry disease, including pain, gastrointestinal symptoms, renal failure, cardiomyopathy, cerebrovascular events, and early mortality. Early initiation of therapy and lifelong treatment provide an opportunity to slow disease progression and prolong life expectancy.

Fabry disease encompasses a spectrum of disease severity and age of onset, although it has traditionally been divided into 2 main phenotypes, “classic” and “late-onset”. The classic phenotype has been ascribed primarily to males with undetectable to low α-Gal A activity and earlier onset of renal, cardiac and/or cerebrovascular manifestations. The late-onset phenotype has been ascribed primarily to males with higher residual α-Gal A activity and later onset of these disease manifestations. Heterozygous female carriers typically express the late-onset phenotype but depending on the pattern of X-chromosome inactivation may also display the classic phenotype.

More than 1,000 Fabry disease-causing GLA mutations have been identified. The GLA mutation includes but not limited to missense, nonsense, and splicing mutations, in addition to small deletions and insertions, and larger gene rearrangements. Approximately 60% are missense mutations, resulting in single amino acid substitutions in the α-Gal A enzyme. Missense GLA mutations often result in the production of abnormally folded and unstable forms of α-Gal A and the majority are associated with the classic phenotype. Normal cellular quality control mechanisms in the ER block the transit of these abnormal proteins to lysosomes and target them for premature degradation and elimination. Many missense mutant forms are targets for migalastat, an α-Gal A-specific pharmacological chaperone.

The clinical manifestations of Fabry disease span a broad spectrum of severity and roughly correlate with a patient's residual α-Gal A levels. The majority of currently treated patients are referred to as classic Fabry patients, most of whom are males. These patients experience disease of various organs, including the kidneys, heart and brain, with disease symptoms first appearing in adolescence and typically progressing in severity until death in the fourth or fifth decade of life. A number of recent studies suggest that there are a large number of undiagnosed males and females that have a range of Fabry disease symptoms, such as impaired cardiac or renal function and strokes, that usually first appear in adulthood. Individuals with this type of Fabry disease, referred to as later-onset Fabry disease, tend to have higher residual α-Gal A levels than classic Fabry patients. Individuals with later-onset Fabry disease typically first experience disease symptoms in adulthood, and often have disease symptoms focused on a single organ, such as enlargement of the left ventricle or progressive kidney failure. In addition, later-onset Fabry disease may also present in the form of strokes of unknown cause.

Because Fabry disease is rare, involves multiple organs, has a wide age range of onset, and is heterogeneous, proper diagnosis is a challenge. For example, Fabry patients have progressive kidney impairment, and untreated patients exhibit end-stage renal impairment by the fifth decade of life. Deficiency in α-Gal A activity leads to accumulation of globotriaosylceramide (Gb3) and related glycosphingolipids in many cell types including cells in the kidney. Gb3 accumulates in podocytes, epithelial cells and the tubular cells of the distal tubule and loop of Henle. Impairment in kidney function can manifest as proteinuria and reduced glomerular filtration rate.

Furthermore, awareness is low among health care professionals and misdiagnoses are frequent. Diagnosis of Fabry disease is most often confirmed on the basis of decreased α-Gal A activity in plasma or peripheral leukocytes (WBCs) once a patient is symptomatic, coupled with mutational analysis. In females, diagnosis is even more challenging since the enzymatic identification of carrier females is less reliable due to random X-chromosomal inactivation in some cells of carriers. For example, some obligate carriers (daughters of classically affected males) have α-Gal A enzyme activities ranging from normal to very low activities. Since carriers can have normal α-Gal A enzyme activity in leukocytes, only the identification of an α-Gal A mutation by genetic testing provides precise carrier identification and/or diagnosis.

In one or more embodiments, mutant forms of α-Gal A are considered to be amenable to migalastat are defined as showing a relative increase (+10 μM migalastat) of ≥1.20-fold and an absolute increase (+10 μM migalastat) of ≥ 3.0% wild-type (WT) when the mutant form of α-Gal A is expressed in HEK-293 cells (referred to as the “HEK assay”) according to Good Laboratory Practice (GLP)-validated in vitro assay (GLP HEK or Migalastat Amenability Assay). Such mutations are also referred to herein as “HEK assay amenable” mutations.

Previous screening methods have been provided that assess enzyme enhancement prior to the initiation of treatment. For example, an assay using HEK-293 cells has been utilized in clinical trials to predict whether a given mutation will be responsive to pharmacological chaperone (e.g., migalastat) treatment. In this assay, cDNA constructs are created. The corresponding α-Gal A mutant forms are transiently expressed in HEK-293 cells. Cells are then incubated±migalastat (17 nM to 1 mM) for 4 to 5 days. After, α-Gal A levels are measured in cell lysates using a synthetic fluorogenic substrate (4-MU-α-Gal) or by western blot. This has been done for known disease-causing missense or small in-frame insertion/deletion mutations. Mutations that have previously been identified as responsive to a PC (e.g., migalastat) using these methods are listed in U.S. Pat. No. 8,592,362, which is hereby incorporated by reference in its entirety.

### Pharmacological Chaperones

The binding of small molecule inhibitors of enzymes associated with LSDs can increase the stability of both mutant enzyme and the corresponding wild-type enzyme (see U.S. Pat. Nos. 6,274,597; 6,583,158; 6,589,964; 6,599,919; 6,916,829, and 7,141,582 all incorporated herein by reference). In particular, administration of small molecule derivatives of glucose and galactose, which are specific, selective competitive inhibitors for several target lysosomal enzymes, effectively increased the stability of the enzymes in cells in vitro and, thus, increased trafficking of the enzymes to the lysosome. Thus, by increasing the amount of enzyme in the lysosome, hydrolysis of the enzyme substrates is expected to increase. The original theory behind this strategy was as follows: since the mutant enzyme protein is unstable in the ER (Ishii et al., Biochem. Biophys. Res. Comm. 1996; 220: 812-815), the enzyme protein is retarded in the normal transport pathway (ER→Golgi apparatus→endosomes→lysosome) and prematurely degraded. Therefore, a compound which binds to and increases the stability of a mutant enzyme, may serve as a “chaperone” for the enzyme and increase the amount that can exit the ER and move to the lysosomes. In addition, because the folding and trafficking of some wild-type proteins is incomplete, with up to 70% of some wild-type proteins being degraded in some instances prior to reaching their final cellular location, the chaperones can be used to stabilize wild-type enzymes and increase the amount of enzyme which can exit the ER and be trafficked to lysosomes.

In one or more embodiments, the pharmacological chaperone comprises migalastat or a thereof. The compound migalastat, also known as 1-deoxygalactonojirimycin (1-DGJ) or (2R,3S,4R,5S)-2-(hydroxymethyl) piperdine-3,4,5-triol is a compound having the following chemical formula:

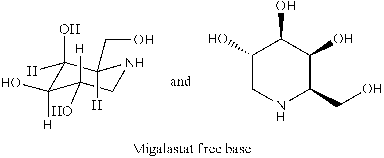 [SMILES: OCC1NCC(O)C(O)C1O ; OC[C@H]1NC[C@H](O)[C@@H](O)[C@H]1O]

As discussed herein, pharmaceutically acceptable salts of migalastat may also be used in the present invention. When a salt of migalastat is used, the dosage of the salt will be adjusted so that the dose of migalastat received by the patient is equivalent to the amount which would have been received had the migalastat free base been used. One example of a pharmaceutically acceptable salt of migalastat is migalastat HCl:

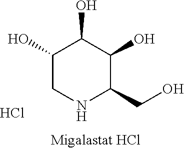 [SMILES: OC[C@H]1NC[C@H](O)[C@@H](O)[C@H]1O ; Cl]

Migalastat is a low molecular weight iminosugar and is an analogue of the terminal galactose of GL-3. In vitro and in vivo pharmacologic studies have demonstrated that migalastat acts as a pharmacological chaperone, selectively and reversibly binding, with high affinity, to the active site of wild-type α-Gal A and specific mutant forms of α-Gal A, the genotypes of which are referred to as HEK assay amenable mutations. Migalastat binding stabilizes these mutant forms of α-Gal A in the endoplasmic reticulum facilitating their proper trafficking to lysosomes where dissociation of migalastat allows α-Gal A to reduce the level of GL-3 and other substrates. Approximately 30-50% of patients with Fabry disease have HEK assay amenable mutations; the majority of which are associated with the classic phenotype of the disease.

HEK assay amenable mutations include at least those mutations listed in a pharmacological reference table (e.g., the ones recited in the U.S. or International Product labels for a migalastat product such as GALAFOLD®). As used herein, “pharmacological reference table” refers to any publicly accessible written or electronic record, included in either the product label within the packaging of a migalastat product (e.g., GALAFOLD®) or in a website accessible by health care providers, that conveys whether a particular mutation or variant is responsive to migalastat (e.g., GALAFOLD®) PC therapy, and is not necessarily limited to written records presented in tabular form. In one embodiment of the present invention, a “pharmacological reference table” thus refers to any depository of information that includes one or more amenable mutations or variants. An exemplary pharmacological reference table for HEK assay amenable mutations can be found in the summary of product characteristics and/or prescribing information for GALAFOLD® in various countries in which GALAFOLD® is approved for use, or at a website such as www.galafoldamenabilitytable.com or www.fabrygenevariantsearch.com, each of which is hereby incorporated by reference in its entirety.

Although the vast majority of α-GAL mutations are missense mutations, with most being outside the catalytic site, it difficult to predict which mutations result in an unstable enzyme that could be “rescued” by a pharmacological chaperone (PC) which stabilizes the enzyme, and which ones cannot be stabilized using a PC.

An exemplary pharmacological reference table for HEK assay amenable mutations is provided in Table 1 below. In one or more embodiments, if a double mutation is present on the same chromosome (males and females), that patient is considered HEK assay amenable if the double mutation is present in one entry in Table 1 (e.g., D55V/Q57L). In some embodiments, if a double mutation is present on different chromosomes (only in females) that patient is considered HEK assay amenable if either one of the individual mutations is present in Table 1.

TABLE 1
HEK Amenable Mutations

| Nucleotide change | Nucleotide change | Protein sequence change |
|---|---|---|
| c.7C > G | c.C7G | L3V |
| c.8T > C | c.T8C | L3P |
| c.[11G > T; 620A > C] | c.G11T/A620C | R4M/Y207S |
| c.13A > G | c.A13G | N5D |
| c.15C > G | c.C15G | N5K |
| c.16C > A | c.C16A | P6T |
| c.16C > T | c.C16T | P6S |
| c.17C > A | c.C17A | P6Q |
| c.17C > G | c.C17G | P6R |
| c.17C > T | c.C17T | P6L |
| c.19G > A | c.G19A | E7K |
| c.20A > T | c.A20T | E7V |
| c.21A > T | c.A21T | E7D |
| c.22C > A | c.C22A | L8I |
| c.23T > A | c.T23A | L8Q |
| c.23T > C | c.T23C | L8P |
| c.25C > T | c.C25T | H9Y |
| c.26A > G | c.A26G | H9R |
| c.26A > T | c.A26T | H9L |
| c.27T > A | c.T27A | H9Q |
| c.28C > A | c.C28A | L10M |
| c.28C > G | c.C28G | L10V |
| c.29T > A | c.T29A | L10Q |
| c.29T > C | c.T29C | L10P |
| c.29T > G | c.T29G | L10R |
| c.31G > A | c.G31A | G11S |
| c.31G > C | c.G31C | G11R |
| c.31G > T | c.G31T | G11C |
| c.32G > A | c.G32A | G11D |
| c.32G > T | c.G32T | G11V |
| c.34T > A | c.T34A | C12S |
| c.34T > C | c.T34C | C12R |
| c.34T > G | c.T34G | C12G |
| c.35G > A | c.G35A | C12Y |
| c.37G > A | c.G37A | A13T |
| c.37G > C | c.G37C | A13P |
| c.38C > A | c.C38A | A13E |
| c.38C > G | c.C38G | A13G |
| c.40C > G | c.C40G | L14V |
| c.40C > T | c.C40T | L14F |
| c.41T > A | c.T41A | L14H |
| c.43G > A | c.G43A | A15T |
| c.44C > G | c.C44G | A15G |
| c.49C > A | c.C49A | R17S |
| c.49C > G | c.C49G | R17G |
| c.49C > T | c.C49T | R17C |
| c.50G > A | c.G50A | R17H |
| c.50G > C | c.G50C | R17P |
| c.52T > A | c.T52A | F18I |
| c.53T > G | c.T53G | F18C |
| c.54C > G | c.C54G | F18L |
| c.58G > C | c.G58C | A20P |
| c.59C > A | c.C59A | A20D |
| c.59C > G | c.C59G | A20G |
| c.62T > A | c.T62A | L21H |
| c.64G > A | c.G64A | V22I |
| c.64G > C | c.G64C | V22L |
| c.64G > T | c.G64T | V22F |
| c.65T > C | c.T65C | V22A |
| c.65T > G | c.T65G | V22G |
| c.67T > A | c.T67A | S23T |
| c.67T > C | c.T67C | S23P |
| c.70T > C or c.70T > A | c.T70C or c.T70A | W24R |
| c.70T > G | c.T70G | W24G |
| c.71G > C | c.G71C | W24S |
| c.72G > C or c.72G > T | c.G72C or c.G72T | W24C |
| c.73G > C | c.G73C | D25H |
| c.77T > A | c.T77A | I26N |
| c.79C > A | c.C79A | P27T |
| c.79C > G | c.C79G | P27A |
| c.79C > T | c.C79T | P27S |
| c.80C > T | c.C80T | P27L |
| c.82G > C | c.G82C | G28R |
| c.82G > T | c.G82T | G28W |
| c.83G > A | c.G83A | G28E |
| c.85G > C | c.G85C | A29P |
| c.86C > A | c.C86A | A29D |
| c.86C > G | c.C86G | A29G |
| c.86C > T | c.C86T | A29V |
| c.88A > G | c.A88G | R30G |
| c.94C > A | c.C94A | L32M |
| c.94C > G | c.C94G | L32V |
| c.95T > A | c.T95A | L32Q |
| c.95T > C | c.T95C | L32P |
| c.95T > G | c.T95G | L32R |
| c.97G > C | c.G97C | D33H |
| c.97G > T | c.G97T | D33Y |
| c.98A > C | c.A98C | D33A |
| c.98A > G | c.A98G | D33G |
| c.98A > T | c.A98T | D33V |
| c.99C > G | c.C99G | D33E |
| c.100A > C | c.A100C | N34H |
| c.100A > G | c.A100G | N34D |
| c.101A > C | c.A101C | N34T |
| c.101A > G | c.A101G | N34S |
| c.102T > G or c.102T > A | c.T102G or c.T102A | N34K |
| c.103G > C or c.103G > A | c.G103C or c.G103A | G35R |
| c.104G > A | c.G104A | G35E |
| c.104G > C | c.G104C | G35A |
| c.104G > T | c.G104T | G35V |
| c.106T > A | c.T106A | L36M |
| c.106T > G | c.T106G | L36V |
| c.107T > C | c.T107C | L36S |
| c.107T > G | c.T107G | L36W |
| c.108G > C or c.108G > T | c.G108C or c.G108T | L36F |
| c.109G > A | c.G109A | A37T |
| c.109G > T | c.G109T | A37S |
| c.110C > A | c.C110A | A37E |
| c.110C > G | c.C110G | A37G |
| c.110C > T | c.C110T | A37V |
| c.112A > G | c.A112G | R38G |
| c.112A > T | c.A112T | R38W |
| c.113G > T | c.G113T | R38M |
| c.114G > C | c.G114C | R38S |
| c.115A > G | c.A115G | T39A |
| c.115A > T | c.A115T | T39S |
| c.116C > A | c.C116A | T39K |
| c.116C > G | c.C116G | T39R |
| c.116C > T | c.C116T | T39M |
| c.121A > G | c.A121G | T41A |
| c.122C > A | c.C122A | T41N |
| c.122C > G | c.C122G | T41S |
| c.122C > T | c.C122T | T41I |
| c.124A > C or c.124A > T | c.A124C or c.A124T | M42L |
| c.124A > G | c.A124G | M42V |
| c.125T > A | c.T125A | M42K |
| c.125T > C | c.T125C | M42T |
| c.125T > G | c.T125G | M42R |
| c.126G > A or c.126G > C | c.G126A or c.G126C | M42I |
| or c.126G > T | or c.G126T | |
| c.128G > C | c.G128C | G43A |
| c.133C > A | c.C133A | L45M |
| c.133C > G | c.C133G | L45V |
| c.136C > A | c.C136A | H46N |
| c.136C > G | c.C136G | H46D |
| c.137A > C | c.A137C | H46P |
| c.138C > G | c.C138G | H46Q |
| c.142G > C | c.G142C | E48Q |
| c.143A > C | c.A143C | E48A |
| c.149T > A | c.T149A | F50Y |
| c.151A > G | c.A151G | M51V |
| c.152T > A | c.T152A | M51K |
| c.152T > C | c.T152C | M51T |
| c.152T > G | c.T152G | M51R |
| c.153G > A or c.153G > T | c.G153A or c.G153T | M51I |
| or c.153G > C | or c.G153C | |
| c.157A > C | c.A157C | N53H |
| c.[157A > C; 158A > T] | c.A157C/A158T | N53L |
| c.157A > G | c.A157G | N53D |
| c.157A > T | c.A157T | N53Y |
| c.158A > C | c.A158C | N53T |
| c.158A > G | c.A158G | N53S |
| c.158A > T | c.A158T | N53I |
| c.159C > G or c.159C > A | c.C159G or c.C159A | N53K |
| c.160C > G | c.C160G | L54V |
| c.160C > T | c.C160T | L54F |
| c.161T > A | c.T161A | L54H |
| c.161T > C | c.T161C | L54P |
| c.161T > G | c.T161G | L54R |
| c.163G > C | c.G163C | D55H |
| c.163G > T | c.G163T | D55Y |
| c.164A > C | c.A164C | D55A |
| c.164A > G | c.A164G | D55G |
| c.164A > T | c.A164T | D55V |
| c.[164A > T; 170A > T] | c.A164T/A170T | D55V/Q57L |
| c.165C > G | c.C165G | D55E |
| c.167G > A | c.G167A | C56Y |
| c.167G > T | c.G167T | C56F |
| c.168C > G | c.C168G | C56W |
| c.170A > G | c.A170G | Q57R |
| c.170A > T | c.A170T | Q57L |
| c.172G > A | c.G172A | E58K |
| c.175G > A | c.G175A | E59K |
| c.175G > C | c.G175C | E59Q |
| c.176A > C | c.A176C | E59A |
| c.176A > G | c.A176G | E59G |
| c.176A > T | c.A176T | E59V |
| c.177G > C | c.G177C | E59D |
| c.178C > A | c.C178A | P60T |
| c.178C > G | c.C178G | P60A |
| c.178C > T | c.C178T | P60S |
| c.179C > A | c.C179A | P60Q |
| c.179C > G | c.C179G | P60R |
| c.179C > T | c.C179T | P60L |
| c.182A > T | c.A182T | D61V |
| c.183T > A | c.T183A | D61E |
| c.184_185insTAG | c.184_185insTAG | S62delinsLA |
| c.184T > C | c.T184C | S62P |
| c.184T > G | c.T184G | S62A |
| c.185C > A | c.C185A | S62Y |
| c.185C > G | c.C185G | S62C |
| c.185C > T | c.C185T | S62F |
| c.190A > C | c.A190C | I64L |
| c.190A > G | c.A190G | I64V |
| c.193A > G | c.A193G | S65G |
| c.193A > T | c.A193T | S65C |
| c.195T > A | c.T195A | S65R |
| c.196G > A | c.G196A | E66K |
| c.197A > G | c.A197G | E66G |
| c.197A > T | c.A197T | E66V |
| c.198G > C | c.G198C | E66D |
| c.199A > C | c.A199C | K67Q |
| c.199A > G | c.A199G | K67E |
| c.200A > C | c.A200C | K67T |
| c.200A > T | c.A200T | K67M |
| c.201G > C | c.G201C | K67N |
| c.202C > A | c.C202A | L68I |
| c.205T > A | c.T205A | F69I |
| c.206T > A | c.T206A | F69Y |
| c.207C > A or c.207C > G | c.C207A or c.C207G | F69L |
| c.208A > T | c.A208T | M70L |
| c.209T > A | c.T209A | M70K |
| c.209T > G | c.T209G | M70R |
| c.210G > C | c.G210C | M70I |
| c.211G > C | c.G211C | E71Q |
| c.212A > C | c.A212C | E71A |
| c.212A > G | c.A212G | E71G |
| c.212A > T | c.A212T | E71V |
| c.213G > C | c.G213C | E71D |
| c.214A > G | c.A214G | M72V |
| c.214A > T | c.A214T | M72L |
| c.215T > C | c.T215C | M72T |
| c.216G > A or c.216G > T | c.G216A or c.G216T | M72I |
| or c.216G > C | or c.G216C | |
| c.217G > A | c.G217A | A73T |
| c.217G > T | c.G217T | A73S |
| c.218C > T | c.C218T | A73V |
| c.220G > A | c.G220A | E74K |
| c.221A > G | c.A221G | E74G |
| c.221A > T | c.A221T | E74V |
| c.222G > C | c.G222C | E74D |
| c.223C > T | c.C223T | L75F |
| c.224T > C | c.T224C | L75P |
| c.226A > G | c.A226G | M76V |
| c.227T > C | c.T227C | M76T |
| c.229G > A | c.G229A | V77I |
| c.229G > C | c.G229C | V77L |
| c.232T > C | c.T232C | S78P |
| c.233C > T | c.C233T | S78L |
| c.235G > A | c.G235A | E79K |
| c.235G > C | c.G235C | E79Q |
| c.236A > C | c.A236C | E79A |
| c.236A > G | c.A236G | E79G |
| c.236A > T | c.A236T | E79V |
| c.237A > T | c.A237T | E79D |
| c.238G > A | c.G238A | G80S |
| c.238G > T | c.G238T | G80C |
| c.239G > A | c.G239A | G80D |
| c.239G > C | c.G239C | G80A |
| c.239G > T | c.G239T | G80V |
| c.242G > T | c.G242T | W81L |
| c.244A > G | c.A244G | K82E |
| c.245A > C | c.A245C | K82T |
| c.245A > G | c.A245G | K82R |
| c.245A > T | c.A245T | K82M |
| c.246G > C | c.G246C | K82N |
| c.247G > A | c.G247A | D83N |
| c.248A > C | c.A248C | D83A |
| c.248A > G | c.A248G | D83G |
| c.248A > T | c.A248T | D83V |
| c.249T > A | c.T249A | D83E |
| c.250G > A | c.G250A | A84T |
| c.250G > C | c.G250C | A84P |
| c.250G > T | c.G250T | A84S |
| c.251C > A | c.C251A | A84E |
| c.251C > G | c.C251G | A84G |
| c.251C > T | c.C251T | A84V |
| c.253G > A | c.G253A | G85S |
| c.[253G > A; 254G > A] | c.G253A/G254A | G85N |
| c.[253G > A; 254G > | c.G253A/G254T/T255G | G85M |
| T; 255T > G] | | |
| c.253G > C | c.G253C | G85R |
| c.253G > T | c.G253T | G85C |
| c.254G > A | c.G254A | G85D |
| c.254G > C | c.G254C | G85A |
| c.257A > T | c.A257T | Y86F |
| c.260A > G | c.A260G | E87G |
| c.261G > C or c.261G > T | c.G261C or c.G261T | E87D |
| c.262T > A | c.T262A | Y88N |
| c.262T > C | c.T262C | Y88H |
| c.263A > C | c.A263C | Y88S |
| c.263A > G | c.A263G | Y88C |
| c.265C > G | c.C265G | L89V |
| c.265C > T | c.C265T | L89F |
| c.271A > C | c.A271C | I91L |
| c.271A > T | c.A271T | I91F |
| c.272T > C | c.T272C | I91T |
| c.272T > G | c.T272G | I91S |
| c.273T > G | c.T273G | I91M |
| c.286A > G | c.A286G | M96V |
| c.286A > T | c.A286T | M96L |
| c.287T > C | c.T287C | M96T |
| c.288G > A or c.288G > T | c.G288A or c.G288T | M96I |
| or c.288G > C | or c.G288C | |
| c.289G > A | c.G289A | A97T |
| c.289G > C | c.G289C | A97P |
| c.289G > T | c.G289T | A97S |
| c.290C > A | c.C290A | A97D |
| c.290C > T | c.C290T | A97V |
| c.293C > A | c.C293A | P98H |
| c.293C > G | c.C293G | P98R |
| c.293C > T | c.C293T | P98L |
| c.295C > G | c.C295G | Q99E |
| c.296A > C | c.A296C | Q99P |
| c.296A > G | c.A296G | Q99R |
| c.296A > T | c.A296T | Q99L |
| c.301G > C | c.G301C | D101H |
| c.302A > C | c.A302C | D101A |
| c.302A > G | c.A302G | D101G |
| c.302A > T | c.A302T | D101V |
| c.303T > A | c.T303A | D101E |
| c.304T > A | c.T304A | S102T |
| c.304T > C | c.T304C | S102P |
| c.304T > G | c.T304G | S102A |
| c.305C > T | c.C305T | S102L |
| c.310G > A | c.G310A | G104S |
| c.311G > A | c.G311A | G104D |
| c.311G > C | c.G311C | G104A |
| c.311G > T | c.G311T | G104V |
| c.313A > G | c.A313G | R105G |
| c.314G > A | c.G314A | R105K |
| c.314G > C | c.G314C | R105T |
| c.314G > T | c.G314T | R105I |
| c.316C > A | c.C316A | L106I |
| c.316C > G | c.C316G | L106V |
| c.316C > T | c.C316T | L106F |
| c.317T > A | c.T317A | L106H |
| c.317T > C | c.T317C | L106P |
| c.319C > A | c.C319A | Q107K |
| c.319C > G | c.C319G | Q107E |
| c.320A > G | c.A320G | Q107R |
| c.321G > C | c.G321C | Q107H |
| c.322G > A | c.G322A | A108T |
| c.323C > A | c.C323A | A108E |
| c.323C > T | c.C323T | A108V |
| c.325G > A | c.G325A | D109N |
| c.325G > C | c.G325C | D109H |
| c.325G > T | c.G325T | D109Y |
| c.326A > C | c.A326C | D109A |
| c.326A > G | c.A326G | D109G |
| c.327C > G | c.C327G | D109E |
| c.328C > A | c.C328A | P110T |
| c.334C > G | c.C334G | R112G |
| c.335G > A | c.G335A | R112H |
| c.335G > T | c.G335T | R112L |
| c.337T > A | c.T337A | F113I |
| c.337T > C or c.339T > A | c.T337C or c.T339A | F113L |
| or c.339T > G | or c.T339G | |
| c.337T > G | c.T337G | F113V |
| c.338T > A | c.T338A | F113Y |
| c.341C > T | c.C341T | P114L |
| c.343C > A | c.C343A | H115N |
| c.343C > G | c.C343G | H115D |
| c.346G > C | c.G346C | G116R |
| c.350T > C | c.T350C | I117T |
| c.351T > G | c.T351G | I117M |
| c.352C > T | c.C352T | R118C |
| c.361G > A | c.G361A | A121T |
| c.362C > T | c.C362T | A121V |
| c.367T > A | c.T367A | Y123N |
| c.367T > G | c.T367G | Y123D |
| c.368A > C | c.A368C | Y123S |
| c.368A > G | c.A368G | Y123C |
| c.368A > T | c.A368T | Y123F |
| c.370G > A | c.G370A | V124I |
| c.371T > G | c.T371G | V124G |
| c.373C > A | c.C373A | H125N |
| c.373C > G | c.C373G | H125D |
| c.373C > T | c.C373T | H125Y |
| c.374A > G | c.A374G | H125R |
| c.374A > T | c.A374T | H125L |
| c.376A > G | c.A376G | S126G |
| c.376A > T | c.A376T | S126C |
| c.377G > T | c.G377T | S126I |
| c.379A > G | c.A379G | K127E |
| c.383G > A | c.G383A | G128E |
| c.383G > C | c.G383C | G128A |
| c.385C > G | c.C385G | L129V |
| c.388A > C | c.A388C | K130Q |
| c.389A > T | c.A389T | K130M |
| c.390G > C | c.G390C | K130N |
| c.391C > G | c.C391G | L131V |
| c.397A > C | c.A397C | I133L |
| c.397A > G | c.A397G | I133V |
| c.397A > T | c.A397T | I133F |
| c.398T > C | c.T398C | I133T |
| c.399T > G | c.T399G | I133M |
| c.[399T > G; 434T > C] | c.T399G/T434C | I133M/F145S |
| c.403G > A | c.G403A | A135T |
| c.403G > T | c.G403T | A135S |
| c.404C > A | c.C404A | A135E |
| c.404C > G | c.C404G | A135G |
| c.404C > T | c.C404T | A135V |
| c.406G > A | c.G406A | D136N |
| c.407A > C | c.A407C | D136A |
| c.407A > T | c.A407T | D136V |
| c.408T > A or c.408T > G | c.T408A or c.T408G | D136E |
| c.409G > A | c.G409A | V137I |
| c.409G > C | c.G409C | V137L |
| c.410T > A | c.T410A | V137D |
| c.410T > C | c.T410C | V137A |
| c.410T > G | c.T410G | V137G |
| c.413G > C | c.G413C | G138A |
| c.415A > C | c.A415C | N139H |
| c.415A > T | c.A415T | N139Y |
| c.416A > G | c.A416G | N139S |
| c.416A > T | c.A416T | N139I |
| c.417T > A | c.T417A | N139K |
| c.418A > C | c.A418C | K140Q |
| c.418A > G | c.A418G | K140E |
| c.419A > C | c.A419C | K140T |
| c.419A > G | c.A419G | K140R |
| c.419A > T | c.A419T | K140I |
| c.420A > T | c.A420T | K140N |
| c.421A > T | c.A421T | T141S |
| c.427G > A | c.G427A | A143T |
| c.428C > A | c.C428A | A143E |
| c.428C > G | c.C428G | A143G |
| c.428C > T | c.C428T | A143V |
| c.430G > A | c.G430A | G144S |
| c.430G > C | c.G430C | G144R |
| c.430G > T | c.G430T | G144C |
| c.431G > A | c.G431A | G144D |
| c.431G > C | c.G431C | G144A |
| c.431G > T | c.G431T | G144V |
| c.433T > G | c.T433G | F145V |
| c.434T > A | c.T434A | F145Y |
| c.434T > C | c.T434C | F145S |
| c.434T > G | c.T434G | F145C |
| c.435C > G | c.C435G | F145L |
| c.436C > A | c.C436A | P146T |
| c.436C > G | c.C436G | P146A |
| c.436C > T | c.C436T | P146S |
| c.437C > A | c.C437A | P146H |
| c.437C > G | c.C437G | P146R |
| c.437C > T | c.C437T | P146L |
| c.440G > C | c.G440C | G147A |
| c.442A > G | c.A442G | S148G |
| c.442A > T | c.A442T | S148C |
| c.443G > C | c.G443C | S148T |
| c.446T > G | c.T446G | F149C |
| c.449G > A | c.G449A | G150E |
| c.449G > T | c.G449T | G150V |
| c.451T > G | c.T451G | Y151D |
| c.452A > C | c.A452C | Y151S |
| c.452A > G | c.A452G | Y151C |
| c.454T > A | c.T454A | Y152N |
| c.454T > C | c.T454C | Y152H |
| c.454T > G | c.T454G | Y152D |
| c.455A > C | c.A455C | Y152S |
| c.455A > G | c.A455G | Y152C |
| c.455A > T | c.A455T | Y152F |
| c.457G > A | c.G457A | D153N |
| c.457G > C | c.G457C | D153H |
| c.457G > T | c.G457T | D153Y |
| c.458A > C | c.A458C | D153A |
| c.458A > T | c.A458T | D153V |
| c.465T > A or c.465T > G | c.T465A or c.T465G | D155E |
| c.466G > A | c.G466A | A156T |
| c.466G > T | c.G466T | A156S |
| c.467C > G | c.C467G | A156G |
| c.467C > T | c.C467T | A156V |
| c.469C > A | c.C469A | Q157K |
| c.469C > G | c.C469G | Q157E |
| c.470A > C | c.A470C | Q157P |
| c.470A > T | c.A470T | Q157L |
| c.471G > C or c.471G > T | c.G471C or c.G471T | Q157H |
| c.472A > G | c.A472G | T158A |
| c.472A > T | c.A472T | T158S |
| c.473C > A | c.C473A | T158N |
| c.473C > T | c.C473T | T158I |
| c.475T > A | c.T475A | F159I |
| c.475T > G | c.T475G | F159V |
| c.476T > A | c.T476A | F159Y |
| c.476T > G | c.T476G | F159C |
| c.477T > A | c.T477A | F159L |
| c.478G > A | c.G478A | A160T |
| c.478G > T | c.G478T | A160S |
| c.479C > A | c.C479A | A160D |
| c.479C > G | c.C479G | A160G |
| c.479C > T | c.C479T | A160V |
| c.481G > A | c.G481A | D161N |
| c.481G > C | c.G481C | D161H |
| c.481G > T | c.G481T | D161Y |
| c.482A > T | c.A482T | D161V |
| c.484T > G | c.T484G | W162G |
| c.485G > C | c.G485C | W162S |
| c.490G > A | c.G490A | V164I |
| c.490G > T | c.G490T | V164L |
| c.491T > C | c.T491C | V164A |
| c.493G > A | c.G493A | D165N |
| c.493G > C | c.G493C | D165H |
| c.494A > C | c.A494C | D165A |
| c.494A > G | c.A494G | D165G |
| c.495T > A | c.T495A | D165E |
| c.496_497delinsTC | c.496_497delinsTC | L166S |
| c.496C > A | c.C496A | L166M |
| c.496C > G | c.C496G | L166V |
| c.[496C > G; 497T > G] | c.C496G/T497G | L166G |
| c.497T > A | c.T497A | L166Q |
| c.499C > A | c.C499A | L167I |
| c.499C > G | c.C499G | L167V |
| c.505T > A | c.T505A | F169I |
| c.505T > G | c.T505G | F169V |
| c.506T > A | c.T506A | F169Y |
| c.506T > C | c.T506C | F169S |
| c.506T > G | c.T506G | F169C |
| c.507T > A | c.T507A | F169L |
| c.511G > A | c.G511A | G171S |
| c.512G > C | c.G512C | G171A |
| c.512G > T | c.G512T | G171V |
| c.517T > C | c.T517C | Y173H |
| c.518A > C | c.A518C | Y173S |
| c.518A > G | c.A518G | Y173C |
| c.518A > T | c.A518T | Y173F |
| c.520T > C | c.T520C | C174R |
| c.520T > G | c.T520G | C174G |
| c.523G > C | c.G523C | D175H |
| c.523G > T | c.G523T | D175Y |
| c.524A > G | c.A524G | D175G |
| c.524A > T | c.A524T | D175V |
| c.525C > G or c.525C > A | c.C525G or c.C525A | D175E |
| c.526A > T | c.A526T | S176C |
| c.528T > A | c.T528A | S176R |
| c.529T > A | c.T529A | L177M |
| c.529T > G | c.T529G | L177V |
| c.530T > C | c.T530C | L177S |
| c.530T > G | c.T530G | L177W |
| c.531G > C | c.G531C | L177F |
| c.532G > A | c.G532A | E178K |
| c.532G > C | c.G532C | E178Q |
| c.533A > C | c.A533C | E178A |
| c.533A > G | c.A533G | E178G |
| c.538T > A | c.T538A | L180M |
| c.538T > G | c.T538G | L180V |
| c.539T > C | c.T539C | L180S |
| c.539T > G | c.T539G | L180W |
| c.540G > C or c.540G > T | c.G540C or c.G540T | L180F |
| c.541G > A | c.G541A | A181T |
| c.541G > C | c.G541C | A181P |
| c.542C > T | c.C542T | A181V |
| c.544G > T | c.G544T | D182Y |
| c.545A > C | c.A545C | D182A |
| c.545A > G | c.A545G | D182G |
| c.545A > T | c.A545T | D182V |
| c.546T > A | c.T546A | D182E |
| c.548G > A | c.G548A | G183D |
| c.548G > C | c.G548C | G183A |
| c.550T > A | c.T550A | Y184N |
| c.550T > C | c.T550C | Y184H |
| c.551A > C | c.A551C | Y184S |
| c.551A > G | c.A551G | Y184C |
| c.551A > T | c.A551T | Y184F |
| c.553A > C | c.A553C | K185Q |
| c.553A > G | c.A553G | K185E |
| c.554A > C | c.A554C | K185T |
| c.554A > T | c.A554T | K185M |
| c.555G > C | c.G555C | K185N |
| c.556C > A | c.C556A | H186N |
| c.556C > G | c.C556G | H186D |
| c.556C > T | c.C556T | H186Y |
| c.557A > T | c.A557T | H186L |
| c.558C > G | c.C558G | H186Q |
| c.559_564dup | c.559_564dup | p.M187_S188dup |
| c.559A > T | c.A559T | M187L |
| c.559A > G | c.A559G | M187V |
| c.560T > C | c.T560C | M187T |
| c.561G > T or c.561G > A | c.G561T or c.G561A | M187I |
| or c.561G > C | or c.G561C | |
| c.562T > A | c.T562A | S188T |
| c.562T > C | c.T562C | S188P |
| c.562T > G | c.T562G | S188A |
| c.563C > A | c.C563A | S188Y |
| c.563C > G | c.C563G | S188C |
| c.563C > T | c.C563T | S188F |
| c.565T > G | c.T565G | L189V |
| c.566T > C | c.T566C | L189S |
| c.567G > C or c.567G > T | c.G567C or c.G567T | L189F |
| c.568G > A | c.G568A | A190T |
| c.568G > T | c.G568T | A190S |
| c.569C > A | c.C569A | A190D |
| c.569C > G | c.C569G | A190G |
| c.569C > T | c.C569T | A190V |
| c.571C > A | c.C571A | L191M |
| c.571C > G | c.C571G | L191V |
| c.572T > A | c.T572A | L191Q |
| c.574A > C | c.A574C | N192H |
| c.574A > G | c.A574G | N192D |
| c.575A > C | c.A575C | N192T |
| c.575A > G | c.A575G | N192S |
| c.576T > A | c.T576A | N192K |
| c.577A > G | c.A577G | R193G |
| c.577A > T | c.A577T | R193W |
| c.578G > C | c.G578C | R193T |
| c.578G > T | c.G578T | R193M |
| c.580A > C | c.A580C | T194P |
| c.580A > G | c.A580G | T194A |
| c.580A > T or c.581C > G | c.A580T or c.C581G | T194S |
| c.581C > A | c.C581A | T194N |
| c.581C > T | c.C581T | T194I |
| c.583G > A | c.G583A | G195S |
| c.583G > C | c.G583C | G195R |
| c.583G > T | c.G583T | G195C |
| c.584G > T | c.G584T | G195V |
| c.586A > G | c.A586G | R196G |
| c.587G > A | c.G587A | R196K |
| c.587G > C | c.G587C | R196T |
| c.587G > T | c.G587T | R196I |
| c.589A > G | c.A589G | S197G |
| c.589A > T | c.A589T | S197C |
| c.590G > A | c.G590A | S197N |
| c.590G > C | c.G590C | S197T |
| c.590G > T | c.G590T | S197I |
| c.593T > C | c.T593C | I198T |
| c.593T > G | c.T593G | I198S |
| c.594T > G | c.T594G | I198M |
| c.595G > A | c.G595A | V199M |
| c.595G > C | c.G595C | V199L |
| c.596T > A | c.T596A | V199E |
| c.596T > C | c.T596C | V199A |
| c.596T > G | c.T596G | V199G |
| c.598T > A | c.T598A | Y200N |
| c.599A > C | c.A599C | Y200S |
| c.599A > G | c.A599G | Y200C |
| c.601T > A | c.T601A | S201T |
| c.601T > G | c.T601G | S201A |
| c.602C > A | c.C602A | S201Y |
| c.602C > G | c.C602G | S201C |
| c.602C > T | c.C602T | S201F |
| c.607G > C | c.G607C | E203Q |
| c.608A > C | c.A608C | E203A |
| c.608A > G | c.A608G | E203G |
| c.608A > T | c.A608T | E203V |
| c.609G > C or c.609G > T | c.G609C or c.G609T | E203D |
| c.610T > G | c.T610G | W204G |
| c.611G > C | c.G611C | W204S |
| c.611G > T | c.G611T | W204L |
| c.613C > A | c.C613A | P205T |
| c.613C > T | c.C613T | P205S |
| c.614C > T | c.C614T | P205L |
| c.616C > A | c.C616A | L206I |
| c.616C > G | c.C616G | L206V |
| c.616C > T | c.C616T | L206F |
| c.617T > A | c.T617A | L206H |
| c.617T > G | c.T617G | L206R |
| c.619T > C | c.T619C | Y207H |
| c.620A > C | c.A620C | Y207S |
| c.620A > T | c.A620T | Y207F |
| c.623T > A | c.T623A | M208K |
| c.623T > G | c.T623G | M208R |
| c.625T > A | c.T625A | W209R |
| c.625T > G | c.T625G | W209G |
| c.627G > C | c.G627C | W209C |
| c.628C > A | c.C628A | P210T |
| c.628C > T | c.C628T | P210S |
| c.629C > A | c.C629A | P210H |
| c.629C > T | c.C629T | P210L |
| c.631T > C | c.T631C | F211L |
| c.631T > G | c.T631G | F211V |
| c.632T > A | c.T632A | F211Y |
| c.632T > C | c.T632C | F211S |
| c.632T > G | c.T632G | F211C |
| c.635A > C | c.A635C | Q212P |
| c.636A > T | c.A636T | Q212H |
| c.637A > C | c.A637C | K213Q |
| c.637A > G | c.A637G | K213E |
| c.638A > G | c.A638G | K213R |
| c.638A > T | c.A638T | K213M |
| c.640C > A | c.C640A | P214T |
| c.640C > G | c.C640G | P214A |
| c.640C > T | c.C640T | P214S |
| c.641C > A | c.C641A | P214H |
| c.641C > G | c.C641G | P214R |
| c.641C > T | c.C641T | P214L |
| c.643A > C | c.A643C | N215H |
| c.643A > G | c.A643G | N215D |
| c.643A > T | c.A643T | N215Y |
| c.644A > C | c.A644C | N215T |
| c.644A > G | c.A644G | N215S |
| c.[644A > G; 937G > T] | c.A644G/G937T | N215S/D313Y |
| c.644A > T | c.A644T | N215I |
| c.645T > A | c.T645A | N215K |
| c.646T > A | c.T646A | Y216N |
| c.646T > C | c.T646C | Y216H |
| c.646T > G | c.T646G | Y216D |
| c.647A > C | c.A647C | Y216S |
| c.647A > G | c.A647G | Y216C |
| c.647A > T | c.A647T | Y216F |
| c.649A > C | c.A649C | T217P |
| c.649A > G | c.A649G | T217A |
| c.649A > T | c.A649T | T217S |
| c.650C > A | c.C650A | T217K |
| c.650C > G | c.C650G | T217R |
| c.650C > T | c.C650T | T217I |
| c.652G > A | c.G652A | E218K |
| c.652G > C | c.G652C | E218Q |
| c.653A > C | c.A653C | E218A |
| c.653A > G | c.A653G | E218G |
| c.653A > T | c.A653T | E218V |
| c.654A > T | c.A654T | E218D |
| c.655A > C | c.A655C | I219L |
| c.655A > T | c.A655T | I219F |
| c.656T > A | c.T656A | I219N |
| c.656T > C | c.T656C | I219T |
| c.656T > G | c.T656G | I219S |
| c.657C > G | c.C657G | I219M |
| c.659G > A | c.G659A | R220Q |
| c.659G > C | c.G659C | R220P |
| c.659G > T | c.G659T | R220L |
| c.661C > A | c.C661A | Q221K |
| c.661C > G | c.C661G | Q221E |
| c.662A > C | c.A662C | Q221P |
| c.662A > G | c.A662G | Q221R |
| c.662A > T | c.A662T | Q221L |
| c.663G > C | c.G663C | Q221H |
| c.664T > A | c.T664A | Y222N |
| c.664T > C | c.T664C | Y222H |
| c.664T > G | c.T664G | Y222D |
| c.665A > C | c.A665C | Y222S |
| c.665A > G | c.A665G | Y222C |
| c.670A > C | c.A670C | N224H |
| c.671A > C | c.A671C | N224T |
| c.671A > G | c.A671G | N224S |
| c.673C > G | c.C673G | H225D |
| c.679C > G | c.C679G | R227G |
| c.682A > C | c.A682C | N228H |
| c.682A > G | c.A682G | N228D |
| c.683A > C | c.A683C | N228T |
| c.683A > G | c.A683G | N228S |
| c.683A > T | c.A683T | N228I |
| c.685T > A | c.T685A | F229I |
| c.686T > A | c.T686A | F229Y |
| c.686T > C | c.T686C | F229S |
| c.687T > A or c.687T > G | c.T687A or c.T687G | F229L |
| c.688G > C | c.G688C | A230P |
| c.689C > A | c.C689A | A230D |
| c.689C > G | c.C689G | A230G |
| c.689C > T | c.C689T | A230V |
| c.694A > C | c.A694C | I232L |
| c.694A > G | c.A694G | I232V |
| c.695T > C | c.T695C | I232T |
| c.696T > G | c.T696G | I232M |
| c.698A > C | c.A698C | D233A |
| c.698A > G | c.A698G | D233G |
| c.698A > T | c.A698T | D233V |
| c.699T > A | c.T699A | D233E |
| c.703T > A | c.T703A | S235T |
| c.703T > G | c.T703G | S235A |
| c.710A > T | c.A710T | K237I |
| c.712A > G | c.A712G | S238G |
| c.712A > T | c.A712T | S238C |
| c.713G > A | c.G713A | S238N |
| c.713G > C | c.G713C | S238T |
| c.713G > T | c.G713T | S238I |
| c.715A > T | c.A715T | I239L |
| c.716T > C | c.T716C | I239T |
| c.717A > G | c.A717G | I239M |
| c.718A > G | c.A718G | K240E |
| c.719A > G | c.A719G | K240R |
| c.719A > T | c.A719T | K240M |
| c.720G > C or c.720G > T | c.G720C or c.G720T | K240N |
| c.721A > T | c.A721T | S241C |
| c.722G > C | c.G722C | S241T |
| c.722G > T | c.G722T | S241I |
| c.724A > C | c.A724C | I242L |
| c.724A > G | c.A724G | I242V |
| c.724A > T | c.A724T | I242F |
| c.725T > A | c.T725A | I242N |
| c.725T > C | c.T725C | I242T |
| c.725T > G | c.T725G | I242S |
| c.726C > G | c.C726G | I242M |
| c.727T > A | c.T727A | L243M |
| c.727T > G | c.T727G | L243V |
| c.728T > C | c.T728C | L243S |
| c.728T > G | c.T728G | L243W |
| c.729G > C or c.729G > T | c.G729C or c.G729T | L243F |
| c.730G > A | c.G730A | D244N |
| c.730G > C | c.G730C | D244H |
| c.730G > T | c.G730T | D244Y |
| c.731A > C | c.A731C | D244A |
| c.731A > G | c.A731G | D244G |
| c.731A > T | c.A731T | D244V |
| c.732C > G | c.C732G | D244E |
| c.733T > G | c.T733G | W245G |
| c.735G > C | c.G735C | W245C |
| c.736A > G | c.A736G | T246A |
| c.737C > A | c.C737A | T246K |
| c.737C > G | c.C737G | T246R |
| c.737C > T | c.C737T | T246I |
| c.739T > A | c.T739A | S247T |
| c.739T > G | c.T739G | S247A |
| c.740C > A | c.C740A | S247Y |
| c.740C > G | c.C740G | S247C |
| c.740C > T | c.C740T | S247F |
| c.742T > G | c.T742G | F248V |
| c.743T > A | c.T743A | F248Y |
| c.743T > G | c.T743G | F248C |
| c.744T > A | c.T744A | F248L |
| c.745A > C | c.A745C | N249H |
| c.745A > G | c.A745G | N249D |
| c.745A > T | c.A745T | N249Y |
| c.746A > C | c.A746C | N249T |
| c.746A > G | c.A746G | N249S |
| c.746A > T | c.A746T | N249I |
| c.747C > G or c.747C > A | c.C747G or c.C747A | N249K |
| c.748C > A | c.C748A | Q250K |
| c.748C > G | c.C748G | Q250E |
| c.749A > C | c.A749C | Q250P |
| c.749A > G | c.A749G | Q250R |
| c.749A > T | c.A749T | Q250L |
| c.750G > C | c.G750C | Q250H |
| c.751G > A | c.G751A | E251K |
| c.751G > C | c.G751C | E251Q |
| c.752A > G | c.A752G | E251G |
| c.752A > T | c.A752T | E251V |
| c.754A > G | c.A754G | R252G |
| c.757A > G | c.A757G | I253V |
| c.757A > T | c.A757T | I253F |
| c.758T > A | c.T758A | I253N |
| c.758T > C | c.T758C | I253T |
| c.758T > G | c.T758G | I253S |
| c.760-762delGTT or c.761-763del | c.760_762delGTT or c.761_763del | p.V254del |
| c.760G > T | c.G760T | V254F |
| c.761T > A | c.T761A | V254D |
| c.761T > C | c.T761C | V254A |
| c.761T > G | c.T761G | V254G |
| c.763G > A | c.G763A | D255N |
| c.763G > C | c.G763C | D255H |
| c.763G > T | c.G763T | D255Y |
| c.764A > C | c.A764C | D255A |
| c.764A > T | c.A764T | D255V |
| c.765T > A | c.T765A | D255E |
| c.766G > C | c.G766C | V256L |
| c.767T > A | c.T767A | V256D |
| c.767T > G | c.T767G | V256G |
| c.769G > A | c.G769A | A257T |
| c.769G > C | c.G769C | A257P |
| c.769G > T | c.G769T | A257S |
| c.770C > G | c.C770G | A257G |
| c.770C > T | c.C770T | A257V |
| c.772G > C or c.772G > A | c.G772C or c.G772A | G258R |
| c.773G > A | c.G773A | G258E |
| c.773G > T | c.G773T | G258V |
| c.775C > A | c.C775A | P259T |
| c.775C > G | c.C775G | P259A |
| c.775C > T | c.C775T | P259S |
| c.776C > A | c.C776A | P259Q |
| c.776C > G | c.C776G | P259R |
| c.776C > T | c.C776T | P259L |
| c.778G > T | c.G778T | G260W |
| c.779G > A | c.G779A | G260E |
| c.779G > C | c.G779C | G260A |
| c.781G > A | c.G781A | G261S |
| c.781G > C | c.G781C | G261R |
| c.781G > T | c.G781T | G261C |
| c.782G > C | c.G782C | G261A |
| c.787A > C | c.A787C | N263H |
| c.788A > C | c.A788C | N263T |
| c.788A > G | c.A788G | N263S |
| c.790G > A | c.G790A | D264N |
| c.790G > C | c.G790C | D264H |
| c.790G > T | c.G790T | D264Y |
| c.793C > G | c.C793G | P265A |
| c.794C > A | c.C794A | P265Q |
| c.794C > T | c.C794T | P265L |
| c.799A > G | c.A799G | M267V |
| c.799A > T | c.A799T | M267L |
| c.800T > C | c.T800C | M267T |
| c.802T > A | c.T802A | L268I |
| c.804A > T | c.A804T | L268F |
| c.805G > A | c.G805A | V269M |
| c.805G > C | c.G805C | V269L |
| c.806T > C | c.T806C | V269A |
| c.808A > C | c.A808C | I270L |
| c.808A > G | c.A808G | I270V |
| c.809T > C | c.T809C | I270T |
| c.809T > G | c.T809G | I270S |
| c.810T > G | c.T810G | I270M |
| c.811G > A | c.G811A | G271S |
| c.[811G > A; 937G > T] | c.G811A/G937T | G271S/D313Y |
| c.812G > A | c.G812A | G271D |
| c.812G > C | c.G812C | G271A |
| c.814A > G | c.A814G | N272D |
| c.818T > A | c.T818A | F273Y |
| c.823C > A | c.C823A | L275I |
| c.823C > G | c.C823G | L275V |
| c.827G > A | c.G827A | S276N |
| c.827G > C | c.G827C | S276T |
| c.829T > G | c.T829G | W277G |
| c.830G > T | c.G830T | W277L |
| c.831G > T or c.831G > C | c.G831T or c.G831C | W277C |
| c.832A > T | c.A832T | N278Y |
| c.833A > T | c.A833T | N278I |
| c.835C > G | c.C835G | Q279E |
| c.838C > A | c.C838A | Q280K |
| c.839A > G | c.A839G | Q280R |
| c.839A > T | c.A839T | Q280L |
| c.840A > T or c.840A > C | c.A840T or c.A840C | Q280H |
| c.841G > C | c.G841C | V281L |
| c.842T > A | c.T842A | V281E |
| c.842T > C | c.T842C | V281A |
| c.842T > G | c.T842G | V281G |
| c.844A > G | c.A844G | T282A |
| c.844A > T | c.A844T | T282S |
| c.845C > T | c.C845T | T282I |
| c.847C > G | c.C847G | Q283E |
| c.848A > T | c.A848T | Q283L |
| c.849G > C | c.G849C | Q283H |
| c.850A > G | c.A850G | M284V |
| c.850A > T | c.A850T | M284L |
| c.851T > C | c.T851C | M284T |
| c.852G > C | c.G852C | M284I |
| c.853G > A | c.G853A | A285T |
| c.854C > G | c.C854G | A285G |
| c.854C > T | c.C854T | A285V |
| c.856C > G | c.C856G | L286V |
| c.856C > T | c.C856T | L286F |
| c.857T > A | c.T857A | L286H |
| c.860G > T | c.G860T | W287L |
| c.862G > C | c.G862C | A288P |
| c.862G > T | c.G862T | A288S |
| c.863C > G | c.C863G | A288G |
| c.863C > T | c.C863T | A288V |
| c.865A > C | c.A865C | I289L |
| c.865A > G | c.A865G | I289V |
| c.866T > C | c.T866C | I289T |
| c.866T > G | c.T866G | I289S |
| c.868A > C or c.868A > T | c.A868C or c.A868T | M290L |
| c.868A > G | c.A868G | M290V |
| c.869T > C | c.T869C | M290T |
| c.870G > A or c.870G > C | c.G870A or c.G870C | M290I |
| or c.870G > T | or c.G870T | |
| c.871G > A | c.G871A | A291T |
| c.871G > T | c.G871T | A291S |
| c.872C > G | c.C872G | A291G |
| c.874G > T | c.G874T | A292S |
| c.875C > G | c.C875G | A292G |
| c.877C > A | c.C877A | P293T |
| c.880T > A | c.T880A | L294I |
| c.880T > G | c.T880G | L294V |
| c.881T > C | c.T881C | L294S |
| c.882A > T | c.A882T | L294F |
| c.883T > A | c.T883A | F295I |
| c.883T > G | c.T883G | F295V |
| c.884T > A | c.T884A | F295Y |
| c.884T > C | c.T884C | F295S |
| c.884T > G | c.T884G | F295C |
| c.886A > G | c.A886G | M296V |
| c.886A > T or c.886A > C | c.A886T or c.A886C | M296L |
| c.887T > C | c.T887C | M296T |
| c.888G > A or c.888G > T | c.G888A or c.G888T | M296I |
| or c.888G > C | or c.G888C | |
| c.889T > A | c.T889A | S297T |
| c.892A > G | c.A892G | N298D |
| c.893A > C | c.A893C | N298T |
| c.893A > G | c.A893G | N298S |
| c.893A > T | c.A893T | N298I |
| c.895G > A | c.G895A | D299N |
| c.895G > C | c.G895C | D299H |
| c.897C > G or c.897C > A | c.C897G or c.C897A | D299E |
| c.898C > A | c.C898A | L300I |
| c.898C > G | c.C898G | L300V |
| c.898C > T | c.C898T | L300F |
| c.899T > C | c.T899C | L300P |
| c.901C > G | c.C901G | R301G |
| c.902G > A | c.G902A | R301Q |
| c.902G > C | c.G902C | R301P |
| c.902G > T | c.G902T | R301L |
| c.904C > A | c.C904A | H302N |
| c.904C > G | c.C904G | H302D |
| c.904C > T | c.C904T | H302Y |
| c.905A > T | c.A905T | H302L |
| c.907A > G | c.A907G | I303V |
| c.907A > T | c.A907T | I303F |
| c.908T > A | c.T908A | I303N |
| c.908T > C | c.T908C | I303T |
| c.908T > G | c.T908G | I303S |
| c.911G > A | c.G911A | S304N |
| c.911G > C | c.G911C | S304T |
| c.911G > T | c.G911T | S304I |
| c.916C > G | c.C916G | Q306E |
| c.917A > C | c.A917C | Q306P |
| c.917A > T | c.A917T | Q306L |
| c.919G > A | c.G919A | A307T |
| c.919G > C | c.G919C | A307P |
| c.919G > T | c.G919T | A307S |
| c.920C > A | c.C920A | A307D |
| c.920C > G | c.C920G | A307G |
| c.920C > T | c.C920T | A307V |
| c.922A > C | c.A922C | K308Q |
| c.922A > G | c.A922G | K308E |
| c.923A > G | c.A923G | K308R |
| c.923A > T | c.A923T | K308I |
| c.924A > T or c.924A > C | c.A924T or c.A924C | K308N |
| c.925G > A | c.G925A | A309T |
| c.925G > C | c.G925C | A309P |
| c.926C > A | c.C926A | A309D |
| c.926C > T | c.C926T | A309V |
| c.928C > A | c.C928A | L310I |
| c.928C > G | c.C928G | L310V |
| c.928C > T | c.C928T | L310F |
| c.931C > A | c.C931A | L311I |
| c.931C > G | c.C931G | L311V |
| c.934C > A | c.C934A | Q312K |
| c.934C > G | c.C934G | Q312E |
| c.935A > G | c.A935G | Q312R |
| c.935A > T | c.A935T | Q312L |
| c.936G > T or c.936G > C | c.G936T or c.G936C | Q312H |
| c.937G > T | c.G937T | D313Y |
| c.[937G > T; 1232G > A] | c.G937T/G1232A | D313Y/G411D |
| c.938A > G | c.A938G | D313G |
| c.938A > T | c.A938T | D313V |
| c.939T > A | c.T939A | D313E |
| c.940A > G | c.A940G | K314E |
| c.941A > C | c.A941C | K314T |
| c.941A > T | c.A941T | K314M |
| c.942G > C | c.G942C | K314N |
| c.943G > A | c.G943A | D315N |
| c.943G > C | c.G943C | D315H |
| c.943G > T | c.G943T | D315Y |
| c.944A > C | c.A944C | D315A |
| c.944A > G | c.A944G | D315G |
| c.944A > T | c.A944T | D315V |
| c.946G > A | c.G946A | V316I |
| c.946G > C | c.G946C | V316L |
| c.947T > C | c.T947C | V316A |
| c.947T > G | c.T947G | V316G |
| c.949A > C | c.A949C | I317L |
| c.949A > G | c.A949G | I317V |
| c.950T > C | c.T950C | I317T |
| c.951T > G | c.T951G | I317M |
| c.952G > A | c.G952A | A318T |
| c.952G > C | c.G952C | A318P |
| c.953C > A | c.C953A | A318D |
| c.953C > T | c.C953T | A318V |
| c.955A > T | c.A955T | I319F |
| c.956T > C | c.T956C | I319T |
| c.957C > G | c.C957G | I319M |
| c.958A > C | c.A958C | N320H |
| c.959A > C | c.A959C | N320T |
| c.959A > G | c.A959G | N320S |
| c.959A > T | c.A959T | N320I |
| c.961C > A | c.C961A | Q321K |
| c.962A > G | c.A962G | Q321R |
| c.962A > T | c.A962T | Q321L |
| c.963G > C or c.963G > T | c.G963C or c.G963T | Q321H |
| c.964G > A | c.G964A | D322N |
| c.964G > C | c.G964C | D322H |
| c.965A > C | c.A965C | D322A |
| c.965A > T | c.A965T | D322V |
| c.966C > A or c.966C > G | c.C966A or c.C966G | D322E |
| c.967C > A | c.C967A | P323T |
| c.968C > G | c.C968G | P323R |
| c.970T > G | c.T970G | L324V |
| c.971T > G | c.T971G | L324W |
| c.973G > A | c.G973A | G325S |
| c.973G > C | c.G973C | G325R |
| c.973G > T | c.G973T | G325C |
| c.974G > C | c.G974C | G325A |
| c.974G > T | c.G974T | G325V |
| c.976A > C | c.A976C | K326Q |
| c.976A > G | c.A976G | K326E |
| c.977A > C | c.A977C | K326T |
| c.977A > G | c.A977G | K326R |
| c.977A > T | c.A977T | K326M |
| c.978G > C or c.978G > T | c.G978C or c.G978T | K326N |
| c.979C > G | c.C979G | Q327E |
| c.980A > C | c.A980C | Q327P |
| c.980A > T | c.A980T | Q327L |
| c.981A > T | c.A981T | Q327H |
| c.983G > C | c.G983C | G328A |
| c.985T > A | c.T985A | Y329N |
| c.985T > C | c.T985C | Y329H |
| c.985T > G | c.T985G | Y329D |
| c.986A > G | c.A986G | Y329C |
| c.986A > T | c.A986T | Y329F |
| c.988C > A | c.C988A | Q330K |
| c.988C > G | c.C988G | Q330E |
| c.989A > C | c.A989C | Q330P |
| c.989A > G | c.A989G | Q330R |
| c.990G > C | c.G990C | Q330H |
| c.991C > G | c.C991G | L331V |
| c.992T > A | c.T992A | L331H |
| c.992T > C | c.T992C | L331P |
| c.992T > G | c.T992G | L331R |
| c.994A > G | c.A994G | R332G |
| c.995G > C | c.G995C | R332T |
| c.995G > T | c.G995T | R332I |
| c.996A > T | c.A996T | R332S |
| c.997C > G | c.C997G | Q333E |
| c.998A > C | c.A998C | Q333P |
| c.998A > T | c.A998T | Q333L |
| c.1000G > C | c.G1000C | G334R |
| c.1001G > A | c.G1001A | G334E |
| c.1001G > T | c.G1001T | G334V |
| c.1003G > T | c.G1003T | D335Y |
| c.1004A > C | c.A1004C | D335A |
| c.1004A > G | c.A1004G | D335G |
| c.1004A > T | c.A1004T | D335V |
| c.1005C > G | c.C1005G | D335E |
| c.1006A > G | c.A1006G | N336D |
| c.1006A > T | c.A1006T | N336Y |
| c.1007A > C | c.A1007C | N336T |
| c.1007A > G | c.A1007G | N336S |
| c.1007A > T | c.A1007T | N336I |
| c.1009T > G | c.T1009G | F337V |
| c.1010T > A | c.T1010A | F337Y |
| c.1010T > C | c.T1010C | F337S |
| c.1010T > G | c.T1010G | F337C |
| c.1011T > A | c.T1011A | F337L |
| c.1012G > A | c.G1012A | E338K |
| c.1013A > C | c.A1013C | E338A |
| c.1013A > G | c.A1013G | E338G |
| c.1013A > T | c.A1013T | E338V |
| c.1014A > T | c.A1014T | E338D |
| c.1015G > A | c.G1015A | V339M |
| c.1016T > A | c.T1016A | V339E |
| c.1016T > C | c.T1016C | V339A |
| c.1021G > C | c.G1021C | E341Q |
| c.1022A > C | c.A1022C | E341A |
| c.1027C > A | c.C1027A | P343T |
| c.1027C > G | c.C1027G | P343A |
| c.1027C > T | c.C1027T | P343S |
| c.1028C > T | c.C1028T | P343L |
| c.1030C > G | c.C1030G | L344V |
| c.1030C > T | c.C1030T | L344F |
| c.1031T > G | c.T1031G | L344R |
| c.1033T > C | c.T1033C | S345P |
| c.1036G > T | c.G1036T | G346C |
| c.1037G > A | c.G1037A | G346D |
| c.1037G > C | c.G1037C | G346A |
| c.1037G > T | c.G1037T | G346V |
| c.1039T > A | c.T1039A | L347I |
| c.1043C > A | c.C1043A | A348D |
| c.1046G > C | c.G1046C | W349S |
| c.1046G > T | c.G1046T | W349L |
| c.1047G > C | c.G1047C | W349C |
| c.1048G > A | c.G1048A | A350T |
| c.1048G > T | c.G1048T | A350S |
| c.1049C > G | c.C1049G | A350G |
| c.1049C > T | c.C1049T | A350V |
| c.1052T > A | c.T1052A | V351E |
| c.1052T > C | c.T1052C | V351A |
| c.1054G > A | c.G1054A | A352T |
| c.1054G > T | c.G1054T | A352S |
| c.1055C > G | c.C1055G | A352G |
| c.1055C > T | c.C1055T | A352V |
| c.1057A > T | c.A1057T | M353L |
| c.1058T > A | c.T1058A | M353K |
| c.1058T > C | c.T1058C | M353T |
| c.1061T > A | c.T1061A | I354K |
| c.1061T > G | c.T1061G | I354R |
| c.1063A > C | c.A1063C | N355H |
| c.1063A > G | c.A1063G | N355D |
| c.1063A > T | c.A1063T | N355Y |
| c.1064A > G | c.A1064G | N355S |
| c.1066C > G | c.C1066G | R356G |
| c.1066C > T | c.C1066T | R356W |
| c.1067G > A | c.G1067A | R356Q |
| c.1067G > C | c.G1067C | R356P |
| c.1067G > T | c.G1067T | R356L |
| c.1069C > G | c.C1069G | Q357E |
| c.1072G > C | c.G1072C | E358Q |
| c.1073A > C | c.A1073C | E358A |
| c.1073A > G | c.A1073G | E358G |
| c.1074G > T or c.1074G > C | c.G1074T or c.G1074C | E358D |
| c.1075A > C | c.A1075C | I359L |
| c.1075A > G | c.A1075G | I359V |
| c.1075A > T | c.A1075T | I359F |
| c.1076T > A | c.T1076A | I359N |
| c.1076T > C | c.T1076C | I359T |
| c.1076T > G | c.T1076G | I359S |
| c.1078G > A | c.G1078A | G360S |
| c.1078G > C | c.G1078C | G360R |
| c.1078G > T | c.G1078T | G360C |
| c.1079G > A | c.G1079A | G360D |
| c.1079G > C | c.G1079C | G360A |
| c.1082G > A | c.G1082A | G361E |
| c.1082G > C | c.G1082C | G361A |
| c.1084C > A | c.C1084A | P362T |
| c.1084C > G | c.C1084G | P362A |
| c.1084C > T | c.C1084T | P362S |
| c.1085C > A | c.C1085A | P362H |
| c.1085C > G | c.C1085G | P362R |
| c.1085C > T | c.C1085T | P362L |
| c.1087C > A | c.C1087A | R363S |
| c.1087C > G | c.C1087G | R363G |
| c.1087C > T | c.C1087T | R363C |
| c.1088G > A | c.G1088A | R363H |
| c.1088G > T | c.G1088T | R363L |
| c.1090T > C | c.T1090C | S364P |
| c.1091C > G | c.C1091G | S364C |
| c.1093T > A | c.T1093A | Y365N |
| c.1093T > G | c.T1093G | Y365D |
| c.1094A > C | c.A1094C | Y365S |
| c.1094A > T | c.A1094T | Y365F |
| c.1096A > C | c.A1096C | T366P |
| c.1096A > T | c.A1096T | T366S |
| c.1097C > A | c.C1097A | T366N |
| c.1097C > T | c.C1097T | T366I |
| c.1099A > C | c.A1099C | I367L |
| c.1099A > T | c.A1099T | I367F |
| c.1101C > G | c.C1101G | I367M |
| c.1102G > A | c.G1102A | A368T |
| c.1102G > C | c.G1102C | A368P |
| c.1103C > G | c.C1103G | A368G |
| c.1105G > A | c.G1105A | V369I |
| c.1105G > C | c.G1105C | V369L |
| c.1105G > T | c.G1105T | V369F |
| c.1106T > C | c.T1106C | V369A |
| c.1106T > G | c.T1106G | V369G |
| c.1108G > A | c.G1108A | A370T |
| c.1108G > C | c.G1108C | A370P |
| c.1109C > A | c.C1109A | A370D |
| c.1109C > G | c.C1109G | A370G |
| c.1109C > T | c.C1109T | A370V |
| c.1111T > A | c.T1111A | S371T |
| c.1112C > G | c.C1112G | S371C |
| c.1117G > A | c.G1117A | G373S |
| c.1117G > T | c.G1117T | G373C |
| c.1118G > C | c.G1118C | G373A |
| c.1120A > G | c.A1120G | K374E |
| c.1121A > C | c.A1121C | K374T |
| c.1121A > G | c.A1121G | K374R |
| c.1121A > T | c.A1121T | K374I |
| c.1123G > C | c.G1123C | G375R |
| c.1124G > A | c.G1124A | G375E |
| c.1124G > C | c.G1124C | G375A |
| c.1126G > A | c.G1126A | V376M |
| c.1126G > C | c.G1126C | V376L |
| c.1127T > A | c.T1127A | V376E |
| c.1127T > G | c.T1127G | V376G |
| c.1129G > A | c.G1129A | A377T |
| c.1129G > C | c.G1129C | A377P |
| c.1129G > T | c.G1129T | A377S |
| c.1130C > G | c.C1130G | A377G |
| c.1135A > G | c.A1135G | N379D |
| c.1136A > C | c.A1136C | N379T |
| c.1136A > T | c.A1136T | N379I |
| c.1137T > A | c.T1137A | N379K |
| c.1138C > A | c.C1138A | P380T |
| c.1138C > G | c.C1138G | P380A |
| c.1139C > A | c.C1139A | P380H |
| c.1139C > G | c.C1139G | P380R |
| c.1139C > T | c.C1139T | P380L |
| c.1142C > A | c.C1142A | A381D |
| c.1147T > A | c.T1147A | F383I |
| c.1148T > A | c.T1148A | F383Y |
| c.1148T > G | c.T1148G | F383C |
| c.1150A > T | c.A1150T | I384F |
| c.1151T > C | c.T1151C | I384T |
| c.1152C > G | c.C1152G | I384M |
| c.1153A > G | c.A1153G | T385A |
| c.1154C > T | c.C1154T | T385I |
| c.1156C > A | c.C1156A | Q386K |
| c.1157A > T | c.A1157T | Q386L |
| c.1158G > C | c.G1158C | Q386H |
| c.1159C > A | c.C1159A | L387I |
| c.1159C > T | c.C1159T | L387F |
| c.1160T > A | c.T1160A | L387H |
| c.1160T > G | c.T1160G | L387R |
| c.1162C > A | c.C1162A | L388I |
| c.1162C > G | c.C1162G | L388V |
| c.1162C > T | c.C1162T | L388F |
| c.1163T > A | c.T1163A | L388H |
| c.1163T > G | c.T1163G | L388R |
| c.1168G > A | c.G1168A | V390M |
| c.1171A > C | c.A1171C | K391Q |
| c.1171A > G | c.A1171G | K391E |
| c.1172A > C | c.A1172C | K391T |
| c.1172A > G | c.A1172G | K391R |
| c.1172A > T | c.A1172T | K391I |
| c.1173A > T | c.A1173T | K391N |
| c.1174A > G | c.A1174G | R392G |
| c.1174A > T | c.A1174T | R392W |
| c.1175G > A | c.G1175A | R392K |
| c.1175G > C | c.G1175C | R392T |
| c.1175G > T | c.G1175T | R392M |
| c.1177A > C | c.A1177C | K393Q |
| c.1177A > G | c.A1177G | K393E |
| c.1178A > C | c.A1178C | K393T |
| c.1179G > C | c.G1179C | K393N |
| c.1180C > A | c.C1180A | L394I |
| c.1181T > A | c.T1181A | L394Q |
| c.1181T > C | c.T1181C | L394P |
| c.1181T > G | c.T1181G | L394R |
| c.1183G > C | c.G1183C | G395R |
| c.1184G > A | c.G1184A | G395E |
| c.1184G > C | c.G1184C | G395A |
| c.1186T > A | c.T1186A | F396I |
| c.1186T > G | c.T1186G | F396V |
| c.1187T > G | c.T1187G | F396C |
| c.1188C > G | c.C1188G | F396L |
| c.1189T > A | c.T1189A | Y397N |
| c.1189T > C | c.T1189C | Y397H |
| c.1190A > C | c.A1190C | Y397S |
| c.1190A > G | c.A1190G | Y397C |
| c.1190A > T | c.A1190T | Y397F |
| c.1192G > A | c.G1192A | E398K |
| c.1192G > C | c.G1192C | E398Q |
| c.1193A > G | c.A1193G | E398G |
| c.1195T > A | c.T1195A | W399R |
| c.1195T > G | c.T1195G | W399G |
| c.1198A > C | c.A1198C | T400P |
| c.1198A > G | c.A1198G | T400A |
| c.1198A > T | c.A1198T | T400S |
| c.1199C > A | c.C1199A | T400N |
| c.1199C > T | c.C1199T | T400I |
| c.1201T > A | c.T1201A | S401T |
| c.1201T > G | c.T1201G | S401A |
| c.1202_1203insGACTTC | c.1202_1203insGACTTC | p.T400_S401dup |
| c.1202C > T | c.C1202T | S401L |
| c.1204A > G | c.A1204G | R402G |
| c.1204A > T | c.A1204T | R402W |
| c.1205G > C | c.G1205C | R402T |
| c.1205G > T | c.G1205T | R402M |
| c.1206G > C | c.G1206C | R402S |
| c.1207T > G | c.T1207G | L403V |
| c.1208T > C | c.T1208C | L403S |
| c.1209A > T | c.A1209T | L403F |
| c.1210A > G | c.A1210G | R404G |
| c.1211G > A | c.G1211A | R404K |
| c.1211G > C | c.G1211C | R404T |
| c.1211G > T | c.G1211T | R404I |
| c.1212A > T | c.A1212T | R404S |
| c.1213A > G | c.A1213G | S405G |
| c.1216C > G | c.C1216G | H406D |
| c.1217A > T | c.A1217T | H406L |
| c.1218C > G | c.C1218G | H406Q |
| c.1219A > T | c.A1219T | I407L |
| c.1220T > C | c.T1220C | I407T |
| c.1221A > G | c.A1221G | I407M |
| c.1222A > C | c.A1222C | N408H |
| c.1222A > G | c.A1222G | N408D |
| c.1222A > T | c.A1222T | N408Y |
| c.1223A > C | c.A1223C | N408T |
| c.1225C > A | c.C1225A | P409T |
| c.1225C > G | c.C1225G | P409A |
| c.1225C > T | c.C1225T | P409S |
| c.1226C > T | c.C1226T | P409L |
| c.1228A > G | c.A1228G | T410A |
| c.1228A > T | c.A1228T | T410S |
| c.1229C > T | c.C1229T | T410I |
| c.1231G > A | c.G1231A | G411S |
| c.1231G > T | c.G1231T | G411C |
| c.1232G > A | c.G1232A | G411D |
| c.1232G > C | c.G1232C | G411A |
| c.1232G > T | c.G1232T | G411V |
| c.1234A > C | c.A1234C | T412P |
| c.1234A > G | c.A1234G | T412A |
| c.1234A > T | c.A1234T | T412S |
| c.1235C > A | c.C1235A | T412N |
| c.1235C > T | c.C1235T | T412I |
| c.1237G > A | c.G1237A | V413I |
| c.1237G > T | c.G1237T | V413F |
| c.1238T > G | c.T1238G | V413G |
| c.1240T > G | c.T1240G | L414V |
| c.1242G > C | c.G1242C | L414F |
| c.1243C > A | c.C1243A | L415I |
| c.1244T > A | c.T1244A | L415H |
| c.1246C > G | c.C1246G | Q416E |
| c.1247A > T | c.A1247T | Q416L |
| c.1248G > C | c.G1248C | Q416H |
| c.1249C > A | c.C1249A | L417I |
| c.1252G > A | c.G1252A | E418K |
| c.1252G > C | c.G1252C | E418Q |
| c.1253A > C | c.A1253C | E418A |
| c.1253A > G | c.A1253G | E418G |
| c.1254A > T | c.A1254T | E418D |
| c.1255A > G | c.A1255G | N419D |
| c.1255A > T | c.A1255T | N419Y |
| c.1256A > C | c.A1256C | N419T |
| c.1256A > G | c.A1256G | N419S |
| c.1256A > T | c.A1256T | N419I |
| c.1258A > C | c.A1258C | T420P |
| c.1258A > T | c.A1258T | T420S |
| c.1259C > A | c.C1259A | T420K |
| c.1259C > G | c.C1259G | T420R |
| c.1261A > G | c.A1261G | M421V |
| c.1261A > T | c.A1261T | M421L |
| c.1262T > A | c.T1262A | M421K |
| c.1262T > C | c.T1262C | M421T |
| c.1262T > G | c.T1262G | M421R |
| c.1263G > C | c.G1263C | M421I |
| c.1265A > C | c.A1265C | Q422P |
| c.1267A > T | c.A1267T | M423L |
| c.1268T > A | c.T1268A | M423K |
| c.1268T > C | c.T1268C | M423T |
| c.1269G > C | c.G1269C | M423I |
| c.1271C > T | c.C1271T | S424L |
| c.1275A > C | c.A1275C | L425F |
| c.1279G > A | c.G1279A | D427N |
| c.1286T > G | c.T1286G | L429R |

### Dosing, Formulation and Administration

In one or more embodiments, the Fabry patient is administered migalastat or salt thereof at a frequency of once every other day (also referred to as “QOD”). In various embodiments, the doses described herein pertain to migalastat hydrochloride or an equivalent dose of migalastat or a salt thereof other than the hydrochloride salt. In some embodiments, these doses pertain to the free base of migalastat. In alternate embodiments, these doses pertain to a salt of migalastat. In further embodiments, the salt of migalastat is migalastat hydrochloride. The administration of migalastat or a salt of migalastat is referred to herein as “migalastat therapy”.

Accordingly, in one or more embodiments, the Fabry patient is administered migalastat of salt thereof in a range of from about 15 mg to about 300 mg, from about 15 mg to about 250 mg, from about 15 mg to about 200 mg, from about 15 mg to about 150 mg or from about 15 mg to about 123 mg at a frequency of once every other day, once every three days, once every four days, once every five days, once every six days or once every seven days. In one or more embodiments, the migalastat or salt thereof is administered at a frequency of once every other day (also referred to as “QOD” or “Q48H”), every four days (also referred to as “Q4D” or “Q96H”) or every seven days (also referred to as “Q7D” or “Q168H”). In some embodiments, dosing intervals may include any dosing interval with more than 48 hours between doses. For example, dosing intervals may include dosing every 72, 96, 120, 144, or 168 hours.

In one or more embodiments, the Fabry patient is administered migalastat FBE in a range of from about 15 mg to about 300 mg, from about 15 mg to about 250 mg, from about 15 mg to about 200 mg, from about 15 mg to about 150 mg, from about 15 mg to about 123 mg, from about 15 mg to about 100 mg, from about 15 mg to about 50 mg, from about 50 mg to about 300 mg, from about 50 mg to about 250 mg, from about 50 mg to about 200 mg, from about 50 mg to about 150 mg, from about 50 mg to about 123 mg, from about 50 mg to about 100 mg, from about 100 mg to about 300 mg, from about 100 mg to about 250 mg, from about 100 mg to about 200 mg, from about 100 mg to about 150 mg, from about 100 mg to about 123 mg, from about 150 mg to about 300 mg, from about 150 mg to about 250 mg, from about 150 mg to about 200 mg, from about 200 mg to about 300 mg, from about 200 mg to about 250 mg or from about 250 mg to about 300 mg at a frequency of once every other day, once every three days, once every four days, once every five days, once every six days or once every seven days.

In one or more embodiments, the Fabry patient is administered migalastat FBE of about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 123 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg or about 300 mg at a frequency of once every other day, once every three days, once every four days, once every five days, once every six days or once every seven days.

Again, it is noted that 150 mg of migalastat hydrochloride is equivalent to 123 mg of the free base form of migalastat. Thus, in one or more embodiments, the dose is 150 mg of migalastat hydrochloride or an equivalent dose of migalastat or a salt thereof other than the hydrochloride salt, administered at a frequency of once every other day, once every three days, once every four days, once every five days, once every six days or once every seven days. In further embodiments, the dose is 150 mg of migalastat hydrochloride administered at a frequency of once every other day. In other embodiments, the dose is 123 mg of the migalastat free base administered at a frequency of once every other day.

In one or more embodiments, the Fabry patient is administered migalastat hydrochloride in a range of from about 15 mg to about 300 mg, from about 15 mg to about 250 mg, from about 15 mg to about 200 mg, from about 15 mg to about 150 mg, from about 15 mg to about 123 mg, from about 15 mg to about 100 mg, from about 15 mg to about 50 mg, from about 50 mg to about 300 mg, from about 50 mg to about 250 mg, from about 50 mg to about 200 mg, from about 50 mg to about 150 mg, from about 50 mg to about 123 mg, from about 50 mg to about 100 mg, from about 100 mg to about 300 mg, from about 100 mg to about 250 mg, from about 100 mg to about 200 mg, from about 100 mg to about 150 mg, from about 100 mg to about 123 mg, from about 150 mg to about 300 mg, from about 150 mg to about 250 mg, from about 150 mg to about 200 mg, from about 200 mg to about 300 mg, from about 200 mg to about 250 mg or from about 250 mg to about 300 mg at a frequency of once every other day, once every three days, once every four days, once every five days, once every six days or once every seven days.

In one or more embodiments, the Fabry patient is administered migalastat hydrochloride of about 15 mg, about 20 mg, about 25 mg, about 30 mg, about 35 mg, about 40 mg, about 42 mg, about 45 mg, about 50 mg, about 55 mg, about 57 mg, about 60 mg, about 65 mg, about 67 mg, about 70 mg, about 75 mg, about 77 mg, about 79 mg, about 80 mg, about 85 mg, about 90 mg, about 94 mg, about 95 mg, about 97 mg, about 100 mg, about 105 mg, about 110 mg, about 115 mg, about 120 mg, about 125 mg, about 128 mg, about 130 mg, about 135 mg, about 140 mg, about 144 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg or about 300 mg at a frequency of once every other day, once every three days, once every four days, once every five days, once every six days or once every seven days.

In some embodiments, the patient weighs in a range of from about 10 kg to about ≥50 kg, from about 10 kg to about ≤50 kg, from about 10 kg to about ≤45 kg, from about 10 kg to about ≤40 kg, from about 10 kg to about ≤35 kg, from about 10 kg to about ≤30 kg, from about 10 kg to about ≤25 kg, from about 10 kg to about ≤20 kg, from about 10 kg to about ≤15 kg, from about 15 kg to about ≥50 kg, from about 15 kg to about ≤50 kg, from about 15 kg to about ≤45 kg, from about 15 kg to about ≤40 kg, from about 15 kg to about ≤35 kg, from about 15 kg to about ≤30 kg, from about 15 kg to about ≤25 kg, from about 20 kg to about ≥50 kg, from about 20 kg to about ≤50 kg, from about 20 kg to about ≤45 kg, from about 20 kg to about ≤40 kg, from about 20 kg to about ≤35 kg, from about 20 kg to about ≤30 kg, from about 20 kg to about ≤25 kg, from about 25 kg to about ≥50 kg, from about 25 kg to about ≤50 kg, from about 25 kg to about ≤45 kg, from about 25 kg to about ≤40 kg, from about 25 kg to about ≤35 kg, from about 25 kg to about ≤30 kg, from about 30 kg to about ≥50 kg, from about 30 kg to about ≤50 kg, from about 30 kg to about ≤45 kg, from about 30 kg to about ≤40 kg, from about 30 kg to about ≤35 kg, from about 35 kg to about ≥50 kg, from about 35 kg to about ≤50 kg, from about 35 kg to about ≤45 kg, from about 35 kg to about ≤40 kg, from about 40 kg to about ≥50 kg, from about 40 kg to about ≤50 kg, from about 40 kg to about ≤45 kg, from about 45 kg to about ≥50 kg or from about 45 kg to about ≤50 kg.

Administration of migalastat or salt thereof according to the present invention may be in a formulation suitable for any route of administration, but is preferably administered in an oral dosage form such as a tablet, capsule or solution. For example, the patient is orally administered capsules each containing 25 mg, 40 mg, 50 mg, 60 mg, 75 mg, 80 mg, 100 mg or 150 mg migalastat hydrochloride (i.e. 1-deoxygalactonojirimycin hydrochloride) or an equivalent dose of migalastat or a salt thereof other than the hydrochloride salt. In another example, the patient is orally administered capsules each containing 150 mg migalastat hydrochloride or an equivalent dose of migalastat or a salt thereof other than the hydrochloride salt.

In various embodiments, the doses described herein pertain to migalastat hydrochloride or an equivalent dose of migalastat or a salt thereof other than the hydrochloride salt. In some embodiments, these doses pertain to the free base of migalastat. In alternate embodiments, these doses pertain to a salt of migalastat. In further embodiments, the salt of migalastat is migalastat hydrochloride. The administration of migalastat or a salt of migalastat is referred to herein as “migalastat therapy”.

The administration of migalastat or salt thereof may be for a certain period of time. In one or more embodiments, the migalastat or salt thereof is administered for a duration of at least 28 days, such as at least 30, 60 or 90 days or at least 4, 6, 8, 12, 16, 26 or 52 weeks or at least 1, 2, 3, 4, 5, 6, 7, 8, 9, 10, 11, 12, 16, 20, 24, 30 or 36 months or at least 1, 2, 3, 4 or 5 years. In some embodiments, the migalastat therapy is of at least about 4 weeks. In various embodiments, the migalastat therapy is a long-term migalastat therapy of at least about 2, 3, 4 or 5 years.

In some embodiments, the PC (e.g., migalastat or salt thereof) is administered orally. In one or more embodiments, the PC (e.g., migalastat or salt thereof) is administered by injection. The PC may be accompanied by a pharmaceutically acceptable carrier, which may depend on the method of administration.

In one or more embodiments, the PC (e.g., migalastat or salt thereof) is administered as monotherapy, and can be in a form suitable for any route of administration, including e.g., orally in the form tablets or capsules or liquid, or in sterile aqueous solution for injection. In other embodiments, the PC is provided in a dry lyophilized powder to be added to the formulation of the replacement enzyme during or immediately after reconstitution to prevent enzyme aggregation in vitro prior to administration.

When the PC (e.g., migalastat or salt thereof) is formulated for oral administration, the tablets or capsules can be prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or another suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active chaperone compound.

The pharmaceutical formulations of the PC (e.g., migalastat or salt thereof) suitable for parenteral/injectable use generally include sterile aqueous solutions (where water soluble), or dispersions and sterile powders for the extemporaneous preparation of sterile injectable solutions or dispersion. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be brought about by various antibacterial and antifungal agents, for example, parabens, chlorobutanol, phenol, benzyl alcohol, sorbic acid, and the like. In many cases, it will be reasonable to include isotonic agents, for example, sugars or sodium chloride. Prolonged absorption of the injectable compositions can be brought about by the use in the compositions of agents delaying absorption, for example, aluminum monosterate and gelatin.

Sterile injectable solutions are prepared by incorporating the purified enzyme (if any) and the PC (e.g., migalastat or salt thereof) in the required amount in the appropriate solvent with various of the other ingredients enumerated above, as required, followed by filter or terminal sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

The formulation can contain an excipient. Pharmaceutically acceptable excipients which may be included in the formulation are buffers such as citrate buffer, phosphate buffer, acetate buffer, bicarbonate buffer, amino acids, urea, alcohols, ascorbic acid, and phospholipids; proteins, such as serum albumin, collagen, and gelatin; salts such as EDTA or EGTA, and sodium chloride; liposomes; polyvinylpyrollidone; sugars, such as dextran, mannitol, sorbitol, and glycerol; propylene glycol and polyethylene glycol (e.g., PEG-4000, PEG-6000); glycerol; glycine or other amino acids; and lipids. Buffer systems for use with the formulations include citrate; acetate; bicarbonate; and phosphate buffers. Phosphate buffer is a preferred embodiment.

The route of administration of the chaperone compound may be oral or parenteral, including intravenous, subcutaneous, intra-arterial, intraperitoneal, ophthalmic, intramuscular, buccal, rectal, vaginal, intraorbital, intracerebral, intradermal, intracranial, intraspinal, intraventricular, intrathecal, intracisternal, intracapsular, intrapulmonary, intranasal, transmucosal, transdermal, or via inhalation.

Administration of the above-described parenteral formulations of the chaperone compound may be by periodic injections of a bolus of the preparation, or may be administered by intravenous or intraperitoneal administration from a reservoir which is external (e.g., an i.v. bag) or internal (e.g., a bioerodable implant).

Embodiments relating to pharmaceutical formulations and administration may be combined with any of the other embodiments of the invention, for example embodiments relating to methods of treating patients with Fabry disease, methods of treating ERT-naïve Fabry patients, methods of treating ERT-experienced Fabry patients, methods of reducing the risk of CBV events, methods of reducing the risk of composite clinical outcomes, methods of assessing symptoms or outcomes of a patient or groups of patients, methods of evaluating a treatment therapy, methods of enhancing α-Gal A in a patient diagnosed with or suspected of having Fabry disease, use of a pharmacological chaperone for α-Gal A for the manufacture of a medicament for treating a patient diagnosed with Fabry disease or to a pharmacological chaperone for α-Gal A for use in treating a patient diagnosed with Fabry disease as well as embodiments relating to amenable mutations, the PCs and suitable dosages thereof.

In one or more embodiments, the PC (e.g., migalastat or salt thereof) is administered in combination with ERT. ERT increases the amount of protein by exogenously introducing wild-type or biologically functional enzyme by way of infusion. This therapy has been developed for many genetic disorders, including LSDs such as Fabry disease, as referenced above. After the infusion, the exogenous enzyme is expected to be taken up by tissues through non-specific or receptor-specific mechanism. In general, the uptake efficiency is not high, and the circulation time of the exogenous protein is short. In addition, the exogenous protein is unstable and subject to rapid intracellular degradation as well as having the potential for adverse immunological reactions with subsequent treatments. In one or more embodiments, the chaperone is administered at the same time as replacement enzyme (e.g., replacement α-Gal A). In some embodiments, the chaperone is co-formulated with the replacement enzyme (e.g., replacement α-Gal A).

In one or more embodiments, a patient is switched from ERT to migalastat therapy. In some embodiments, a patient on ERT is identified, the patient's ERT is discontinued, and the patient begins receiving migalastat therapy. The migalastat therapy can be in accordance with any of the methods described herein. In various embodiments, the patient has some degree of renal impairment, such as mild, moderate or severe renal impairment.

### Administration of Migalastat

In some embodiments, migalastat or salt thereof is administered to an adult patient. In some embodiments, age of the adult patient is ≥18 years. In some embodiments, migalastat or salt thereof is administered to an adolescent patient. In some embodiments, age of the adolescent patient is in a range of from 12 years to <18 years, from 13 years to <18 years, from 14 years to <18 years, from 15 years to <18 years, from 16 years to <18 years, from 17 years to <18 years, from 12 years to ≤17 years, from 13 years to ≤17 years, from 14 years to ≤17 years, from 15 years to ≤17 years, from 16 years to ≤17 years, from 12 years to ≤16 years, from 13 years to ≤16 years, from 14 years to ≤16 years, from 15 years to ≤16 years, from 12 years to ≤15 years, from 13 years to ≤15 years, from 14 years to ≤15 years, from 12 years to ≤14 years, from 13 years to ≤14 years, or from 12 years to ≤13 years.

In some embodiments, migalastat or salt thereof is administered to the patient having a weight a range of from <15 kg to ≥45 kg, from 15 kg to <25 kg, from 25 kg to <35 kg, or from 35 kg to <45 kg. In some embodiments, migalastat or salt thereof is administered to the patient having a weight <15 kg. In some embodiments, migalastat or salt thereof is administered to the patient having a weight ≥45 kg.

In some embodiments, about 25 mg of migalastat or salt thereof is administered to the patient having a weight of <15 kg. In some embodiments, about 50 mg of migalastat or salt thereof is administered to the patient having a weight in a range of from 15 kg to <25 kg. In some embodiments, about 75 mg of migalastat or salt thereof is administered to the patient having a weight in a range of from 25 kg to <35 kg. In some embodiments, about 75 mg of migalastat or salt thereof is administered to the patient having a weight in a range of from 35 kg to <50 kg.

In some embodiments, the migalastat or salt thereof is administered at a first frequency for a first time period, and then administered at a second frequency for a second time period. The first frequency is greater (i.e., more frequent) than the second frequency. The first frequency and the second frequency may be any dosing interval disclosed herein. In some embodiments, the first frequency is every other day and the second frequency is every three days, every four days, every five days, every six days or every seven days. In some embodiments, the first frequency is every four days and the second frequency is every five days, every six days, or every seven days.

In some embodiments, the migalastat or salt thereof is administered at a first frequency for a first time period, then administered at a second frequency for a second time period, and then administered at a third frequency for a third time period. The first frequency is greater (i.e., more frequent) than the second frequency, and the second frequency is greater than the third frequency. For example, in some embodiments, the migalastat or salt thereof is administered at a first frequency of once every other day for a first time period, then the migalastat or salt thereof is administered at a second frequency of once every four days for a second time period, and then the migalastat or salt thereof is administered at a third frequency of once every seven days for a third time period.

### Monitoring Lyso-Gb3 and Migalastat Levels

Lyso-Gb3 (globotriaosylsphingosine) can be monitored to determine whether substrate is being cleared from the body of a Fabry patient. Higher levels of lyso-Gb3 correlate with higher levels of substrate. If a patient is being successfully treated, then lyso-Gb3 levels are expected to drop. One dosing regimen for Fabry disease is administering to the patient about 20 mg to about 300 mg FBE of migalastat or salt thereof at a frequency of once every other day.

In some embodiments, the method further comprises measuring migalastat levels. In one or more embodiments, migalastat concentration (e.g., ng/ml) is measured. In some embodiments, the total area under the curve (AUC_{0-∞}) is measured. In one or more embodiments, the lowest concentration the migalastat reaches before the next dose (C_{trough}) is measured.

Migalastat levels can be measured via methods known in the art. For example, if measuring migalastat from tissue samples, tissue aliquots may be homogenized (7 μL water per 1 mg tissue) using a homogenizer (e.g., FastPrep-24 from MP Biomedical, Irvine, CA). Microcentrifuge tubes containing 100 μl of the tissue homogenate or 50 μl of plasma may then be spiked with 500 ng/ml 13C d2-AT1001 HCl internal standard (manufactured by MDS Pharma Services). A 600 μl volume of 5 mM HCl in 95/5 MeOH:H₂O can then be added and the tubes vortexed for 2 minutes, followed by centrifugation at 21000×g for 10 minutes at room temperature. The supernatants may then be collected into a clean, 96-well plate, diluted with 5 mM HCl in dH₂O and applied to a 96-well solid phase extraction (SPE) plate (Waters Corp., Milford MA). After several wash steps and elution into a clean, 96-well plate, the extracts may be dried down under N₂ and reconstituted with mobile phase A. Migalastat levels can then be determined by liquid chromatography-tandem mass spectroscopy (LC-MS/MS) (e.g., LC: Shimadzu; MS/MS: ABSciex API 5500 MS/MS). The liquid chromatography can be conducted using an ACN:water:formate binary mobile phase system (mobile phase A: 5 mM ammonium formate, 0.5% formic acid in 95:5 ACN:water; mobile phase B: 5 mM ammonium formate, 0.5% formic acid in 5:47.5:47.5 ACN:MeOH:water) with a flow rate of 0.7 mL/minute on an Halo HILIC column (150×4.6 mm, 2.7 μm) (Advanced Materials Technology, Inc.). MS/MS analysis may be carried out under APCi positive ion mode. The same procedure may be followed for migalastat determination in plasma except without homogenization. The following precursor ion->product ion transitions may be monitored: mass/charge (m/z) 164.1→m/z 80.1 for migalastat and m/z 167.1→m/z 83.1 for the internal standard. A 12-point calibration curve and quality control samples may be prepared. The ratio of the area under the curve for migalastat to that of the internal standard is then determined and final concentrations of migalastat in each sample calculated using the linear least squares fit equation applied to the calibration curve. To derive approximate molar concentrations, one gram of tissue may be estimated as one mL of volume.

In some embodiments, samples may be taken at 0, 1, 2, 3, 4, 6, 8, 12, 24, 48, 72, 96, 120, 144 and/or 168 hours after administration. In some embodiments, the migalastat concentration 48 hours after administration is measured. In some embodiments, the administration of the second time period is begun after more than about 5, 10, 15, 20, 25, 40, 50, 60, 70, 80, 90, 100, 125, 150, 175 or 200 ng/mL of migalastat is measured 48 hours after administration of the migalastat during the first time period is measured.

In some embodiments, Lyso-Gb3 can be measured via methods known in the art using validated assays. As with migalastat, lyso-Gb3 levels may be determined using liquid chromatography—tandem mass spectroscopy (LC-MS/MS) (e.g., LC: Shimadzu; MS/MS: ABSciex API 5500 MS/MS). For example, one process of measuring plasma lyso-Gb3 is described in Hamler, Rick, et al. “Accurate quantitation of plasma globotriaosylsphingosine (lyso-Gb3) in normal individuals and Fabry disease patients by liquid chromatography-tandem mass spectrometry (LC-MS/MS).” Molecular Genetics and Metabolism, Volume 114.2 (2015):S51. In one or more embodiments, lyso-Gb3 is measured in samples from a patient's urine.

Dose Adjustment

In some embodiments, the dosing frequency of migalastat or salt thereof is adjusted in response to a change in the patient's eGFR. In exemplary embodiments, when the patient's eGFR is reduced below 60 mL/min/1.73 m², below 45 mL/min/1.73 m², below 30 mL/min/1.73 m² or below 15 mL/min/1.73 m², the dosing frequency can be reduced. In some embodiments, the patient is not administered migalastat or salt thereof, when the patient's eGFR is reduced below 60 mL/min/1.73 m², below 45 mL/min/1.73 m², below 30 mL/min/1.73 m² or below 15 mL/min/1.73 m².

Migalastat concentration can be measured from plasma samples at various times to monitor clearance from the body. A clinically relevant increase in C_{trough} suggests significant accumulation of plasma migalastat concentration. If the migalastat is not cleared from the body enough prior to the next dose administration, then the levels of migalastat can build up, possibly leading to an inhibitory effect. Thus, in one or more embodiments, a change in the dosing frequency occurs after a 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0-fold increase in C_{trough} compared to normal renal function C_{trough}.

In one or more embodiments, a change in the dosing frequency occurs after a 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0-fold increase in AUC_{0-∞} compared to normal renal function AUC_{0-∞}.

In some embodiments, the method further comprises measuring lyso-Gb3 in one or more plasma samples from the patient. A first baseline lyso-Gb3 level may be determined during the first time period. As used herein, “baseline lyso-Gb3 level” refers to the lowest plasma lyso-Gb3 value measured during a given time period or dosing regimen. Thus, if the lyso-Gb3 levels go up significantly from the baseline lyso-Gb3 levels, this may indicate kidney disease progression and/or improper clearance of migalastat. Thus, in further embodiments, the administration of the second time period is begun after an increase (e.g., of at least about 20, 25, 30, 33, 35, 40, 45 or 50% and/or 1, 1.25, 1.5, 1.75, 2, 2.25, 2.5 or 3 nM) above the first baseline lyso-Gb3 level is measured. A 33% and/or 2 nM increase from baseline in plasma lyso-Gb3 has been deemed clinically relevant based upon Phase 3 data in Fabry patients signaling either inhibition-induced migalastat exposure from decline in renal function and/or progression of disease condition. Lyso-Gb3 levels may be measured at varying frequencies (e.g., about once every 2, 3, 4 or 5 months). It is thought that it takes about 3 months for a baseline lyso-Gb3 level to be established once a dosing regimen has been started.

In some embodiments, the administration of the second time period may begin after an increase above the first baseline lyso-Gb3 level is at least about 30, or 33% and/or 2 nM and/or more than about 50 ng/ml of migalastat is measured 48 hours after administration of the migalastat during the first time period is measured. In some embodiments, the administration of the second time period may begin after an increase above the first baseline lyso-Gb3 level is at least about 30, or 33% and/or 2 nM and/or more than about 50 ng/ml of migalastat is measured 48 hours after administration of the migalastat during the first time period is measured, or there is a greater than 1.5-fold increase in AUC_{0-∞} and/or C_{trough} compared to normal renal function during the first time period.

### EXAMPLES

### Example 1: Dosing Regimens for the Treatment of ERT-Experienced and ERT-Naïve Fabry Patients Using Migalastat Hydrochloride

This example describes Phase 2 and Phase 3 studies of migalastat therapy in ERT-experienced and ERT-naïve Fabry patients.

### Study Designs

These analyses included data from 4 Phase 2 and 4 Phase 3 clinical trials with the data cutoff of Feb. 10, 2017 as shown in Figure X1 below.

FAB-CL-202 (NCT00283959), FAB-CL-203 (NCT00283933), and FAB-CL-204 (NCT00304512) were phase 2, open-label, noncomparative studies that evaluated the safety, tolerability, pharmacokinetics (PK), and pharmacodynamics (PD) of migalastat (dose range: 50-250 mg) in patients with Fabry disease.

FAB-CL-205 (NCT0052607) was a phase 2, long-term, open-label extension (OLE) study for patients completing phase 2 clinical trials, including FAB-CL-202, FAB-CL-203, and FAB-CL-204. The study included a period with migalastat 150 mg every other day (QOD), then a dose-escalation period, followed by 150 mg QOD.

FACETS (AT1001-011, NCT00925301) was a phase 3, placebo-controlled study designed to evaluate the efficacy, safety, and PD of 6 months of migalastat 150 mg QOD versus placebo, followed by an 18-month open label extension (OLE) of migalastat in ERT-naive patients with Fabry disease and migalastat-amenable GLA variants.

ATTRACT (AT1001-012, NCT01218659) was a phase 3, open-label, active-controlled study to compare the efficacy and safety of 18 months of migalastat 150 mg QOD versus ERT, followed by a 12-month OLE of migalastat, in ERT-treated patients with migalastat-amenable GLA variants.

AT1001-041 (NCT01458119) was a long-term OLE study evaluating the long-term safety and efficacy of migalastat in patients completing FAB-CL-205, AT1001-011, or AT1001-012

AT1001-042 (NCT02194985) is an ongoing, long-term OLE study evaluating the long-term safety and efficacy of migalastat in patients who participated in AT1001-012 or AT1001-041.

### Analyses

The analysis evaluates CBV events reported as treatment-emergent adverse events (TEAEs) during migalastat 150 mg QOD treatment in patients with amenable mutations in phase 2 and phase 3 clinical trials.

CBV events were identified by searching medical history and TEAE listings with stroke-related terms, including brain stem ischemia, cerebral infarction, cerebral hemorrhage, cerebral ischemia, cerebrovascular accident, embolic stroke, and TIA.

Only amenable patients who received at least 1 dose of migalastat 150 mg QOD were included in this analysis.

Amenability was based on results from a good laboratory practice (GLP)-validated, in vitro migalastat amenability assay.

Clinical Studies Included in the Analysis

### Results

### Migalastat 150 mg QOD Total Exposure

The total mean (SD) duration of exposure to migalastat 150 mg QOD was 4.0 (2.0) years (N=114).

The duration of exposure to migalastat 150 mg QOD ranged from 0.1 to 8.3 years, with a median of 4.4 years.

### Demographics and Baseline Characteristics

The mean (SD) age of all amenable patients receiving at least 1 dose of migalastat 150 mg QOD was 46.2 (13.1) years (range: 16 to 72 years) (Table 2). The majority were white, and 57.0% were female. The mean (SD) time since diagnosis of Fabry disease was 9.8 (10.1) years (range: 1 to 44 years).

TABLE 2
Demographics and Baseline Characteristics: All Amenable
Patients Receiving Migalastat 150 mg QOD

| | FAB-CL 202, | | | |
|---|---|---|---|---|
| | 203, 204, | | | |
| | and 205 | FACETS | ATTRACT | Total |
| Parameter | (n = 17) | (n = 48) | (n = 49) | (N = 114) |

Age, year

| | | | | | | | | |
|---|---|---|---|---|---|---|---|---|
| Mean (SD) | 43.1 | (13.4) | 43.4 | (11.2) | 50.0 | (14.0) | 46.2 | (13.1) |

| | | | | | | | |
|---|---|---|---|---|---|---|---|
| Median (range) | 42.0 | (18, 65) | 46.0 | (16, 68) | 54.0 | (18, 72) | 46.5 |

Age group, n (%)

| <18 years old | 0 | | 1 | (2.1) | 0 | | 1 | (0.9) |
|---|---|---|---|---|---|---|---|---|
| 18 to <65 years old | 15 | (88.2) | 46 | (95.8) | 42 | (85.7) | 103 | (90.4) |
| ≥65 years old | 2 | (11.8) | 1 | (2.1) | 7 | (14.3) | 10 | (8.8) |

Sex, n (%)

| | | | | | | | | |
|---|---|---|---|---|---|---|---|---|
| Female | 5 | (29.4) | 30 | (62.5) | 30 | (61.2) | 65 | (57.0) |
| Male | 12 | (70.6) | 18 | (37.5) | 19 | (38.8) | 49 | (43.0) |

Years diagnosed with Fabry disease

| | | | | | | | | |
|---|---|---|---|---|---|---|---|---|
| Mean (SD) | 7.2 | (7.5) | 7.6 | (7.8)^{a} | 12.9 | (12.1) | 9.8 | (10.1) |
| Median (range) | 5.0 | (2, 34) | 5.0 | (1, 25) | 7.0 | (3, 44) | 6.0 | (1, 44) |

ACEI=angiotensin-converting enzyme inhibitor; ARB-angiotensin receptor block; RI=renin inhibitor; SD=standard deviation.
^{a}Fabry disease diagnosis date was not recorded for 1 patient in FACETS.

### Medical History of CBV Events

Sixteen of 114 patients (14%) had experienced CBV events prior to migalastat treatment (Table 3). One patient from Study AT1001-012 had reported 2 CBV events in medical history.

In 5/16 patients, the CBV events were considered a current condition at study entry as reported in medical history. One patient from AT1001-011 had ongoing cerebral ischemia; another had ongoing brain stem infarction. Two patients from AT1001-012 had ongoing TIA, one had an ongoing cerebrovascular accident, specifically left middle cerebral artery stroke

The mean (SD) age at the time of first CBV event was 43.6 (14.4) years.

TABLE 3
Medical History of CBV Events.

| | FAB-CL 202, | | | |
|---|---|---|---|---|
| | 203, 204, | | | |
| | and 205 | FACETS | ATTRACT | Total |
| | (n = 17) | (n = 48) | (n = 49) | (N = 114) |
| Brain stem | 0 | 0 | 0 | 0 |
| ischemia | | | | |
| Cerebral | 0 | 1 (2%) | 0 | 1 (1%) |
| infarction | | | | |
| Cerebral | 0 | 0 | 0 | 0 |
| hemorrhage | | | | |
| Cerebral | 0 | 1 (2%) | 1 (2%) | 2 (2%) |
| ischemia | | | | |
| Cerebrovascular | 0 | 2 (4%) | 3 (6%) | 5 (4%) |
| accident | | | | |
| Embolic stroke | 0 | 0 | 0 | 0 |
| Transient | 2 (12%) | 2 (4%) | 5 (10%) | 9 (8%) |
| ischemic attack | | | | |
| Any CBV event | 2 (12%) | 6 (12%) | 8 (16%) | 16 (14%) |
| history^{a} | | | | |

^{a}The last row shows number of unique patients with CBV events. The 1 patient with >1 CBV event was only counted once.

### Occurrence of CBV Events During Migalastat 150 mg QOD Treatment

Eleven CBV events were reported during treatment with migalastat 150 mg QOD in 8 patients (7%) (Table 4). Seven CBV events were categorized as serious adverse events (SAE); however, most (82%) events were mild or moderate in severity (Table 5). Two CBV events led to treatment discontinuation (Table 5). None of the 11 CBV events were considered related to treatment

10 Six out of the 8 patients had experienced CBV prior to receiving migalastat treatment; thus only 2/114 (2%) patients had a first CBV event while on migalastat (Table 5). The mean (SD) age of patients at first event during migalastat treatment was 50.6 (14.6) years (Table 5). The mean (SD) time on migalastat 150 mg QOD at first event onset was 1.1 (1.1) years.

Among the 16 patients with pre-migalastat CBV event, 10 (63%) did not experience new CBV event during migalastat treatment.

TABLE 4
CBV Events During Treatment With
Migalastat 150 mg QOD by Trial

| | FAB-CL 202, | | | |
|---|---|---|---|---|
| | 203, 204, | | | |
| | and 205 | FACETS | ATTRACT | Total |
| | (n = 17) | (n = 48) | (n = 49) | (N = 114) |
| Brain stem | 0 | 1 (2%) | 0 | 1 (1%) |
| ischemia | | | | |
| Cerebral | 1 (6%) | 0 | 0 | 1 (1%) |
| infarction | | | | |
| Cerebral | 0 | 1 (2%) | 0 | 1 (1%) |
| hemorrhage | | | | |
| Cerebral | 1 (6%) | 0 | 0 | 1 (1%) |
| ischemia | | | | |
| Cerebrovascular | 1 (6%) | 0 | 0 | 1 (1%) |
| accident | | | | |
| Embolic stroke | 0 | 0 | 0 | 0 |
| Transient | 1 (6%) | 1 (2%) | 2 (4%) | 4 (4%) |
| ischemic attack | | | | |
| Any CBV event^{a} | 3 (18%) | 3 (6%) | 2 (4%) | 8 (7%) |

^{a}The last row shows number of unique patients with CBV events. Patients with >1 CBV event were only counted once.

| | | | | | | Time on | | | | | |
|---|---|---|---|---|---|---|---|---|---|---|---|
| | | | | | | migalastat | | | | | |
| | | | | | Age at | 150 mg | | | | | |
| | | | | Event | event | QOD at | | | | | History |
| Patient | | | | duration | onset | onset | | | | Treatment | of CBV |
| No. | Sex | Mutation | CBV Event | (day) | (year) | (day) | Serious | Severity | Resolved | discontinued | event |
| 1 | F | M284T | TIA (right | 3 | 38 | 589 | Yes | Mild | Yes | No | CBV |
| | | | eye) | | | | | | | | accident |
| 2 | M | I253T | Cerebral | Ongoing^{b} | 63 | 289 | Yes | Severe | No | Yes | None |
| | | | hemorrhage | | | | | | | | |
| 3 | M | I253T | Brain stem | 47 | 66 | 1413 | Yes | Moderate | Yes with | No | Cerebral |
| | | | ischemia | | | | | | sequela | | ischemia |
| | | | (left pontine | | | | | | | | |
| | | | region) | | | | | | | | |
| 4 | M | G35R | TIA | 7 | 59 | 24 | Yes | Moderate | Yes | No | TIA |
| 5 | F | L32P | TIA | 1 | 56 | 559 | No | Mild | Yes | No | TIA |
| | | | TIA | 1 | 56 | 624 | No | Moderate | Yes | No | |
| 6 | M | P259R | CBV | 220 | 21 | 90 | Yes | Severe | Yes with | Yes | None |
| | | | accident | | | | | | sequela | | |
| 7 | F | R112H | TIA | 3 | 39 | 21 | Yes | Moderate | Yes | No | TIA |
| | | | TIA | 2 | 41 | 666 | Yes | Moderate | Yes | No | |
| 8 | F | P205T | Cerebral | Ongoing^{c} | 60 | 371 | No | Moderate | No | No | TIA |
| | | | infarction | | | | | | | | |
| | | | (right | | | | | | | | |
| | | | middle | | | | | | | | |
| | | | artery) | | | | | | | | |
| | | | Abnormal | Ongoing^{c} | 60 | 182 | No | Moderate | No | No | |
| | | | MRI finding | | | | | | | | |
| | | | (old | | | | | | | | |
| | | | ischemic | | | | | | | | |
| | | | change of | | | | | | | | |
| | | | right frontal | | | | | | | | |
| | | | lobe) | | | | | | | | |

TABLE 5
CBV Events During Treatment With Migalastat 150 mg QOD by Patient.

| | Baseline Parameters^{a} | | | | | |
|---|---|---|---|---|---|---|
| | Time since | Blood | | | | |
| Patient | diagnosis | Pressure | Urine Protein | eGFR_{CKD-EPI} | LVMi | ACEI/ARB/RI |
| No. | (year) | (mm Hg) | (mg/24 hr) | (mL/min/1.73 m²) | (g/m²) | use |
| 1 | 14.5 | 120/80 | 399 | 81.8 | 88.7 | No |
| 2 | 0.3 | NA | 1900 | 53.8 | 142.8 | No |
| 3 | 4.6 | 120/80 | 331 | 86.8 | 176.2 | Yes |
| 4 | 10 | 110/67 | 182 | 84.9 | 154.9 | Yes |
| 5 | 2.2 | 112/64 | 231 | 73.2 | 68.6 | Yes |
| 6 | 8.3 | 110/70 | 66 | 137.4 | NA | No |
| 7 | 5.8 | 113/73 | 163 | 120.0 | NA | No |
| 8 | 1.9 | 129/69 | NA | NA^{d} | NA | No |

LVMi = left ventricular mass index; MRI = magnetic resonance imaging; NA = not applicable; TIA = transient ischemic attack.
^{a}Baseline was the start of migalastat 150 mg QOD.
^{b}Ongoing at the time of discontinuation in FACETS.
^{c}Ongoing at the end of FAB-CL-204.
^{d}This patient had an eGFR_{MDRD} of 76.4 mL/min/1.73 m².

As can be seen from the tables above, overall incidence of CBV events was low during migalastat treatment. During an average of 4 years of migalastat, 8/114 (7%) patients had experienced CBV events, predominantly occurring in patients with a history of CBV events.

### Example 2: Simulation of PK/PD Parameters in Adolescents PK/PD Modelling

A population pharmacokinetics (popPK) model previously developed from healthy adult volunteers and adult patients with Fabry disease after oral migalastat administration. After pooling plasma concentration-time data from Phase I, II, and III studies of AT1001 administered orally in adults using a range of doses from 25 mg to 675 mg and regimens under fasting conditions. The conclusions made based on AT1001 study includes:
A two-compartment population pharmacokinetic model with linear time-dependent absorption characterizes the pharmacokinetics of migalastat in plasma after oral administration.
Renal function is the most important determinant of variability in migalastat exposure, with an average 3-fold range occurring for eGFR values between 30 and 120 mL/min/1.73 m².
Subject weight is the second-largest determinant of variability in migalastat exposure, with an average <2-fold difference for body weights between 50 and 170 kg.
The dose rationale for adults (123 mg every other day (QOD)) was supported by the evaluation of several dose levels and regimens in the 4 Phase II studies (50, 150, and 250 mg QOD; 50 mg once daily; 25, 100, and 250 mg twice daily; and 250 and 500 mg ×3 days and off 4 days).
The present population PK model was considered appropriate for adults; however, it does not have an allometric component with standard exponents (e.g. 0.75 for CLT/F), making pediatric predictions less feasible. Thus, the adult population PK model requires some adjustments to allow extrapolation of migalastat PK to the pediatric age sub-groups of 2 to <6, 6 to <12 and 12 to <18 years.
The population PK model of migalastat showed that subject weight (WT) and/or renal function (estimated glomerular filtration rate, eGFR) at baseline significantly impacted the apparent oral plasma clearance (CLT/F) and apparent oral volume of distribution of the central compartment (V₂/F). In contrast, other covariates such as sex, age, drug formulation (solution or suspension vs 25 mg capsule vs 150 mg capsule) were not statistically/clinically significant. Since renal function gradually increases from birth and reaches adult levels by the second year of life (Rubin 1949), there are no expected age-dependent changes in eGFR in the pediatric population 2 years and older than adults. Additionally, pediatric patients with Fabry disease usually have a normal renal function or may experience renal hyperfiltration (Hopkin 2008); therefore, weight-based dosing regimens, assuming that pediatrics have a normal renal function, were planned for the simulations in pediatric Fabry patients.

NONMEM program was used to develop the population PK model of migalastat in adults using first-order conditional estimation with interaction (FOCE-I). Simulations were conducted using NONMEM to obtain plasma concentration-time; all graphical analyses were performed using R; noncompartmental analysis and pharmacokinetic parameters summaries were conducted using Phoenix WinNonlin. Bootstrapping and visual predictive checks (VPC)s were conducted using Perl-speaks-NONMEM (PsN) R packages of popED and mrgsolve were used in the optimal sampling strategy.

The population PK model was optimized by one or more of re-examine absorption models, adding allometric scaling components to CLT/F and Q/F with an allometric exponent equal to 0.75 and to V₂/F and V₃/F with an allometric exponent equal to 1.0, and evaluating whether the allometric exponent should be on total CLT/F or on the non-renal clearance only.

The original linear time-dependent absorption model was chosen among the different absorption models because the conditional weighted residual (CWRES) over time plots were substantially improved, with much less bias and fluctuation throughout the profile. Because the time varying Kₐ model allows Kₐ to continuously increase, an upper limit of time-dependent absorption coefficient Kₐ was set up at 24 hours post-dose to provide reasonable Kₐvalues in simulation/predictions; this was considered to be a minimal change to the original model as the drug is considered to be fairly fully absorbed within 7-10 hours, regardless of the model chosen.

The overall purpose of the model development was to come up with a model for pediatric extrapolation. The theoretical power model indices of 0.75 (for CL and Q), and 1 (for V2 and V3) were applied and evaluated. The diagnostic plots suggested that allometric scaling was only appropriate for those <70 kg.

The final equations for CLT/F, Q/F, V2/F and V3/F were presented as follows:

$$WTCO = WT / 70 when WT \leq 70 ; WTCO = 1 when WT > 70 , \\ where WTCO was the allometric weight coefficient with allometric scaling for subjects with weight \leq 70 kg .$$

$${CL}_{T} / F = tvCL * {( RF )}^{CLEGFR} * {WTCO}^{0.75} * {( 1 + CLHVT )}^{1 - FBRY} * exp ( ETA of IIV on CL / F )$$

$${V}_{2} / F = {tvV}_{2} * {WTCO}^{1} * {( 1 + {V}_{2} HVT )}^{1 - FBRY} * exp ( ETA of IIV on {V}_{2} / F )$$

$$Q / F = TVQ * {WTCO}^{0.75} and {V}_{3} / F = TVV 3 * {WTCO}^{1} , \\ where TVQ and TVV 3 were the typical value of Q / F or {V}_{3} / F , respectively .$$

Considering that renal function is comparable between pediatric patients 2 years and up and adults, the model was modified to apply the allometric exponent to only the non-renal clearance component. The model that successfully converged suggested only a very small portion of CLT/F was accounted for by non-renal clearance; therefore, the allometric scaling applied to this very small non-renal clearance did not really impact the overall CL_{T}/F. The diagnostic plots also suggested that applying the allometric exponent to overall CL_{T}/F for subjects <70 kg was better than applying it to the non-renal clearance. Moreover, pediatric CL_{T}/F values extrapolated from the non-renal model were higher than the overall CL_{T}/F approach, resulting in higher pediatric doses for achieving equivalent exposures with adults which was a less conservative approach. Therefore, the overall CL_{T}/F scaling approach is more conservative and was chosen for the final model, which is shown in Table 6.

TABLE 6
Parameter estimates from the Final Optimized popPK
model of migalastat (with and without bootstrap).

| | NONMEM | | Bootstrap | |
|---|---|---|---|---|
| | Estimate (% RSE); | IIV | Estimate (% RSE); | IIV |
| Parameter | [95% CI] | (% CV) | [95% CI] | (% CV) |
| Typical EGFR-related estimate | 18.6 (16.3%); | 28.8% | 18.5 (16.0%); | 28.5% |
| for those with Fabry disease, with | [12.6, 24.6] | | [14.0, 25.4] | |
| EGFR = 90 mL/min/1.73 m², and | | | | |
| with body weight ≥ 70 kg | | | | |
| Typical EGFR-related estimate | 20.9 (17.4%); | | 20.6 (17.3%); | |
| for those with Fabry disease, with | [13.8, 28.0] | | [15.4, 28.8] | |
| EGFR > 120 mL/min/1.73 m², | | | | |
| and with body weight ≥ 70 kg | | | | |
| EGFR-related exponential index | 0.922 (5.64%); | | 0.925 (5.25%); | |
| on CL/F | [0.820, 1.02] | | [0.832, 1.02] | |
| Typical total CL/F (L/h) for those | 14.8 | | 14.9 | |
| with Fabry disease, with EGFR = | | | | |
| 90 mL/min/1.73 m², and with | | | | |
| body weight ≥ 70 kg ^{a} | | | | |
| Typical total CL/F (L/h) for those | 16.5 | | 16.4 | |
| with Fabry disease, with EGFR = | | | | |
| 120 mL/min/1.73 m², and with | | | | |
| body weight ≥ 70 kg ^{b} | | | | |
| Typical V₂/F (L) for those with | 70.1 (5.29%); | 34.5% | 69.7 (4.8%); | 33.9% |
| Fabry disease, with body weight ≥ 70 kg | [62.8, 77.4] | | [63.8, 76.8] | |
| Typical Q/F (L/h) for those with | 1.00 (5.17%); | — | 1.01 (4.59%); | — |
| body weight ≥ 70 kg | [0.899, 1.10] | | [0.928, 1.11] | |
| Typical V₃/F (L) for those with | 27.5 (11.7%); | — | 27.5 (11.9%); | — |
| body weight ≥ 70 kg | [21.2, 33.8] | | [22.7, 35.2] | |
| Kₐ (intercept) (h⁻¹) | 0.256 (9.41%); | 60.4% | 0.256 (8.60%); | 59.9% |
| | [0.209, 0.303] | | [0.211, 0.298] | |
| Kₐ (slope) | 0.284 (9.12%); | 60.7% | 0.282 (7.45%); | 60.6% |
| | [0.233, 0.335] | | [0.244, 0.326] | |
| Lag time (h) | 0.175 (4.65%); | | 0.176 (4.52%); | — |
| | [0.159, 0.191] | | [0.160, 0.190] | |
| WT-related exponential index on | Fixed to 0.75 | — | Fixed to 0.75 | — |
| CL/F and Q/F for those with body | | | | |
| wight < 70 kg | | | | |
| WT-related exponential index on | Fixed to 1 | — | Fixed to 1 | — |
| V₂/F and V₃/F for those with body | | | | |
| wight < 70 kg | | | | |
| Fractional Change in V₂/F in | −0.306 (12.8%); | — | −0.305 (12.3%); | — |
| subjects without Fabry disease | [−0.383, −0.229] | | [−0.372, −0.227] | |
| (decrease in V₂/F) | | | | |
| Fractional Change in total CL/F | 0.150 (24.9%); | — | −0.151 (23.5%); | — |
| in subjects without Fabry disease | [−0.223, −0.077] | | [−0.233, −0.081] | |
| (decrease in total CL/F) | | | | |
| Residual Error (%) | 26.2%; | — | 26.3%; | — |
| | [23.2%, 29.0%] | | [24.5%, 27.8%] | |
| Residual Error (ng/mL) | 2.55; | — | 2.47; | — |
| | [NA, 3.76] | | [1.25, 3.51] | |

^{a} Derived total CL/F parameter from typical EGFR-related estimate and EGFR-related exponential index; total CL/F = THETA(1)\hat{ }THETA(9), where THETA(1) is the typical EGFR-related estimate and THETA(9) is the estimate of exponential index for patients with Fabry disease, EGFR = 90 mL/min/1.73 m², and with body weight ≥ 70 kg.
^{b} Derived total CL/F parameter from typical EGFR-related estimate and EGFR-related exponential index; total CL/F = THETA(13)\hat{ }THETA(9), where THETA(13) is the typical EGFR-related estimate and THETA(9) is the estimate of exponential index for patients with Fabry disease, EGFR > 120 mL/min/1.73 m², and with body weight ≥ 70 kg.

The estimated parameters from bootstrap (see Table 6) were nearly identical to those estimated from the original dataset. All parameters were estimated with adequate precision. The NONMEM estimates (which assume each parameter has a normal distribution) were nearly identical to the nonparametric bootstrap estimates (which do not assume that each parameter has a normal distribution).

Model performance comparison was made for the adult population. Simulations were performed using a simulated adult dataset following 150 mg of migalastat salt QOD doses with both model parameters and the steady-state AUCtau and Cmax were compared. The results showed in Table 7 were comparable between the original model and the optimized/updated model, indicating a good model performance.

TABLE 7
Comparison between the original model and optimized
model with simulation results for adults receiving
150 mg of migalastat salt QOD dose.

| | |
|---|---|
| Cₘₐₓ (nm/mL) | AUCₜₐᵤ |

| | |
|---|---|
| Model | Geometric Mean (CV % of Geometric Mean) |

| | | |
|---|---|---|
| Original Model | 1120 (34.5%) | 7200 (32.5%) |
| (N = 100) | | |
| Optimized Model | 1120 (36.3%) | 7580 (32.0%) |
| (N = 100) | | |

Clinical trial simulations were then conducted to predict the exposure in pediatric patients receiving the initial various weight-based dosing regimens (comparable to about a 3 mg/kg dose). The dose regimens that were used for the simulations are listed in Table 8.

TABLE 8
Dose Regimen for Pediatric Patients.

| Weight (kg) | Dose (mg) | Frequency |
|---|---|---|
| <15 | 25 | QOD |
| 15 to <25 | 50 | QOD |
| 25 to <35 | 75 | QOD |
| 35 to <50 | 100 | QOD |
| ≥50 | 150 | QOD |

The doses were targeted to achieve a similar AUClau at steady-state (and not Cₘₐₓ or Cₘᵢₙ) in pediatric sub-groups to that in adults with normal renal function receiving 150 mg of migalastat salt every other day (QOD).

The pediatric simulations assumed the following: (1) 100 subjects per group for 4 groups including 3 pediatric groups with Fabry disease (2 to <6, 6 to <12 and 12 to <18 years) and 1 adult group (Fabry disease with normal renal function), assuming 50% males and 50% females in each group; (2) All children (and adults) had a normal renal function; (3) Age for pediatric subjects was sampled from a uniform distribution within the age limit of each group; (4) Weight for pediatric subjects was sampled from the normal distribution using the World Health Organization (WHO) weight chart for age for those less than 5.08 yrs., and from the Centers for Disease Control and Prevention (CDC) weight chart for those between 5.08 and 17.99 year old; and (5) The weight of the adult group was sampled from a random normal distribution (mean=75, standard deviation (SD)=15).

The results of the simulations, which are shown in Table 9, showed that the Cₘₐₓ values were comparable among groups, whereas the AUCₜₐᵤ (0-48 hrs) was about 25% lower in age group 2 to <6 year old (5570 vs 7580 h*ng/ml), and about 10% lower in age group 6 to <12 year old (6850 vs 7580 h*ng/ml).

TABLE 9
Pediatric Study Design with Empirical Dose Scheme PK Parameters.

| | Cₘₐₓ | AUCₜₐᵤ | CL/F | QOD | Tₘₐₓ |
|---|---|---|---|---|---|
| | (ng/mL) | (h*ng/mL) | (L/h) | Dose | (hrs) |
| | Geometric Mean (CV % of Geomean) | | | (mg) | Median |
| Groups | [95% CI] of Geometric Mean Parameter | | | Frequency | (Min, Max) |
| 2 to <6 Years | 1030 (38.6%) | 5570 (37.9%) | 5.56 (37%) | 25 mg (N = 40) | 2 |
| N = 100 | [490, 2150] | [2700, 11500] | [2.73, 11.3] | 50 mg (N = 60) | (1-4) |
| 6 to <12 Years | 1100 (37.4%) | 6850 (34.0%) | 8.52 (38.3%) | 50 mg (N = 36) | 3 |
| N = 100 | [536, 2250] | [3550, 13200] | [4.09, 17.8] | 75 mg (N = 37) | (1-4) |
| | | | | 100 mg (N = 22) | |
| | | | | 150 mg (N = 5) | |
| 12 to <18 Years | 1190 (40.1%) | 7530 (37.5%) | 14.1 (39.4%) | 75 mg (N = 2) | 2 |
| N = 100 | [553, 2560] | [3670, 15500] | [6.64, 29.9] | 100 mg (N = 32) | (1-4) |
| | | | | 150 mg (N = 66) | |
| Adults | 1120 (36.3%) | 7580 (32.0%) | 16.2 (32.0%) | 150 mg | 3 |
| N = 100 | [556, 2250] | [4090, 14100] | [8.70, 30.0] | (N = 100) | (2-5) |

A weight range analysis with a 5 kg increment on the simulated data was applied, which are shown in Table 10. Using the AUCₜₐᵤ geometric mean value of adult group with normal renal function receiving 150 mg QOD dose as the target (7580 h*ng/ml), dose adjustment was performed for subjects in each weight group considering dose proportionality with the equation 1:

$${Dose}_{adj , i} = {Dose}_{org , i} * {AUC}_{tau , a} / {AUC}_{tau , i} \qquad Equation 1$$

where Dose_{adj.i} is the adjusted dose for each weight group for achieving equivalent AUC exposure with adults, Dose_{org,i} is the original dose used for each weight group, AUC_{tau,a} is the adult group geometric mean value of 7580 h*ng/ml, and AUC_{tau,i} is the geometric mean value for each weight group. Additionally, the adjusted doses were rounded to the nearest practical dose level to ensure simplicity in formulation preparation.

TABLE 10
Pediatric Dose Adjustment Per 5 kg Weight Range.

| | Geometric | | | Adjusted | Adjusted |
|---|---|---|---|---|---|
| Weight | Mean | Number | Original | Dose | Dose |
| Range | AUCₜₐᵤ | of | Dose | Calculated | Round |
| (kg) | (h*ng/mL) | Subjects | (mg) | (mg) | (mg) |
| 10-15 | 4481 | 40 | 25 | 42 | 40 |
| 15-20 | 6658 | 52 | 50 | 57 | 60 |
| 20-25 | 5673 | 43 | 50 | 67 | 60 |
| 25-30 | 7413 | 21 | 75 | 77 | 80 |
| 30-35 | 7230 | 19 | 75 | 79 | 80 |
| 35-40 | 8040 | 18 | 100 | 94 | 100 |
| 40-45 | 7817 | 18 | 100 | 97 | 100 |
| 45-50 | 5904 | 18 | 100 | 128 | 150 |
| 50+ | 7907 | 71 | 150 | 144 | 150 |

The resulted adjusted dosing scheme for pediatric groups are summarized in Table 11.

TABLE 11
Summary of Adjusted Dosing Scheme for Pediatric Groups

| Weight (kg) | Dose (mg) | Frequency |
|---|---|---|
| <15 | 40 | QOD |
| 15 to <25 | 60 | QOD |
| 25 to <35 | 80 | QOD |
| 35 to <45 | 100 | QOD |
| ≥45 | 150 | QOD |

Based on the dose adjustment analysis and the new revised dosing scheme, simulations were re-run for the 3 pediatric groups (pediatric group age 2 to <6, 6 to <12 and 12 to <18 years), with all other assumptions and settings unchanged, the results of which are shown in Table 12.

TABLE 12
Predicted migalastat in pediatrics based on proposed weight-based dosing scheme.

| | Cₘₐₓ | AUCₜₐᵤ | CL/F | QOD | Tₘₐₓ |
|---|---|---|---|---|---|
| | (ng/mL) | (h*ng/mL) | (L/h) | Dose | (hrs) |
| | Geometric Mean (CV % of Geomean) | | | (mg) | Median |
| Groups | [95% CI] of Geometric Mean Parameter | | | Frequency | (Min, Max) |
| 2 to <6 Years | 1400 (36.8%) | 7540 (35.7%) | 5.66 (32.1%) | 40 mg (N = 37) | 2 |
| N = 100 | [691, 2840] | [3790, 15000] | [3.04, 10.5] | 60 mg (N = 59) | (1-4) |
| | | | | 80 mg (N = 4) | |
| 6 to <12 Years | 1230 (36.9%) | 7660 (33.1%) | 8.96 (38.6%) | 80 mg (N = 21) | 3 |
| N = 100 | [606, 2500] | [4040, 14500] | [4.28, 18.8] | 80 mg (N = 48) | (1-4) |
| | | | | 100 mg (N = 21) | |
| | | | | 150 mg (N = 10) | |
| 12 to <18 Years | 1250 (36.6%) | 7870 (32.1%) | 14.1 (38.2%) | 80 mg (N = 3) | 2 |
| N = 100 | [616, 2520] | [4230, 14600] | [6.77, 29.3] | 100 mg (N = 20) | (1-4) |
| | | | | 150 mg (N = 77) | |
| Adults | 1120 (36.3%) | 7580 (32.0%) | 16.2 (32.0%) | 150 mg | 3 |
| N = 100 | [556, 2250] | [4090, 14100] | [8.70, 30.0] | (N = 100) | (2-5) |

population PK data in adults and adolescents weighing >45 kg receiving the 150 mg migalastat HCL capsule q.o.d. are presented in Table 13.

TABLE 13
Simulated pharmacokinetic endpoints
by age groups and adults ≥ 45 kg.

| Age Group | Cₘₐₓ | Cₘᵢₙ | AUCₜₐᵤ |
|---|---|---|---|
| (Years) | (ng/mL) | (ng/mL) | (h*ng/mL) |
| 12 to <16 | 1377 (42%) | 8.06 (37%) | 8581 (37%) |
| 16 to <18 | 1275 (39%) | 8.37 (38%) | 8408 (37%) |
| 12 to <18 | 1319 (41%) | 8.23 (37%) | 8483 (37%) |
| Adults | 1191 (37%) | 8.13 (41%) | 7958 (35%) |

Abbreviations: AUC₀₋ₜₐᵤ = plasma concentration-time curve during a dosing interval at steady state (AUC_{0-τ}); Cₘₐₓ = maximum observed plasma concentration; Cₘᵢₙ = minimum observed plasma concentration;
Note:
Data are summarized as geometric mean (CV %)

Results of ANOVA analysis are presented in Table 14.

TABLE 14
Summary of the ANOVA on predicted pharmacokinetic
parameters for subjects weighing ≥ 45 kg.

| PK Endpoint | Age Group (Years) | Point Estimate (90% CI) |
|---|---|---|
| AUC₀₋ₜₐᵤ | 12 to <16 | 108 (98.6, 118) |
| | 16 to <18 | 106 (97.1, 115) |
| | 12 to <18 | 107 (99.0, 115) |
| Cₘₐₓ | 12 to <16 | 116 (105, 127) |
| | 16 to <18 | 107 (97.6, 117) |
| | 12 to <18 | 111 (102, 120) |

Abbreviations: AUC₀₋ₜₐᵤ = plasma concentration-time curve during a dosing interval at steady state; CI = confidence interval; Cₘₐₓ = maximum observed plasma concentration
The limited pharmacokinetic data support the 150 mg migalastat HCL capsule Q.O.D. dose in adolescents weighing ≥ 45 kg.

### Example 3: PK/PD Model Validation in Adolescents

The example describes AT1001-020 study, which is an Open-label Study of the Safety, Pharmacokinetics, and Pharmacodynamics of Migalastat in Pediatric Subjects (aged 12 to <18 years) with Fabry Disease and Amenable GLA Variants.

The disclosure includes analysis of interim clinical study data, presenting the results of the stage 1 (1-month) safety and PK data only for subjects with Fabry disease in the 12 to <16 years old age group who had Stage 1 plasma concentration-time data available as of the cut-off date.

### Objectives

Stage 1 objective is to characterize the PK of migalastat in adolescents with Fabry disease, and to validate extrapolation of migalastat plasma exposure in adults to adolescents weighing ≥ 45 kg for the 123 mg migalastat capsule administered once every other day (QOD).

Another Stage 1 objective is to evaluate the safety of migalastat treatment in pediatric subjects with Fabry disease and who have variants in the gene encoding α-Gal A (GLA) amenable to treatment with migalastat.

### Outcomes/Endpoints

Pharmacokinetic Endpoints were as follows:
Population PK model that describes the relationship between weight and age and migalastat pharmacokinetics in pediatric subjects (with primary PK parameter outputs listed in the following text).
PK parameters based on simulated plasma-concentration data for migalastat after multiple-dose administration at steady-state concentration
Cₘₐₓ: maximum observed plasma concentration
Cₘᵢₙ: minimum observed plasma concentration
tₘₐₓ: time to reach Cₘₐₓ
AUC₀₋ₜₐᵤ: area under the plasma concentration-time curve from time 0 over the dosing interval (i.e. 48 hours)
t_{1/2}: terminal elimination half-life
CLₛₛ/F: apparent oral clearance at steady-state concentration
Vₛₛ/F: apparent oral volume of distribution at steady-state concentration

### Study Participants

The disclosure describes the PK/PD study in migalastat-treated patients who were either naïve to enzyme replacement therapy (ERT) or had stopped ERT at least 14 days at the time of screening

For inclusion in this study, subjects must have met all of the following criteria:
Male or female, diagnosed with Fabry disease aged between 12 and <18 years at baseline, and who might benefit from specific treatment for their condition, in the opinion of the investigator.
Confirmed, amenable GLA variant determined using the migalastat amenability assay (For subjects without a known amenable GLA variant, GLA genotyping must have been performed prior to Visit 2. Similarly, For subjects with a GLA variant that had not yet been tested in the migalastat amenability assay, amenability testing must have been completed before Visit 2).
Weight of >45 kg (99 pounds) at screening.
Treatment-naïve or discontinued ERT treatment at least 14 days prior to screening 5. Had at least one complication (i.e. historical or current laboratory abnormality and/or sign/symptom) of Fabry disease.
Had no indication of moderate or severe renal impairment (estimated glomerular filtration rate [eGFR] <60 mL/min/1.73 m²) or kidney disease requiring dialysis or transplantation at screening.

### Treatment

One migalastat 123 mg migalastat (=150 mg migalastat HCL) capsule was administered to adolescents weighing >45 kg with water every other day during the study.

Due to the capsule size and inclusion criteria of study AT1001-020, the 123 mg migalastat capsules are not suitable for patients less than 45 kg body weight and for the lower weight and age groups. Thus, it was recommended to include a warning for the lower weight group within the proposed age group (12 to below 16 years).

Sparse sampling for plasma migalastat concentrations to estimate exposure was done at baseline and for one 24-hour period between days 15 and 30. As shown in Table 15, subjects were randomly assigned to one of the 3 PK sampling groups.

TABLE 15
Sparse sampling schedule in study AT1001-020.

| PK Sampling | Time Post-dose | | | |
|---|---|---|---|---|
| Group | Sample 1 | Sample 2 | Sample 3 | Sample 4 |
| 1 | 1 h to 1 h | 1 h 30 min | 5 h to 5 h | 6 h 30 min |
| | 15 min | to 2 h | 30 min | to 7 h |
| 2 | 1 h to 1 h | 2 h 45 min | 5 h 15 min | 10 h 45 min |
| | 15 min | to 3h | to 5 h | to 11 h |
| | | 15 min | 45 min | 15 min |
| 3 | 3 h 15 min | 3 h 45 min | 8 h 15 min | 8 h 45 min |
| | to 3 h | to 4 h | to 8 h | to 9 h |
| | 45 min | 15 min | 45 min | 15 min |

Patients with 1 plasma concentration-time data available as of the cut-off date were included in the interim analysis.

Plasma samples were analyzed using the LC-MS/MS method.

### Analysis Populations for Interim Analysis

The safety population included all subjects aged 12 to <16 years who received at least 1 dose or a partial dose of study drug and had Stage 1 plasma concentration-time data available as of the cut-off date. All safety analyses were performed using the safety population.

The PK population included data from subjects aged 12 to <16 years who have completed Stage 1 and who received at least 1 dose of migalastat with at least 1 quantifiable concentration. All subjects included in the Interim Analysis population PK had a known weight and an eGFR.

### Results

### Baseline Data

A total of 22 subjects were enrolled in the study AT1001-020. As of the cut-off date, a total of 9 subjects, 4 females and 5 males, aged 12 to <16 years were enrolled in Study AT1001-020, received study drug, and completed Stage 1 of the study with PK concentration data. They comprised the safety and PK populations for this interim analysis. The mean number of years since diagnosis of Fabry disease was 10.2 (+4.12) years. Four subjects reported prior use of enzyme replacement therapy.

The median duration of migalastat exposure for the 9 subjects enrolled in Study AT1001-020 was 30 days with maximum exposure of 49 days.

Demographics and baseline characteristics are presented in

[symbol]

TABLE 16
Demographics - Safety Population.

| Parameter | Statistic | Migalastat | |
|---|---|---|---|
| Number of Subjects in the | N | 9 | |
| safety population | | | |
| Age (years)^{a} | Mean (SD) | 14.1 | (1.17) |
| | Median | 15.0 | |
| | Min, Max | 12, | 15 |
| Sex | | | |
| Male | n (%) | 5 | (55.6) |
| Female | n (%) | 4 | (44.4) |
| Race | | | |
| White | n (%) | 8 | (88.9) |
| Black or African American | n (%) | 0 | |
| Asian | n (%) | 0 | |
| American Indian or | n (%) | 0 | |
| Alaska Native | | | |
| Naive Hawaiian or other | n (%) | 0 | |
| Pacific Islander | | | |
| Other | n (%) | 1 | (11.1) |
| Ethnicity | | | |
| Hispanic or Latino | n (%) | 2 | (22.2) |
| Not Hispanic or Latino | n (%) | 7 | (77.8) |
| Height | Mean (SD) | 167.09 | (5.591) |
| | Median | 168.50 | |
| | Min, Max | 160.0, | 175.3 |
| Weight (kg) | Mean (SD) | 67.56 | (17.273) |
| | Median | 66.50 | |
| | Min, Max | 45.0, | 100.6 |
| Body Mass Index | Mean (SD) | 24.25 | (6.148) |
| (kg/m²) | Median | 24.10 | |
| | Min, Max | 15.6, | 33.5 |

Abbreviations: Max = maximum; Min = minimum; N = total number of subjects; n = number of subjects in category indicated; SD = Standard deviation
Note:
Percentages are based on the number of subjects in the safety population.
^{a}Age = (informed consent date − date of birth + 1)/365.25 and truncated to complete years

TABLE 17
Baseline Characteristics - Safety Population.

| Parameter | Statistic | Migalastat | |
|---|---|---|---|
| Number of Subjects in the | N | 9 | |
| safety population | | | |
| Number of years since diagnosis | Mean (SD) | 10.15 | (4.119) |
| of Fabry disease ^{a} | Median | 11.17 | |
| | Min, Max | 3.4, | 15.8 |
| Previous use of ERT n (%) | | | |
| Yes | n (%) | 4 | (44.4) |
| No | n (%) | 5 | (55.6) |

Abbreviations: Max = maximum; Min = minimum; N = total number of subjects; n = number of subjects in category indicated; SD = Standard deviation
Note:
Percentages are based on the number of subjects in the safety population.
^{a} Age = (informed consent date − date of birth + 1)/365.25 and truncated to complete years

### Medical History

The most common system organ classes for medical history in the safety population were nervous system disorders (77.8%), ear and labyrinth disorders (66.7%), gastrointestinal disorders (66.7%), and general disorders and administration site conditions, investigations, psychiatric disorders, respiratory, thoracic and mediastinal disorders, and skin and subcutaneous tissue disorders (all 55.6%). The most common medical history preferred terms (all reported by 55.6% of the subjects) were tinnitus, abdominal pain, diarrhea, headache, and paranesthesia, most of which are consistent with Fabry disease.

### Prior and Concomitant Medications

All but 1 subject reported prior use of medications. The most common previous medication was paracetamol taken by 6 (66.7%) subjects. No other medication was taken by more than 2 subjects.

The most frequently used concomitant medication was paracetamol taken by 6 (66.7%) subjects. No other concomitant medication was taken by more than 2 subjects.

### Adverse Events

An overall summary of TEAEs experienced by subjects in the safety population during Stage 1 is displayed in Table 18 and Table 19.

TABLE 18
Summary of Treatment-emergent Adverse
Events - Safety Population - Stage 1.

| Parameter | Statistic | Migalastat |
|---|---|---|
| Number of subjects in the | N | 9 |
| safety population | | |
| Number of TEAEs | n | 6 |
| Number of subjects with TEAEs | n (%) | 5 (55.6) |
| Number of subjects with related TEAEs | n (%) | 1 (11.1) |
| Number of subjects with treatment- | n (%) | 0 |
| emergent SAEs | | |
| Number of subjects discontinued | n (%) | 0 |
| due to TEAEs | | |
| Number of subjects with AEs | n (%) | 0 |
| leading to death | | |

TABLE 19
Frequency of Treatment-emergent Adverse Events
Occurring in the Safety Population - Stage 1.

| System Organ Class | Number of | Number of |
|---|---|---|
| Preferred Term | Subjects n (%) | Events n (%) |
| Number of subjects with TEAEs | 5 (55.6) | 6 |
| Infections and infestations | 4 (44.4) | 4 (66.7) |
| Pharyngitis | 1 (11.1) | 1 (16.7) |
| Upper respiratory tract infection | 3 (33.3) | 3 (50.0) |
| Nervous system disorders | 1 (11.1) | 1 (16.7) |
| Headache | 1 (11.1) | 1 (16.7) |
| Skin and subcutaneous | 1 (11.1) | 1 (16.7) |
| tissue disorders | | |
| Drug eruption | 1 (11.1) | 1 (16.7) |

### Laboratory Findings

During Stage 1, urinalysis (albumin, protein, specific gravity, pH, and microscopy) was the only laboratory parameter collected at Month 1 and therefore, the only laboratory parameter assessed for the Interim Analysis.

There were no clinically meaningful changes in mean values from baseline for urinalysis parameters at Month 1.

There were a few shifts from baseline to Month 1. Three subjects had pH values that went from normal at baseline to high at Month 1.

There were no potentially clinically significant abnormalities in urinalysis parameters.

Urine pregnancy tests were performed for all female subjects of childbearing potential at every visit. No female subject in the safety population had a positive pregnancy test result during Stage 1.

### Conclusions on Clinical Safety

Based upon limited data obtained from adolescent patients aged 12-18 years (n=9), popPK data showed that exposure in adults and adolescents weighing >45 kg receiving the 123 mg migalastat capsule q.o.d. was comparable.

Cₘₐₓ levels observed in the pediatric patients were in line with the Cₘₐₓ levels observed in adults patients in the pivotal study AT1001-011.

No new safety findings have been observed during stage 1 of the study. Hence treatment with migalastat 123 mg in pediatric patients aged ≥12 to 16 years of age does not lead to a different safety profile than already known.

A new formulation, migalastat HCl oral formulation (sachet and/or capsules) for treatment of Fabry disease in pediatric and adolescent patients aged 2 to <18 years and with amenable GLA mutations may be designed and evaluated.

### Example 4: Clinical Efficacy of Migalastat Treatment in Adolescents

The example describes AT1001-020 study, which can be an Open-label Study of Efficacy of 12-month Treatment with Migalastat in Pediatric Subjects (aged 12 to <18 years) with Fabry Disease and Amenable GLA Variants. In some embodiments, the clinical efficacy study comprises stage 2.

Accordingly, in some embodiments, in Stage 2, Primary Objective can include evaluating the safety of migalastat treatment in pediatric subjects diagnosed with Fabry disease and who have GLA variants amenable to treatment with migalastat.

In some embodiments, in state 2, Secondary Objectives can include characterizing the pharmacodynamics (PD) of migalastat in pediatric subjects diagnosed with Fabry disease and who have GLA variants amenable to treatment with migalastat.

In some embodiments, in state 2, secondary objective can include evaluating the efficacy of migalastat in pediatric patients diagnosed with Fabry disease and who have GLA variants amenable to treatment with migalastat.

In some embodiments, in state 2, secondary objective can include evaluating the relationship between exposure to migalastat and response.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

The embodiments described herein are intended to be illustrative of the present compositions and methods and are not intended to limit the scope of the present invention. Various modifications and changes consistent with the description as a whole and which are readily apparent to the person of skill in the art are intended to be included. The appended claims should not be limited by the specific embodiments set forth in the examples, but should be given the broadest interpretation consistent with the description as a whole.

Patents, patent applications, publications, product descriptions, GenBank Accession Numbers, and protocols are cited throughout this application, the disclosures of which are incorporated herein by reference in their entireties for all purposes.

What is claimed is:

1. A method of treatment of Fabry disease in a human patient in need thereof, the method comprising administering to the patient a formulation comprising therapeutically effective dose of migalastat or a salt thereof, wherein the patient is a pediatric patient.

2. The method of claim 1, wherein the patient has an age in a range of from about 2 year to about <18 year.

3. The method of claim 1, wherein the patient has a weight in a range of from about <15 kg to about ≥50 kg.

4. The method of any one of claims 1-3, wherein the therapeutically effective dose of migalastat or a salt thereof is in a range of from about 15 mg to about 150 mg every other day.

5. The method of any one of claims 1-4, wherein the therapeutically effective dose of migalastat hydrochloride at a dose in a range of from about 25 mg to about 150 mg every other day.

6. The method of any one of claims 1-5, wherein the therapeutically effective dose of migalastat FBE in a range of from about 15 mg to about 123 mg every other day.

7. The method of claim 1 or 2, wherein the patient has an age in a range of from about 12 year to about <18 year.

8. The method of claim 7, wherein the patient has a weight of about ≥25 kg.

9. The method of claim 8, wherein the therapeutically effective dose of migalastat hydrochloride is in a range of from about 80 mg to about 150 mg every other day.

10. The method of claim 7, wherein the patient has a weight of about >45 kg.

11. The method of claim 10, wherein the therapeutically effective dose of migalastat hydrochloride is about 150 mg every other day.

12. The method of claim 10 or 11, wherein the therapeutically effective dose of migalastat FBE is about 123 mg every other day.

13. The method of claim 1 or 2, wherein the patient has an age in a range of from 6 year to <12 year.

14. The method of claim 13, wherein the patient has a weight of about >25 kg.

15. The method of claim 13 or 14, wherein the therapeutically effective dose of migalastat hydrochloride is in a range of from about 80 mg to about 150 mg every other day.

16. The method of claim 1 or 2, wherein the patient has an age in a range of from 2 year to <6 year.

17. The method of claim 16, wherein the patient has a weight of about <35 kg.

18. The method of claim 16 or 17, wherein the therapeutically effective dose of migalastat hydrochloride is in a range of from about 40 mg to about 80 mg every other day.

19. The method of any one of claims 1-18, wherein the patient has an eGFR of about ≥60 mL/min/1.73 m².

20. The method of any one of claims 1-19, wherein the migalastat or salt thereof enhances or prolongs α-galactosidase A activity.

21. The method of any one of claims 1-20, wherein the formulation comprises an oral dosage form.

22. The method of claim 21, wherein the oral dosage form comprises a tablet, a capsule or a solution.

23. The method of any one of claims 1-22, wherein the patient is male.

24. The method of any one of claims 1-22, wherein the patient is female.

25. The method of any one of claims 1-24, wherein the patient is an enzyme replacement therapy (ERT)-naïve patient.

26. The method of any one of claims 1-25, wherein the patient is an ERT-experienced patient who has stopped ERT for at least 14 days.

27. The method of any one of claims 1-26, wherein the patient has a HEK assay amenable mutation in α-galactosidase A.

28. The method of claim 27, wherein the mutation is disclosed in a pharmacological reference table.

29. The method of claim 28, wherein the pharmacological reference table is provided in a product label for a migalastat product approved for the treatment of Fabry disease.

30. The method of claim 29, wherein the pharmacological reference table is provided in a product label for GALAPOLD®.

31. The method of claim 30, wherein the pharmacological reference table is provided at a website.

32. The method of claim 31, wherein the website is one or more of www.galafoldamenabilitytable.com or www.fabrygenevariantsearch.com.


SEQUENCE LISTING (3 sequences)

SEQ ID NO: 1 - DNA, 12436 bp, Homo sapiens
  source 1..12436: mol_type = genomic DNA
cccttctgtaggggcagagaggttctacttcattactgcgtctcctgggaaggccatcaggactgctggctaaagtgggaaccaggactctttgtgagttaagaatttgtgtatttatatgtgtgttatacacattttttaaaaaactgtaacgacatcaggttgagcagtcgtctccgggtggtgaattatgtgtatttttaaattttatactatattgttatttttcaaatgttcgaaattgaatatgtagattgttgttatcagcagaaaaataaacattattcaaatactctattcagtaaagtaatttattgggcgcctttgtcaagcacgcatttgcctagatgtgactctacagataaaattcacttggggcctccccttacagacaatcaggcagtggagactgagtgcctgaatggatagaccagcactcagaccactattttcagtatctgtttttcttaactcagggccgtggttttcaaacgtttttcgccttacggtcacccttagggtcccccgagaccggcccagacagacagatatacaaaaacacatacacagtcatgagcgtccaccatttccccaccaggcgcagcacaggcggcttcccggcactgagatgggggggaggagggagagagcgcgaggggggaggggaaagcagagaacgaaagaggcggaggcggcccccgaaccccgctctggtcttcatcatcaccacccctgggtccccagttcccacccacacaccaacctctaacgataccgggtaattttcctccttcttccctcaaacggctatagcgagacggtagacgacgaccagaactacttctgctcacgtaagcgagtaatcacgtgagcgcctacgtcatgtgagatctcggtcacgtgagcaactctcggcttaaactcgggatcactaaggtgccgcacttccttctggtatggaaatagggcgggtcaatatcaagaaaggaagagggtgattggttagcggaacgtcttacgtgactgattattggtctacctctggggataaccgtcccagttgccagagaaacaataacgtcattatttaataagtcatcggtgattggtccgcccctgaggttaatcttaaaagcccaggttacccgcggaaatttatgctgtccggtcaccgtgacaatgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcaggtatcagatattgggtactcccttccctttgcttttccatgtgtttgggtgtgtttggggaactggagagtctcaacgggaacagttgagcccgagggagagctcccccacccgactctgctgctgcttttttatccccagcaaactgtcccgaatcaggactagccctaaactttctctgtgtgacctttcctgggatgggagtccggccagcggcccctgtttctttctctctctctctctctctcgttctccttctctttctctttctcttctttcctctctctttctctctctccctgcccggttctcttttttcactgctccttgcagagcagggccaccccataggcagtgtgcccaaagtagccctgcccggttctattcagacccttcttgtgaacttctgctcttcctctgccgggtgctaaccgttagaacatctagggtgggtaggaggaatggggaactaagattcgtgccattttttctccttttggggtcgtggatttctcggcagtatctcgagggagttagagagaccataaggtcgctgagatctctcccacctcgcccatgagcgtggcatcaggctggaaggttgacatggaggaactttatacatttacacctttgcgtgagggttgaggctggattagataggtattgaacatatctgaccctcacaatccttatctgtaaattgggattacaaccttttaatttcagggagctgacaaaaaaaatctgaaaaatagttcttatctcacacaggtgagttttcaaggagataacctatttaaagtacatagcacagcgcttgaccattcaactgcgcttacagagcaaatgttcaatgggaaaatgaatgtaaatctacaaatctgaatgaatatgtgtatttttctggagagaggatatttacctttcttcaaattctcaaagggctctgtgatttaaaaaaggttaggaatcactgatagatgttggtaaaaggtggcagtcacagtacatttctgtgtccataagttattcctatgaatatctttatagataaagtcaggatgttggtcagacatcacagaagaaattggccttgtaagtttcatgtgaccctgtggtacagtatgtgtggcaattttgcccatcacggatttttttttattggtatttgcatctgattataaaactaatgcatgatcattgcaaaaaatgtagataaagaagagcaaaatgaaaataaagatttccccccaccgttccaccacccagaaataatcatggtttaaatgttaatatacaaccttacaattgttttctatataaatgaaaacatagatttctttatttcattattttccataaaaaatggatcatgtttatgtcatgtttggctaatggcaagaccctggcacccagtctgggctcaaattctgcctcattgttacttagccctgtgacattgggtaaattacactttttttttttttttttttttgagacggggtctcgctctgtcgcccaggctggagtgcagtggcacgatctcggctcactgcaagtccgcctcctgggttcacgccattcttctgcctcagcctcccgagtagctgggactacaggcgcctgccaccacgcctggctctttttttttttttttttttttttagtacagacggggtttcaccatgttagccagggtggtctcaatctcctgacctcgtgattcgcccgcctcagcctcccaaagtgctggtgtgagccaccgtgcccagccttacttttttttttgagagggggtctcactctgtcacccaggttggagtgcagtggcgcgatctctgctcagtgcaaactccacctcccgggtttaagcagttctcctgtcgtagtctcctgagtagctgggattacaggcacaccaccacggccagctaatttttgtattttcagtagagacgggtttcaccatgttgcccaagctggtctcgaactcctggcctcaagtgatctgcccgccttggcctcccagagtgctgggattacaggtgtgagccaccgcacccggcctcttttttcttttttagtctatcataccttgcaaatacagtggttcttcctatgtgttggttttgatatttatgtaatcaaacacatcagtttttcctttctgatttctgactttggggtcatgctgagaaagtcctttcctacctgaagataatacagtatatacgtttcttactagtatttttgtggatttttaaaatatttaaatctttagtccatctgaacttgttcttctatcagaaatgccacatttaataaataataagtcccatggtatcagatggctggaaggacctctttcgaaactttgtttaattccattaatctgtgtattcttattctaatgctaatagttccacactagcttcctttatcttttttttctttttttttttttttttgagctggagtttcgctcttgttgcccaggctggagtacaatgtcacgatctcggttcaccgcaacctccgcctcccaggttcaagcaattctcctgcctcatcctcgcgagtagctggaattacaggcatgcgccaccacgcctagctattttgtatttttagtagagatggggtttctccatgttggtcaggctggtctcaaactcccagcctcaggtgatctgcctgcctcggcctcccaaaatgctgttattacaggcgtgagccaccacgcccagccttcatcttttaatgaatgtacatgtatgtaatcttttaggtgaactttttgtaatgttgtgccaagttccttaaaaagcccttttggaagctgggcaggtggccacgcctgtaatcccagcattttgggagtctgaggcaggtggatcacttgaggccaggagttcaagactagcctagccaaaatgcaaaaccctgtctctactaaagatacaaaaattagccggatgcgatggcacatgcctgtaatctcagctactcgggaggctgaggtagaagaatcgcttgaaccggggaggcagaggttgcagtgagcaagatggcgccactgcactccagcctgggtgacagagggagactccatctcaaaaaaaaaaaaaaaaaaaaagataaaaaggaaacctaagtactcttgggctttgttaaggattttgttaaatatacaaaggattgcagggaaaattaacttatttttaatattgagtatgcttatccaagagcaaaataatatttctccatttattcaaatcatttaggagcatcatagttttaacatatgggccttgcacgtatcttaaatttatctctaggcattttaggttgttcagttgttcttgtgaatgggatctttttctccaaataggattattgttgatatctgttgattatgttaactttgtagtttctgactttactgaactgtcttcttagatctaatactcttttcaatttcatcatatatttctcattcctattttgtttggggtttttagggcgggaatattaacgggataagagagacaaaagaaaatctggaaaaacaattcattttaccttacattgcttgtgattactaccacactattactgggttggaaaaaattgtgaaatcccaaggtgcctaataaatgggaggtacctaagtgttcatttaatgaattgtaatgattattggaatttctctttcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgtgagtttatagataatgttcttgttcattcagaggactgtaagcacttctgtacagaagcttgtttagaaacagccctcatggccgggcgtggtggctcacgctgtaatcccaacactttgggaggccgaggcgggtggatcacctgaggtcaagagttcaagaccagcctggccaacatggtgaaaccccaactctattaaaagtacaaaaaattagctgggcatggtggtgaacgcctgtaaccccagctacttgggaggctgaggcaggagaatcgcttgaacccaggaggtggaagtttcagtgagctgagatcacgccattgcactctagcctgggcaacaaaagagaaactccatctcaaaaaaaaaaacaaggaaaaaaagaaacagccctcatgacacttagaaagtagaatagctggctgttatctgaacattgaattgtaaggcttatcaggtggactttgcattccatcagcagacaatttttttttttttttttttttgagatggagtctcattctgtctcccaggctggagggcagtggtgcgatctcggctcactgcaagctccacctcctgggttcatgccattctcctgcctcagcctcccaagtagctgggaccacaggcacccgccaccatgcccagttaattttttgtatttttagtagagacggggtttcaccatgttagccaagatggtctcgatctcctgacctcgtgatccgcccacctcggcctcccaaagtgctgggattacaggcatgagccaccgcgcctagcctacaaatgttttgtaatagctcttgaggcccatcttggagttctccttttgctaaaaccactgaactctctaggaggaaaaaggaacttggttcttgacatatgtgtgcatgtatttccatataacctttaggaagctattgcaatggtactataaactagaattttagaagatagaaggaaaatattctggagatcattgaagagaaatggagtccaacactagttaaagatgatgaagacagatttttttttttgacggagtctcgctctgtcgcccaggctggagtgcagtggcacaatctcagctcactgcaaccctccacctcttgggttcaagtgattctcctgcctcagcctcccaagtagctgggactacaggcgcacaccaccacgcccggctaatttttgtatttttagtagagacaaggtttcaccatattcgccaggctggtctcgaactcctgaccttgtaatccgcccaccttggcctcccaaagtgctgggattacaggcatgagccaccacgcccggccgatgaagacagattttattcagtactaccacagtagaggaaagagccaagttcaattccaaatacaacaaagacaggtggagatttatagccaatgagcagattgagggggtcagtggatggaatatttaagaagacatcaagggtagggagcttcttgctaaagcttcatgtacttaaacaagaagggtgggggatgagggaaattgatcagatatcaatggtggcagtattgacttagcaggattcttgctaagaggtcttgctaggacagacataggaagccaaggtggaggtctagtcgaaaagaaggctcatcagagaagtctaactaaagtttggtcaagaagagtctttgtcaaggtaaatctatcatttccctcaaaaggtaattttcaggatcccatcaggaagattagcatggctgctagctttctcctcagttctgggctatagctcacatgcctagtttgaactagctcagcagaactgggggatttattctttgtcttccaacaaactcatctggatgattttgggggtttgtggggaaaagcccccaatacctggtgaagtaaccttgtctcttcccccagcctggaatggttctctctttctgctacctcacgattgtgcttctacaatggtgactcttttcctccctctcatttcaggttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggtaatgtttcattccagagatttagccacaaaggaaagaactttgaggccatggtagctgagccaaagaaccaatcttcagaattttaaataccctgtcacaatactggaaataattattctccatgtgccagagctcccatctcttctctttcagttcattaattaattaattaattcatgtaaaatccatgcatacctaaccatagctaatattgtgcacttataattcaagagggctctaagagttaattagtaattgtaactctctataacatcatttaggggagtccaggttgtcaatcggtcacagagaaagaagcatcttcattcctgcctttcctcaatatacacaccatctctgcactacttcctcagaacaatcccagcagtctgggaggtactttacacaatttaagcacagagcaactgcctgtccctgctgctagtttaaacatgaaccttccaggtagcctcttcttaaaatatacagccccagctgggcatgatggctcatgcctgtaatcctagcactttgggaggctgaggcgggtggattacttgaggtcaggagttcgagaccaccctggccaacatggtgaaaccccatctctagtaaaaatacaaaaattagctgactttggtggcacatgcctgtaatcccagctacttgggaagctgagacagaagagtcacttgaacctgggaaacagaggttgcagtgagccaagatcgcaccactgcactccaccctggatgacagactgaaccccatctcaaaaaattaaaataaaataaaataaaataactatatatatagccccagctggaaattcatttctttcccttattttacccattgttttctcatacaggttataagcacatgtccttggccctgaataggactggcagaagcattgtgtactcctgtgagtggcctctttatatgtggccctttcaaaaggtgagatagtgagcccagaatccaatagaactgtactgatagatagaacttgacaacaaaggaaaccaaggtctccttcaaagtccaacgttacttactatcatcctaccatctctcccaggttccaaccacttctcaccatccccactgctgtaattatagcctaagctaccatcacctggaaagtcatccttgtgtcttcccctttatttcaccattcatgtcctgtctatcaacagtccttccaccagtatctctaaaatatctcctgaatcagcccacttccttccatcttcactacatgcaccctggccttccaagctactatcggctctcaaccagactgctgggaccacctgatctctctgcttccactctgtctcaacccccatctattttccaagcagcactagagttatcatattaaaatgtaaatatcagttttttttttaaagaaaaaaaccctgagacttaacagagttataaaaaatataaatgtcatcatcagttccctgcttaaaacccttaactcgcttccaattgcacttggaatgaaaccaaactgcactgatccagcccttgcctgcctccccaaagtccaaggggtcatggctctttccctggctacactggttttctttctgtccctcaacactgcaagcctattgctgccccagggcctttacacttgctttttttctgcctagaacagttcttccccaaagatttttaaagggccgggctccttaacattgaagtcgcagaccaaacgccacatatgcagacagttcttctctaactactttaaaatagccctctgtccattcattcttcatcacattaacctgtttaattttcttctcagagctccacactatttggaagtatttgttgacttgttaccatgtctccccactagagtgtaagtttcatgagggcagggaccttgtctgactttgactgtatctctcgcatatggttaagtgttaaatagttatttatggaatgaatccctattattccctcattatctctgcaaaatagtcttttttctcaacatcttaaacctgatatcccacctgcctatctacaaactttttttttgcgacagagtctcactgtcacccaggctagagtgcagtggcgccatctcggctcactgcaacctccgcctcccgggtttaagcgattctcttgcctcagcctcccagtagctgggattataggcgtgcgctaccacatctggctaatttttgtatttttagtagagatggtttcaccatgttggccaggcttgtctcgaactcctgacctcagatgatccacctgcctcggcctcccaaagtgctgggattacaggcatgagccaccgtgcccagcctctacaaactttttattccattaacaaactatatgctgggatttaagttttcttaatacttgatggagtcctatgtaattttcgagcttttaattttactaagaccattttagttctgattatagaagtaaattaactttaagggatttcaagttatatggcctacttctgaagcaaacttcttacagtgaaaattcattataagggtttagacctccttatggagacgttcaatctgtaaactcaagagaaggctacaagtgcctcctttaaactgttttcatctcacaaggatgttagtagaaagtaaacagaagagtcatatctgttttcacagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatggtaaaaacttgagccctccttgttcaagaccctgcggtaggcttgtttcctattttgacattcaaggtaaatacaggtaaagttcctgggaggaggctttatgtgagagtacttagagcaggatgctgtggaaagtggtttctccatatgggtcatctaggtaactttaagaatgtttcctcctctcttgtttgaattatttcattctttttctcagttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacaggtaaataagagtatatattttaagatggctttatatacccaataccaactttgtcttgggcctaaatctatttttttcccttgctcttgatgttactatcagtaataaagcttcttgctagaaacattactttatttccaaaataatgctacaggatcattttaatttttcctacaagtgcttgatagttctgacattaagaatgaatgccaaactaacagggccacttatcactagttgctaagcaaccacactttcttggtttttcagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaaaatgtttattttattgccaactactacttcctgtccacctttttctccattcactttaaaagctcaaggctaggtggctcatgcctgtaatcccagcactttgggaggctgaggcgggcagatcacctgaggtcgggactttgagacccgcctggacaacatggtgaaaccccatttctaataaaaatataaaaattagccaggtgtggtggcgcacctgtggtcccagctactctgggggctgaggcatgagaatcgcttgaacccgggagtggaggttgcattgagctgagatcatgccacctcactccagcctgggcaacaaagattccatctcaaaaaaaaaaaaaaagccaggcacagtggctcatgcctggaatcccagcacttttggaagctgaggcaggcagatcacttgaggttaggatttcaagaccagcctggctaacatagtaaagccctgtctctactaaaaatacaaaaattagccaggtatggtggcgagcttctgtagccccagctactcaggagactgaggcaggagaatcacttgaacccgggaagtgggggggtgcagtgacccaagatcacgccactgcattccagcctgggcaacagagcaagactccatctcaaaaaaaaaagttctatttccttgaataaaattttccgaagtttaaactttaggaataaaactattaaacccgtatttactcatccagatacccaccccccttgttgagattctctcccaattatcaaaatgtgtagcatatttaactaccaagagctaaacatcattaagactgaaatgtattaagaaggatgtataggccaggcacggtgtctcacgcctgtaatcccaacactttgggaggccaagtcgggcggatcacgaggtcaggagatggagaccatcctggccaacatggtgaaaccccctctctactaaaaatacaaaaattagccaggcaggtggcaggcacctgtaatcccagctactccagaggctgaggcaggacaatcacttgaacctgggaggcagaggctgcagtgagctgaggttgtaccaattgcactccagcctaggtaacgagcaacactccatctcaaaaaaagaaaaaaaaaaagatgtataatttggaactgttaagaggcattttaaaga

SEQ ID NO: 2 - protein, 429 aa, Homo sapiens
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 3 - DNA, 1290 bp, Homo sapiens
  source 1..1290: mol_type = genomic DNA
atgcagctgaggaatcccgagctccacctgggctgtgctctggctctgcggttcctggccctcgtgtcctgggacatccctggcgctagggccctcgataacggactggcccggacccccacaatgggatggctccactgggaaaggttcatgtgcaatctggactgtcaggaggaacccgactcctgcatcagcgaaaagctcttcatggagatggccgagctgatggtgagcgagggctggaaggacgccggctacgagtatctgtgcatcgatgactgctggatggcccctcaaagggactccgaaggcaggctgcaggctgatccccaaaggtttccccacggaatccggcagctcgccaactacgtgcattccaagggcctcaagctcggcatctacgccgacgtgggcaacaaaacatgcgccggattccccggcagcttcggctactacgacatcgacgcccagacattcgctgattggggagtggacctgctgaagttcgacggctgttactgcgattccctggaaaacctggccgacggctacaaacacatgtccctcgccctgaaccggacaggcaggtccatcgtgtacagctgcgagtggcccctgtacatgtggcctttccagaagcccaactacacagagatcaggcagtactgcaaccactggaggaacttcgctgacatcgacgactcctggaagagcatcaagagcatcctggactggaccagcttcaaccaggagaggatcgtggacgtggctggacccggaggctggaacgaccccgatatgctggtgattggcaacttcggactgagctggaaccagcaggtgacccagatggccctgtgggccattatggccgctcccctgttcatgtccaacgacctgaggcacatcagcccccaggccaaggctctgctgcaggacaaggatgtgatcgccatcaaccaggaccccctgggcaagcagggctaccagctgaggcaaggagataacttcgaggtgtgggagaggcccctgtccggactggcttgggccgtggccatgatcaatcggcaggagatcggcggaccccggtcctacaccattgctgtggccagcctgggaaaaggagtcgcctgcaaccccgcctgcttcattacccagctgctccccgtgaagcggaagctgggcttctatgagtggaccagcaggctgaggtcccatatcaatcctaccggcaccgtcctcctccagctcgagaataccatgcagatgagcctcaaggatctgctgtga